(19) United States
(12) Patent Application Publication
(10) Pub. No.: US 2026/0258043 A1
(43) Pub. Date: Sep. 3, 2026

(54) PROCESS FOR THE PREPARATION OF (6S,9R)-3-OXO-3,5,6,7,8,9-HEXAHYDRO-2H-6,9-EPIMINOCYCLOHEPTA[C]PYRIDAZINE-10-CARBOXAMIDE DERIVATIVES

(71) Applicant: Pathios Therapeutics Limited, Oxford Oxfordshire, GBN (GB)

(72) Inventors: Gavin MILNE, Milton, Abingdon, Oxford Oxfordshire (GB); Tobias MOECHEL, Milton, Abingdon, Oxford Oxfordshire (GB)

(73) Assignee: Pathios Therapeutics Limited, Oxford Oxfordshire, GBN (GB)

(21) Appl. No.: 19/164,564

(22) PCT Filed: Apr. 24, 2024
(86) PCT No.: PCT/GB2024/051072

§ 371 Date: Sep. 11, 2025

(30) Foreign Application Priority Data
Apr. 25, 2023 (GB) 2306076.7

(51) Int. Cl. C07D 471/18; C07D 209/34; C07D 213/61; C07D 213/64; C07D 213/74; C07D 217/24; C07D 231/12; C07D 231/56; C07D 237/08; C07D 237/14; C07D 271/08; C07D 271/12; C07D 471/04; C07D 498/04; C07D 519/00

(52) U.S. Cl. CPC C07D 471/18; C07D 209/34; C07D 213/61; C07D 213/64; C07D 213/74; C07D 217/24; C07D 231/12; C07D 231/56; C07D 237/08; C07D 237/14; C07D 271/08; C07D 271/12; C07D 471/04; C07D 498/04; C07D 519/00

(57) ABSTRACT

The present invention relates to a process for preparing a compound of formula (I), or a pharmaceutically acceptable salt or solvate thereof, wherein: ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocy-cloalkyl, alkyl-heterocycloalkyl, alkyl-cycloalkyl, aryl, (CH₂)m-NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups; m is an integer from 0 to 3; p and q are each independently 0 to 3; Y is CR₁₀R_{10′}, wherein R₁₀ and R_{10′} are each independently selected from H, F, alkyl, and haloalkyl; Ra and Rb are each independently selected from H and alkyl; R6 is selected from H, alkyl, cycloalkyl and hydroxyalkyl; and R₁₃, R_{13′}, R₁₄, R_{14′}, R₁₅, R_{15′}, and R¹⁶ are each independently selected from H, alkyl, haloalkyl and alkoxyalkyl; said process comprising the steps of: (i) treating a compound of formula (IV), where B is defined as above, with a compound of formula (V), where R21 is phenyl optionally substituted with 1 to 5 fluorine atoms, to form a compound of formula (III); and (ii) treating said compound of formula (III) with a compound of formula (II), or a pharmaceutically acceptable salt thereof, where Y, Rₐ, R_{b} and R₆ are as described above, to form a compound of formula (I). Further aspects relate to intermediates useful in the claimed process.

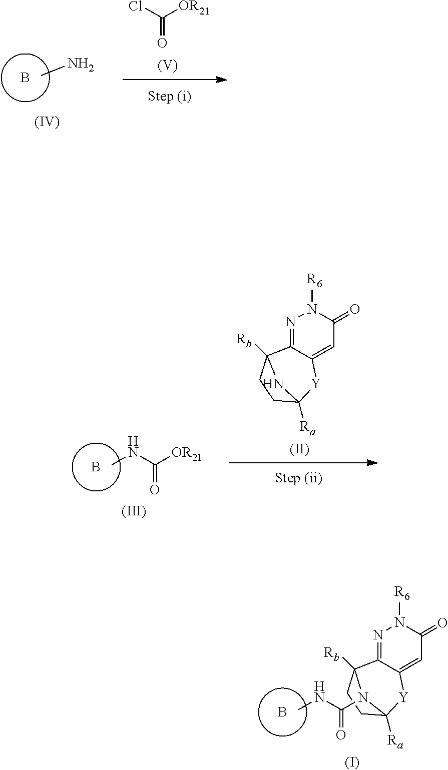

The present invention relates to a process for preparing compounds that are capable of modulating GPR65. The compounds have potential therapeutic applications in the treatment of a variety of disorders, including proliferative and immune disorders.

### BACKGROUND TO THE INVENTION

GPR65 is a Gs-coupled G protein-coupled receptor (GPCR) that is primarily expressed in immune cells and is activated by acidic extracellular pH to cause increases in cytoplasmic cyclic adenosine monophosphate (cAMP) (Wang, 2004). It has long been known that tumours typically undergo a switch in cellular metabolism from oxidative phosphorylation to aerobic glycolysis, which in turn results in an acidic extracellular microenvironment (Damaghi, 2013). Recently, it has been shown that this acidic microenvironment causes GPR65 activation in tumour-associated macrophages, resulting in an increase in cytoplasmic cAMP leading to transcription of the inducible cAMP early repressor (ICER). This, in turn, suppresses the secretion of tumour necrosis factor alpha (TNFα) to bias the macrophages toward an anti-inflammatory, tumour-permissive phenotype (Bohn, 2018). This GPR65-dependent pathway therefore appears to represent a mechanism by which tumours exploit their acidic microenvironment to evade detection by the immune system.

Autoimmune diseases are also often associated with an acidic local microenvironment (for instance, an inflamed joint). Recent studies also suggest that GPR65 acts through ICER in CD4+ T cells, to suppress IL-2 and hence bias cells toward an inflammatory Th17 phenotype, which is associated with increased pathogenicity in the context of autoimmune disease (Korn, 2009). Supporting this is the recent finding that ICER is required for Th17 differentiation (Yoshida, 2016) as well as that agonism of GPR65 leads to an increase in Th17 differentiation (Hernandez, 2018). Indeed, mutations in the GPR65 locus are associated with several autoimmune diseases, such as multiple sclerosis, ankylosing spondylitis, inflammatory bowel disease, and Crohn's disease (Gaublomme, 2015). One recent study found that mice with CD4+ T cells lacking GPR65 were protected from developing the disease autoimmune encephalomyelitis (EAE) (Gaublomme, 2015).

Thus, GPR65 appears to act through ICER to promote an anti-inflammatory and tumour-permissive phenotype in tumour associated macrophages and an inflammatory Th17 phenotype in CD4+ T cells that is associated with autoimmune disease. GPR65 signalling, therefore, represents an attractive pathway for therapeutic intervention for the treatment of both cancer and autoimmune diseases. There is therefore an ongoing need to develop new small molecule GPR65 modulators and processes for their preparation.

WO2021245427 (Pathios Therapeutics Limited) discloses a series of small molecule GPR65 modulators, and processes for their preparation. The synthetic preparation described therein involves coupling a secondary amine with a primary amine in the presence of triphosgene (bis(trichloromethyl) carbonate) to form a substituted urea derivative. An alternative route involves coupling a secondary amine with an isocyanate intermediate to form a substituted urea derivative. The isocyanate intermediate itself can be generated in situ from the corresponding amine by reacting with triphosgene in the presence of a suitable base. Triphosgene is a toxic solid that can decompose to form phosgene during the reaction.

The present invention seeks to provide further synthetic processes for preparing small molecule substituted urea derivatives capable of modulating GPR65.

### STATEMENT OF INVENTION

The present invention relates to a process for preparing a compound of formula (I), or a pharmaceutically acceptable salt or solvate thereof,

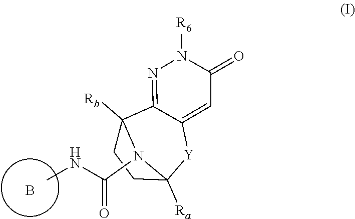

wherein:
ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
Y is CR₁₀R₁₀′, wherein R₁₀ and R₁₀′ are each independently selected from H, F, alkyl, and haloalkyl;
Rₐ and R_{b} are each independently selected from H and alkyl;
R₆ is selected from H, alkyl, cycloalkyl and hydroxyalkyl; and
R₁₃, R₁₃′, R₁₄, R₁₄′, R₁₅, R₁₅′, and R₁₆ are each independently selected from H, alkyl, haloalkyl and alkoxyalkyl;
said process comprising the steps of:
   (i) treating a compound of formula (IV), where B is defined as above, with a compound of formula (V), where R₂₁ is phenyl optionally substituted with 1 to 5 fluorine atoms, to form a compound of formula (III); and
   (ii) treating said compound of formula (III) with a compound of formula (II), or a pharmaceutically acceptable salt thereof, where Y, Rₐ, R_{b} and R₆ are as described above, to form a compound of formula (I);

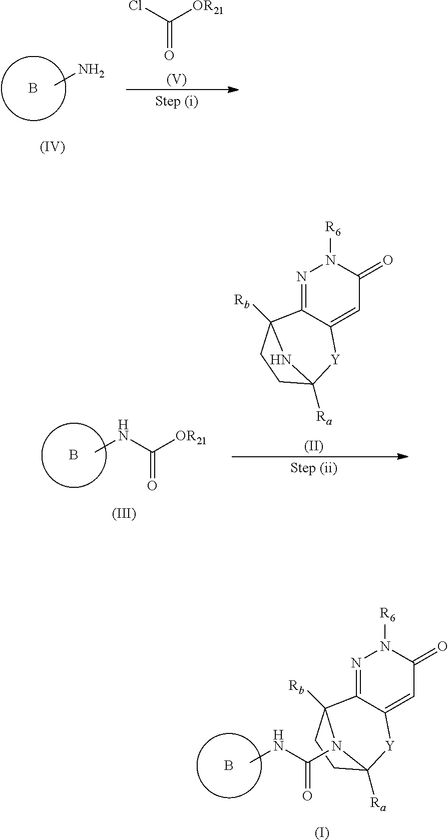

Advantageously, the presently claimed process avoids the use of triphosgene as a reagent. Triphosgene is extremely hazardous and causes severe skin burns, eye damage and can be fatal if inhaled. An alternative synthetic process which avoids the use of triphosgene is therefore particularly attractive in drug development and potential scale up operations.

Another aspect relates to carbamate intermediates useful in the process according to the invention. Thus, one aspect of the invention relates to a compound of formula (III), or a pharmaceutically acceptable salt or solvate thereof:

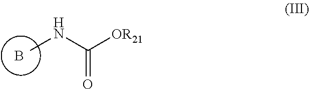

wherein:
ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
R₁₃, R₁₃′, R₁₄, R₁₄′, R₁₅, R₁₅′, and R₁₆ are each independently selected from H, alkyl, haloalkyl and alkoxyalkyl; and
R₂₁ is phenyl optionally substituted with 1 to 5 fluorine atoms.

Another aspect relates to aniline intermediates useful in the process according to the invention. Thus, a further aspect of the invention relates to a compound selected from the following:

| | | | |
|---|---|---|---|
| 1-4 | 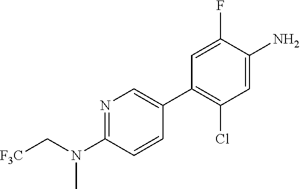 [SMILES: CN(CC(F)(F)F)c1ccc(-c2cc(F)c(N)cc2Cl)cn1] | 1-8 | 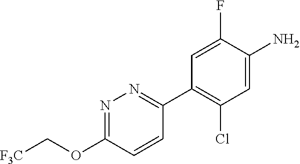 [SMILES: Nc1cc(Cl)c(-c2ccc(OCC(F)(F)F)nn2)cc1F] |
| 1-12 | 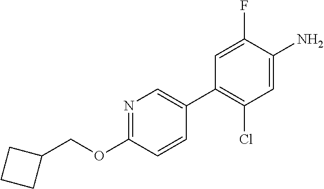 [SMILES: Nc1cc(Cl)c(-c2ccc(OCC3CCC3)nc2)cc1F] | 1-15 | 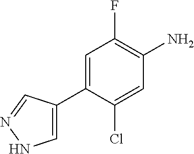 [SMILES: Nc1cc(Cl)c(-c2cn[nH]c2)cc1F] |
| 1-17 | 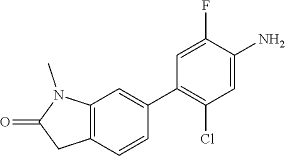 [SMILES: CN1C(=O)Cc2ccc(-c3cc(F)c(N)cc3Cl)cc21] | | |

and pharmaceutically acceptable salts and solvates thereof.

### Definitions

“Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

As used herein, the term “alkenyl” refers to both straight and branched carbon chains which have at least one carbon-carbon double bond. In some embodiments, alkenyl groups may include C₂-C₁₂ alkenyl groups. In other embodiments, alkenyl includes C₂-C₁₀, C₂-C₈, C₂-C₆ or C₂-C₄ alkenyl groups. In one embodiment of alkenyl, the number of double bonds is 1-3; in another embodiment of alkenyl, the number of double bonds is one. Other ranges of carbon-carbon double bonds and carbon numbers are also contemplated depending on the location of the alkenyl moiety on the molecule. “C₂-C₁₀-alkenyl” groups may include more than one double bond in the chain.

“Cycloalkyl” is defined herein as a monocyclic alkyl ring, preferably, C₃₋₇-cycloalkyl, more preferably C₃₋₆-cycloalkyl. Preferred examples include cyclopropyl, cyclobutyl, cyclopentyl, cyclohexyl or cycloheptyl, or a fused bicyclic ring system such as norbornane.

As used herein, the term “aryl” refers to a C₆₋₁₂ aromatic group, which may be a monocyclic or fused bicyclic group, including benzocondensed groups. Examples include phenyl and naphthyl.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferably, the haloalkyl is a C₁₋₂₀ haloalkyl, more preferably a C₁₋₁₂ haloalkyl, even more preferably a C₁₋₁₀ haloalkyl or a C₁₋₆ haloalkyl, or a C₁₋₃ haloalkyl. Preferred examples are CF₃ and CHF₂, with CF₃ being particularly preferred.

“Alkoxy” is defined herein as an oxygen atom bonded to an alkyl group as defined above, for example methoxy, ethoxy, propoxy, isopropoxy, butoxy, isobutoxy, tert-butoxy, pentoxy and hexoxy. Preferably, the alkoxy is a C₁₋₂₀ alkoxy, more preferably a C₁₋₁₂ alkoxy, even more preferably C₁₋₁₀ alkoxy or a C₁₋₆ alkoxy, or a C₁₋₃ alkoxy. A particularly preferred example is methoxy (—OCH₃).

“Alkoxy-alkyl” is defined as an alkyl group as that is substituted by one or more alkoxy groups, e.g. MeOCH₂CH₂—. “Alkoxy-alkoxy” is defined as an alkoxy group that is substituted by one or more further alkoxy groups, e.g. MeOCH₂CH₂O— (also referred to as an ether group).

“Haloalkoxy” is defined herein as an alkoxy group as described above that is substituted with one or more halogen atoms (that may be the same or different), such as fluorine, chlorine, bromine, and iodine.

“Heteroaryl” or “heteroaromatic” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is monocyclic or bicyclic. Preferably, the heterocycloalkyl group is a C₃₋₇ heterocycloalkyl, more preferably a C₃₋₆ heterocycloalkyl. Alternatively, the heterocycloalkyl group is a C₄₋₇ heterocycloalkyl, more preferably a C₄₋₆ heterocycloalkyl. Preferred heterocycloalkyl groups include, but are not limited to, piperazinyl, piperidinyl, morpholinyl, thiomorpholinyl, pyrrolidinyl, tetrahydrofuranyl and tetrahydropyranyl. Examples of heterocycloalkyl groups containing a CO group and one or more double bonds include 3-oxo-3,4-dihydro-2H-benzo[b][1,4]oxazin-6-yl, oxoisoindolinyl, oxoindolinyl, 1-oxo-1,2,3,4-tetrahydroiso-quinolin-6-yl, 1-oxo-1,2,3,4-tetrahydroisoquinolin-6-yl and the like.

“Aralkyl’ is defined herein as an alkyl group as defined above substituted by one or more aryl groups as defined above.

Preferably alkyl is C₁-C₆ alkyl, haloalkyl is C₁-C₆ haloalkyl, alkoxy is C₁-C₆ alkoxy and haloalkoxy is C₁-C₆ haloalkoxy.

### Structural Representation of the Compounds

The compounds described herein comprise a structure wherein an optionally substituted 6-membered nitrogen-containing ring is fused to a bicyclic nitrogen-containing moiety to form a tricyclic structure. The resulting tricyclic structure can exist in two different configurations as depicted below:

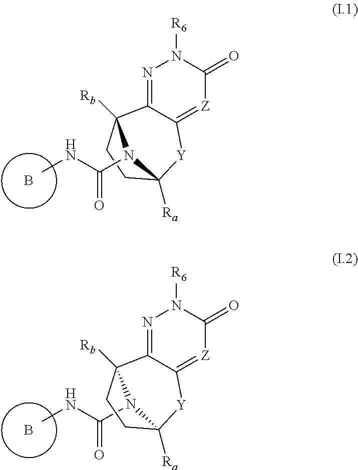

For the avoidance of doubt, the invention encompasses the compounds in either of the above configurations, as well as mixtures thereof, including racemic mixtures.

Alternatively, the structure can, for example, be represented, as follows (where Rₐ and R_{b} groups are omitted for clarity):

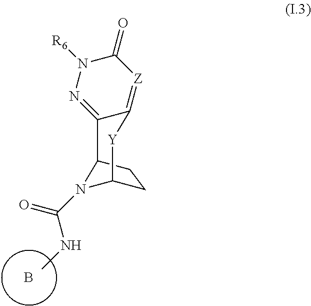

For the avoidance of doubt, the invention encompasses the compounds in the above configuration, as well the corresponding enantiomers thereof, and mixtures thereof, including racemic mixtures. As used throughout, and for ease of representation, specific examples of compounds according to the invention depicted in the above configuration (1.3) refer to mixtures of both enantiomers (in particular, the racemate), whereas the respective enantiomers—where these have been synthesised or separated—are depicted as either configuration (1.1) or configuration (1.2) with wedged bonds or dashed bonds respectively.

The compounds described herein contain an optionally substituted 6-membered ring, which is fused to the bicyclic nitrogen-containing moiety to form a tricyclic structure. The 6-membered ring contains a C═O group and can exist in more than one tautomeric form. For example, compounds of formula (I) where R₆ is H can exist in the following tautomeric forms:

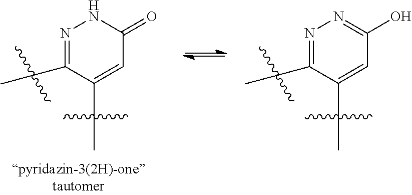 [SMILES: *c1cc(=O)[nH]nc1* ; *c1cc(O)nnc1*]

The pyridazin-3 (2H)-one tautomer is believed to be the predominant solid state form. In solution, the energy difference between the two tautomeric forms is understood to be very small and is dependent on the polarity of the solvent. The invention encompasses all tautomeric forms of the compounds described herein.

### DETAILED DESCRIPTION

Preferred embodiments of the invention are set out below.

In one particularly preferred embodiment, the compound prepared by the process of the invention is of formula (I.1):

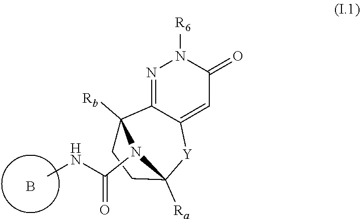

wherein B, Y, Rₐ, R_{b} and R₆ are as defined above.

In one preferred embodiment, the compound is in enantiomerically pure form. In one preferred embodiment, the compound is in the form of a mixture that is enantiomerically enriched with a compound of formula (I.1).

In another embodiment, the compound is of formula (I.2):

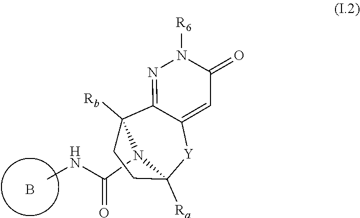

wherein B, Y, Rₐ, R_{b} and R₆ are as defined above.

Enantiomeric forms (I.1) and (I.2) apply equally to all of the various subformulae described herein.

In one preferred embodiment, the compound is in enantiomerically pure form. In one preferred embodiment, the compound is in the form of a mixture that is enantiomerically enriched with a compound of formula (I.2).

In one preferred embodiment, the compound is in the form of a mixture comprising a compound of formula (I.1) and its corresponding enantiomer of formula (I.2). In one preferred embodiment, the mixture is a racemic mixture, i.e. a 50:50 mixture of a compound of formula (I.1) and its corresponding enantiomer of formula (I.2).

Racemic mixtures can be used to prepare enantiomerically pure compounds of formula (I.1) or (I.2) by separating the compounds of formula (I.1) or (I.2) by standard methods, for example by chemical resolution using optically active acid or by the use of column chromatography or reverse-phase column chromatography using a substantially optically active (or “chiral”) stationary phase as known to those skilled in the art. Racemic mixtures can also be used to prepare enantiomerically enriched mixtures of compounds of formula (I.1) or (I.2). Mixtures enriched with either a compound of formula (I.1) or (I.2) can also be obtained from the appropriate enantiomerically enriched precursors.

In one preferred embodiment of the invention, the compound is in the form of a mixture comprising enantiomers wherein the weight:weight ratio is at least approximately 2:1 or greater, preferably at least approximately 5:1 or greater, most preferably at least approximately 10:1 or greater in favour of the enantiomer that displays significant in vitro and/or in vivo activity (the eutomer).

In one particularly preferred embodiment, the compound is in the form of a mixture comprising a compound of formula (I.1) and its corresponding enantiomer of formula (I.2), wherein the weight:weight ratio of said compound of formula (I.1) to said compound of formula (I.2) is greater than 1.05:1, more preferably, greater than 2:1, even more preferably greater than 5:1, even more preferably greater than 10:1.

In one particularly preferred embodiment, the compound is in the form of a mixture comprising a compound of formula (I.1) and its corresponding enantiomer of formula (I.2), which is substantially enriched with said compound of formula (I.1).

In one embodiment, the compound is in the form of a mixture comprising a compound of formula (I.1) and its corresponding enantiomer of formula (I.2), wherein the weight:weight ratio of said compound of formula (I.2) to said compound of formula (I.1) is greater than 1.05:1, more preferably, greater than 2:1, even more preferably greater than 5:1, even more preferably greater than 10:1.

In one embodiment, the compound is in the form of a mixture comprising a compound of formula (I.1) and its corresponding enantiomer of formula (I.2), which is substantially enriched with said compound of formula (I.2).

In one preferred embodiment, Rₐ and R_{b} are both H.

In one preferred embodiment, Y is CH₂.

In one preferred embodiment, R₆ is selected from H, methyl and hydroxymethyl, and is more preferably H.

In one preferred embodiment, B is a phenyl group optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

In one preferred embodiment, Ring B is:

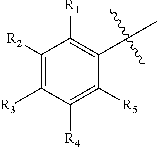

wherein:
R₁, R₄, and R₅ are each independently selected from H, CN, alkyl, alkoxy, haloalkyl, OH, and halo;
R₂ and R₃ are each independently selected from H, OH, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

In one preferred embodiment, R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl and alkoxy.

In one preferred embodiment, R₂ and R₃ are each independently selected from F, Cl, Br, I, CN, C₁-C₆ haloalkyl, optionally substituted heteroaryl, C₁-C₆ haloalkoxy and CO₂-alkyl, more preferably, CI, Br, and CF₃, even more preferably optionally substituted heteroaryl, CI and CF₃.

In one preferred embodiment, R₃ is an aryl or heteroaryl group, preferably a pyridinyl group, each of which is optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

In one preferred embodiment, R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzooxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3(4H)-onyl, more preferably a pyridinyl group; each of which is optionally further substituted by one or more groups independently selected from halo, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, and heteroaryl, and O—(CH₂)ₚ-cycloalkyl, where in the latter group, said cycloalkyl group is optionally further substituted by one or more halo, haloalkyl, alkyl or alkoxy groups.

In one preferred embodiment, R₃ is selected from:

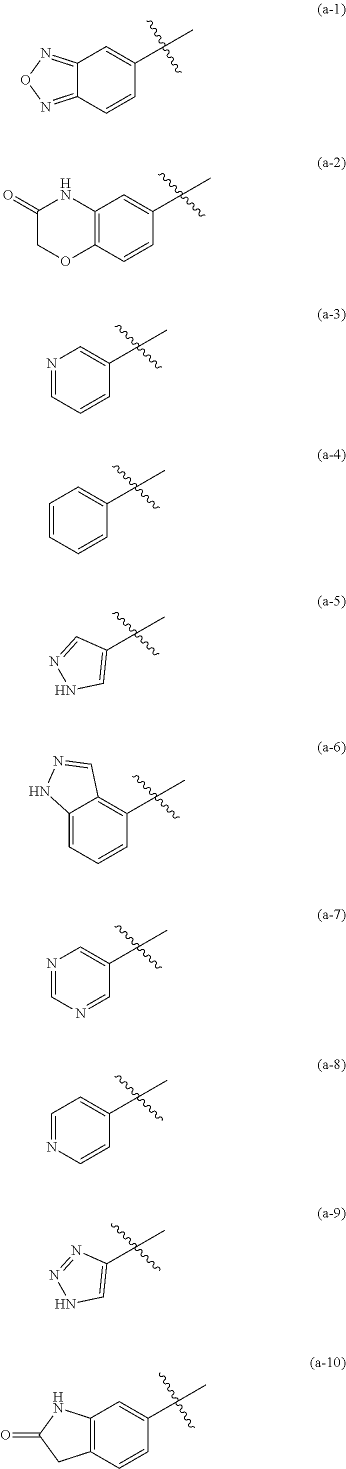

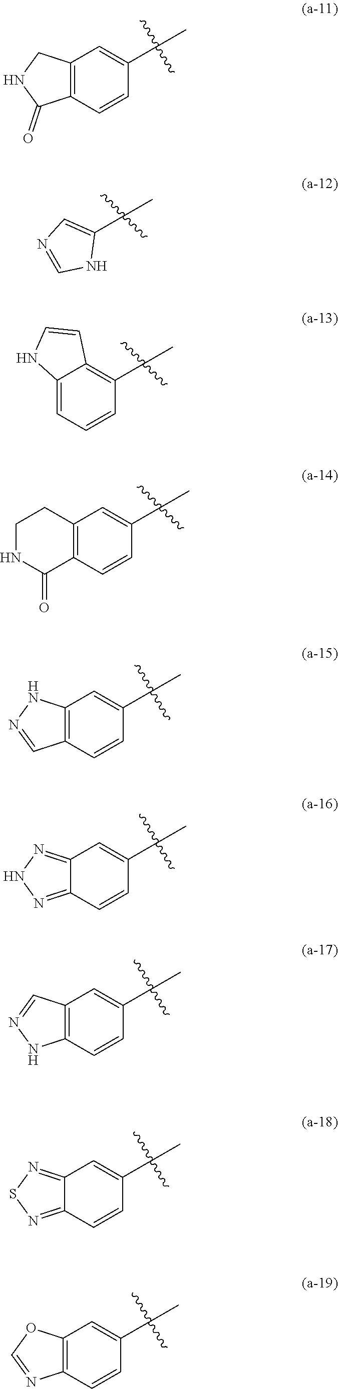

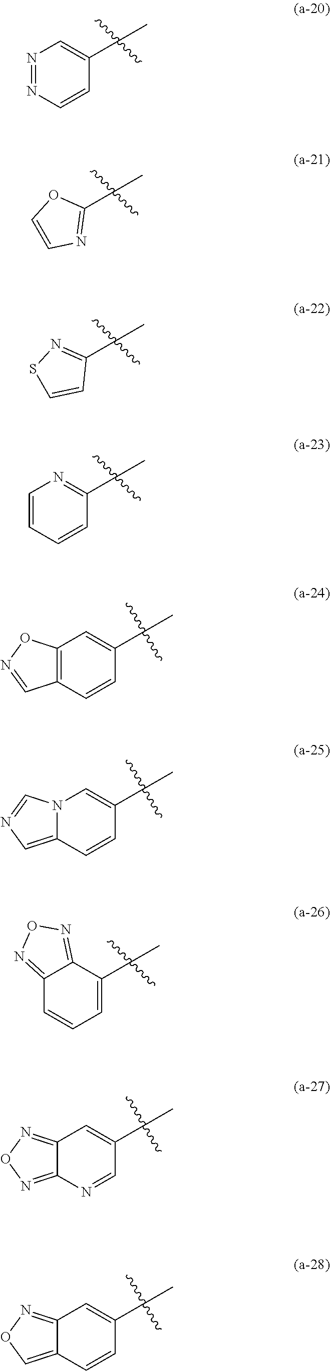

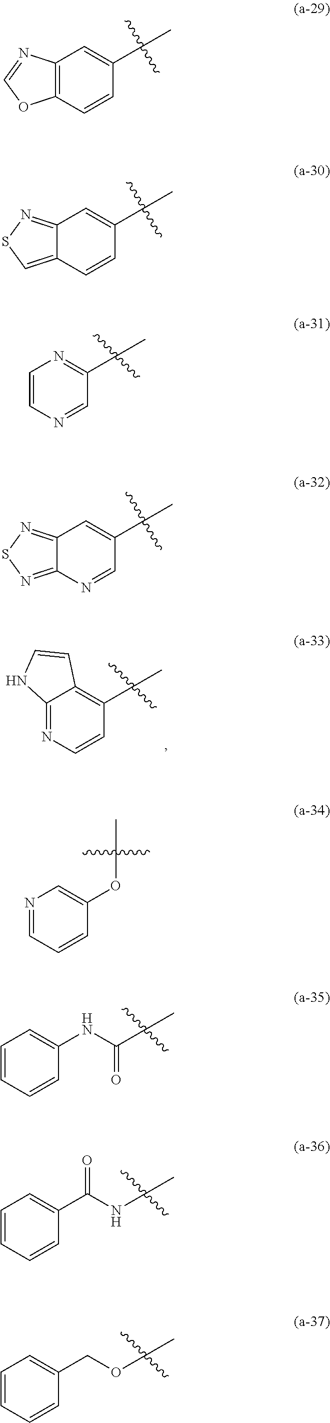

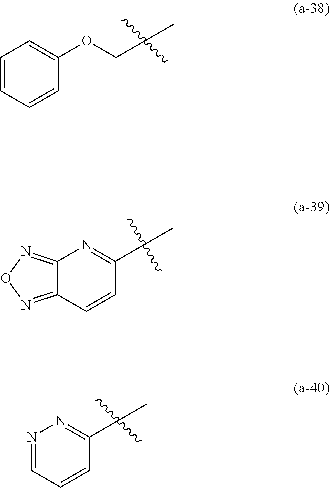 [SMILES: *COc1ccccc1 ; *c1ccc2nonc2n1 ; *c1cccnn1]

each of which is optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, —(CH₂)_{q}—O-heteroaryl, and O—(CH₂)ₚ-cycloalkyl, where in the latter group, said cycloalkyl group is optionally further substituted by one or more halo, haloalkyl, alkyl or alkoxy groups. Preferably, R₃ is selected from groups (a-1), (a-3), (a-5), (a-6), (a-10), (a-14), (a-17), (a-20), (a-25), (a-26), (a-27), (a-39) and (a-40) each of which is optionally substituted.

In one preferred embodiment, R₃ is:

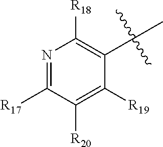

wherein:
R₁₇ is selected from H, alkyl, CN, haloalkyl, NHCO-alkyl, NR₁₃R₁₃′, alkoxy, SO₂-alkyl, halo, O—(CH₂)_{q}-heterocycloalkyl, alkoxy-alkoxy, alkoxy-alkyl, haloalkoxy, alkylamino-alkoxy, dialkylamino-alkoxy, and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆.

In one preferred embodiment, R₁₃, R₁₃′, R₁₄, R₁₄′, R₁₅, R₁₅′, and R₁₆ are each independently selected from H, alkyl and alkoxyalkyl.

In one preferred embodiment, R₁₃, R₁₃′, R₁₄, R₁₄′, R₁₅, R₁₅′, and R₁₆ are each independently selected from H, alkyl and haloalkyl.

In one preferred embodiment, R₁₃ and R₁₃′ are each independently selected from H, alkyl and haloalkyl.

In one preferred embodiment, p is 0 or 1.

In one preferred embodiment, q is 0 or 1.

In one preferred embodiment, m is 0 or 1.

In one preferred embodiment:
R₁₇ is selected from H, OMe, OEt, OⁱPr, F, SO₂Me, halo, O-cyclobutyl, O-oxetanyl, O—CH₂CF₃, CH₂CF₃, O—CH₂-cyclobutyl, O-cyclopentyl, NHCH₂CF₃, N(Me)CH₂CF₃ and O—CH₂-(difluorocyclopropyl), and O—CH₂-cyclopropyl;
R₁₈ is selected from H and F;
R₁₉ is selected from H and MeOCH₂—;
R₂₀ is selected from H, F and CO₂Me;
R₁ is F;
R₂ is H;
R₄ is CI or CF₃; and
R₅ is H.

In one preferred embodiment:
R₁₇ is selected from H, OMe, OEt, OⁱPr, F, SO₂Me, halo, O-cyclobutyl, O-oxetanyl, and O—CH₂CF₃;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

In one preferred embodiment, R₂ and R₅ are both H.

In one preferred embodiment, R₁ is selected from H, F, Me, MeO, CI, OH and CN, and is preferably H or F.

In one preferred embodiment, R₄ is selected from Cl, Br, and CF₃, more preferably Cl.

In one preferred embodiment, R₁ is F, R₂ is H, R₄ is CI and R₅ is H.

In one preferred embodiment, the compound of formula (V) is selected from phenyl chloroformate and pentafluorophenyl choroformate.

In one highly preferred embodiment, the compound of formula (V) is phenyl chloroformate:

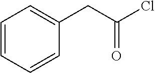

In one preferred embodiment, step (i) is carried out in the absence of base.

In one preferred embodiment, step (i) is carried out in a solvent. Preferred solvents include organic solvents such as tetrahydrofuran, dichloromethane, DMSO, and 2-methyltetrahydrofuran, and mixtures thereof. The skilled person will appreciate that other suitable solvents and solvent mixtures may also be used.

In one preferred embodiment, the ratio of compound (V) to compound (IV) in step (i) is about 3:1 to about 1:1. More preferably, about 2:1 to about 1:1, more preferably about 1.5:1 to about 1:1, even more preferably about 1.2:1 to about 1:1. In one particularly preferred embodiment, the ratio of compound (V) to compound (IV) in step (i) is about 1.2:1 to about 1:1, more preferably about 1.1:1 to about 1:1.

In one preferred embodiment, step (i) is carried out at a temperature of from about 0° C. to about 45° C., preferably from about 10° C. to about 40° C., more preferably from about 20 to about 40° C., more preferably from about 30 to about 40° C., more preferably from about 25 to about 35° C.

In one preferred embodiment, step (ii) is carried out in the presence of a base. Preferably, the base is an amine base. More preferably the base is selected from N,N-diisopropylethylamine (DIEA), triethylamine, tri-“propylamine, triisopropylamine, N-methyl morpholine, N-methylpiperidine and tri-” butylamine. In one particularly preferred embodiment, the base is triethylamine.

In one preferred embodiment, step (ii) is carried out in the absence of a base.

In one preferred embodiment, step (ii) is carried out in a solvent. Preferred solvents include organic solvents such as tetrahydrofuran, dichoromethane, dimethylsulfoxide (DMSO), and 2-methyltetrahydrofuran, and mixtures thereof. The skilled person will appreciate that other suitable solvents may also be used. In one preferred embodiment, the solvent is 2-methyltetrahydrofuran.

In one preferred embodiment, step (ii) is carried out at a temperature of from about 35° C. to about 65° C., more preferably about 40° C. to about 60° C., more preferably about 40° C. to about 50° C.

In one preferred embodiment, steps (i) and (ii) are carried out sequentially without isolating the compound of formula (III) from the reaction mixture obtained in step (i). Preferably, step (ii) is carried out on the crude reaction mixture obtained from step (i).

In one preferred embodiment, steps (i) and (ii) are carried out sequentially without isolating the compound of formula (III) from the reaction mixture obtained in step (i) and without purifying the compound of formula (III). Preferably, step (ii) is carried out on the crude reaction mixture obtained from step (i) without further isolation and purification of the compound of formula (III).

In an alternative preferred embodiment, the compound of formula (III) is isolated from the reaction mixture obtained in step (i) prior to carrying out step (ii). Preferably, the compound of formula (III) is isolated and purified from the reaction mixture obtained in step (i) prior to carrying out step (ii).

In one preferred embodiment, the compound of formula (II) is in the form of a pharmaceutically acceptable salt, more preferably, a hydrochloride salt.

In one preferred embodiment, the ratio of compound (II), or pharmaceutically acceptable salt thereof, to compound (III) in step (ii) is about 1:1.

In one preferred embodiment, the compound of formula (I) is selected from the following:

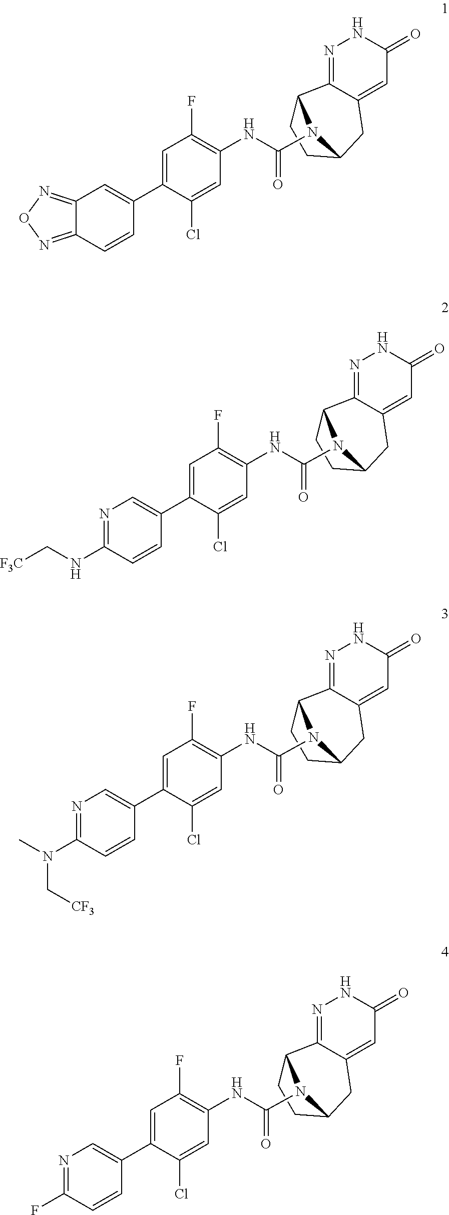 [SMILES: O=C(Nc1cc(Cl)c(-c2ccc3nonc3c2)cc1F)N1[C@H]2CC[C@@H]1c1n[nH]c(=O)cc1C2 ; O=C(Nc1cc(Cl)c(-c2ccc(NCC(F)(F)F)nc2)cc1F)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc(F)nc2)cc1F)N1[C@H]2CC[C@@H]1c1n[nH]c(=O)cc1C2]

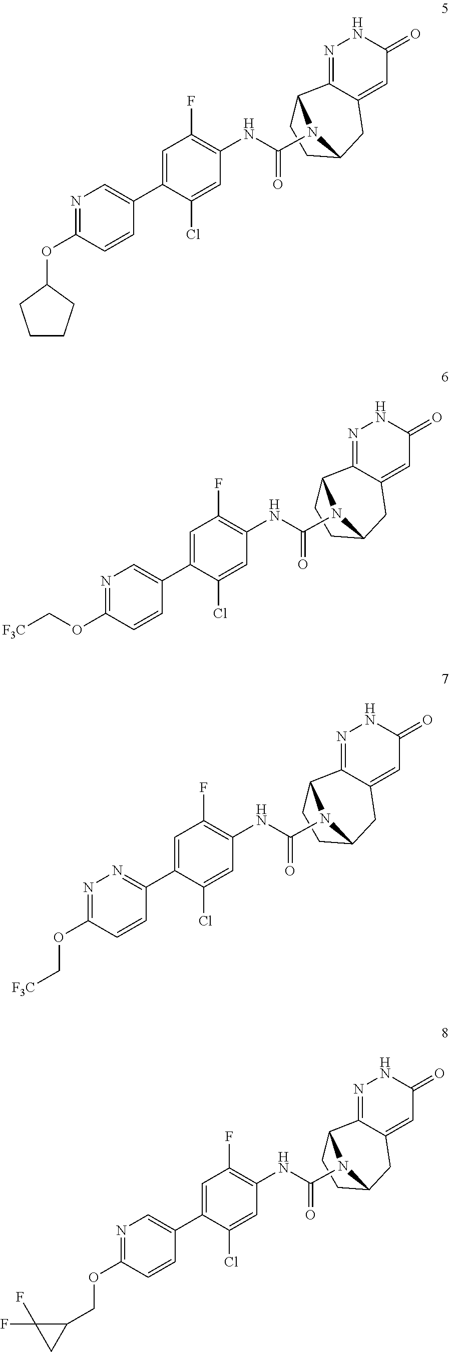 [SMILES: O=C(Nc1cc(Cl)c(-c2ccc(OC3CCCC3)nc2)cc1F)N1[C@H]2CC[C@@H]1c1n[nH]c(=O)cc1C2 ; O=C(Nc1cc(Cl)c(-c2ccc(OCC(F)(F)F)nc2)cc1F)N1[C@H]2CC[C@@H]1c1n[nH]c(=O)cc1C2 ; O=C(Nc1cc(Cl)c(-c2ccc(OCC(F)(F)F)nn2)cc1F)N1[C@H]2CC[C@@H]1c1n[nH]c(=O)cc1C2 ; O=C(Nc1cc(Cl)c(-c2ccc(OCC3CC3(F)F)nc2)cc1F)N1[C@H]2CC[C@@H]1c1n[nH]c(=O)cc1C2]

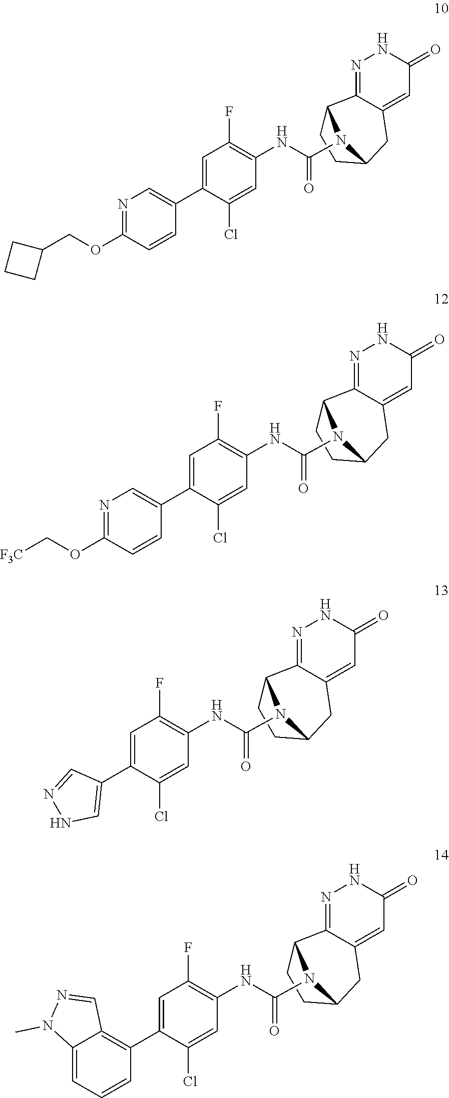 [SMILES: O=C(Nc1cc(Cl)c(-c2ccc(OCC3CCC3)nc2)cc1F)N1[C@H]2CC[C@@H]1c1n[nH]c(=O)cc1C2 ; O=C(Nc1cc(Cl)c(-c2ccc(OCC(F)(F)F)nc2)cc1F)N1[C@H]2CC[C@@H]1c1n[nH]c(=O)cc1C2 ; O=C(Nc1cc(Cl)c(-c2cn[nH]c2)cc1F)N1[C@H]2CC[C@@H]1c1n[nH]c(=O)cc1C2 ; Cn1ncc2c(-c3cc(F)c(NC(=O)N4[C@H]5CC[C@@H]4c4n[nH]c(=O)cc4C5)cc3Cl)cccc21]

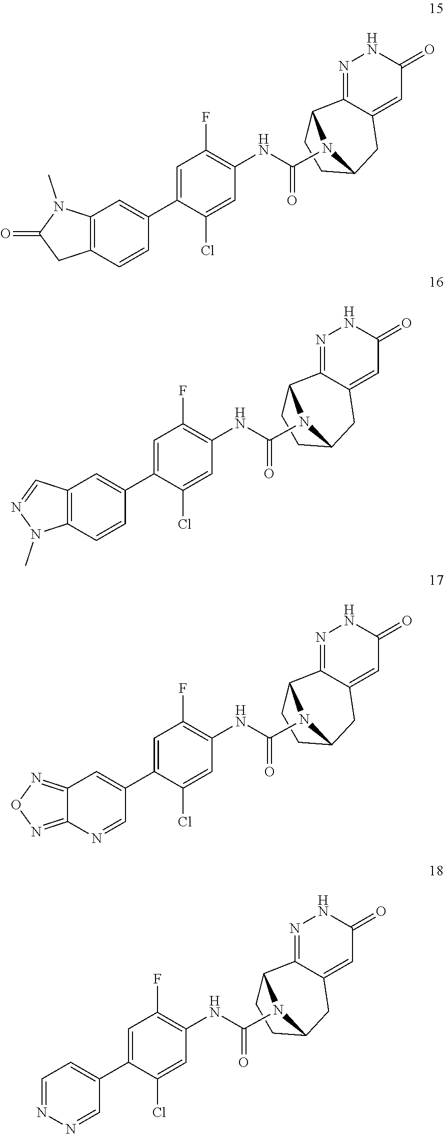 [SMILES: CN1C(=O)Cc2ccc(-c3cc(F)c(NC(=O)N4[C@H]5CC[C@@H]4c4n[nH]c(=O)cc4C5)cc3Cl)cc21 ; Cn1ncc2cc(-c3cc(F)c(NC(=O)N4[C@H]5CC[C@@H]4c4n[nH]c(=O)cc4C5)cc3Cl)ccc21 ; O=C(Nc1cc(Cl)c(-c2cnc3nonc3c2)cc1F)N1[C@H]2CC[C@@H]1c1n[nH]c(=O)cc1C2 ; O=C(Nc1cc(Cl)c(-c2ccnnc2)cc1F)N1[C@H]2CC[C@@H]1c1n[nH]c(=O)cc1C2]

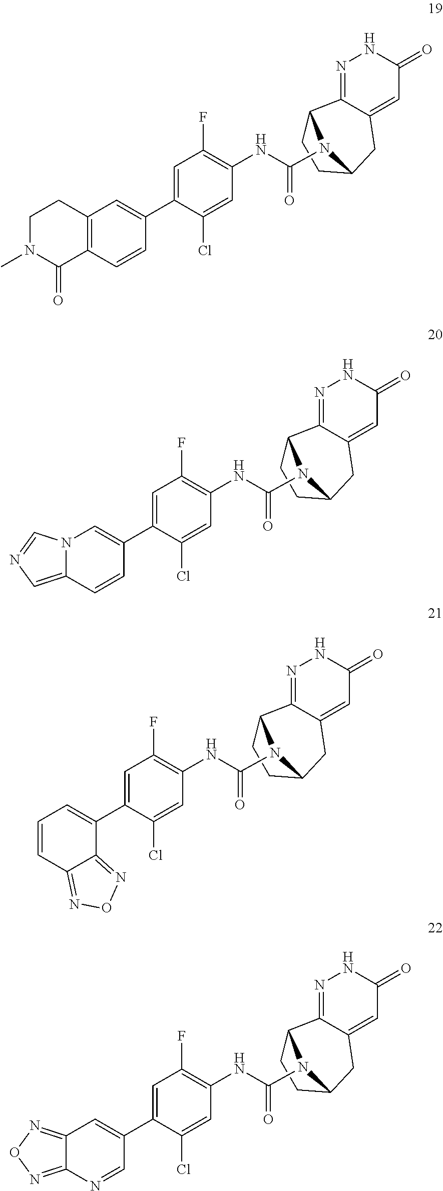 [SMILES: CN1CCc2cc(-c3cc(F)c(NC(=O)N4[C@H]5CC[C@@H]4c4n[nH]c(=O)cc4C5)cc3Cl)ccc2C1=O ; O=C(Nc1cc(Cl)c(-c2ccc3cncn3c2)cc1F)N1[C@H]2CC[C@@H]1c1n[nH]c(=O)cc1C2 ; O=C(Nc1cc(Cl)c(-c2cccc3nonc23)cc1F)N1[C@H]2CC[C@@H]1c1n[nH]c(=O)cc1C2 ; O=C(Nc1cc(Cl)c(-c2cnc3nonc3c2)cc1F)N1[C@H]2CC[C@@H]1c1n[nH]c(=O)cc1C2]

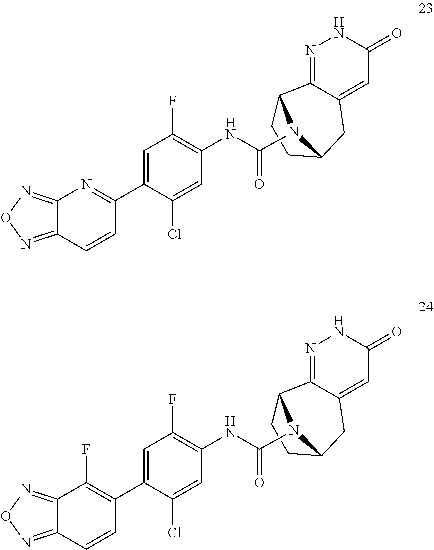 [SMILES: O=C(Nc1cc(Cl)c(-c2ccc3nonc3n2)cc1F)N1[C@H]2CC[C@@H]1c1n[nH]c(=O)cc1C2 ; O=C(Nc1cc(Cl)c(-c2ccc3nonc3c2F)cc1F)N1[C@H]2CC[C@@H]1c1n[nH]c(=O)cc1C2]

and enantiomers thereof, and mixtures of enantiomers thereof, including racemic mixtures, and pharmaceutically acceptable salts and solvates thereof.

Further aspects of the invention relate to intermediates useful in the process described herein.

Thus, one aspect of the invention relates to a compound of formula (III), or a pharmaceutically acceptable salt or solvate thereof:

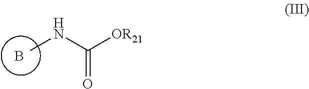

wherein:
ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
R₁₃, R₁₃′, R₁₄, R₁₄′, R₁₅, R₁₅′, and R₁₆ are each independently selected from H, alkyl, haloalkyl and alkoxyalkyl; and
R₂₁ is phenyl optionally substituted with 1 to 5 fluorine atoms.

In one preferred embodiment, Ring B is:

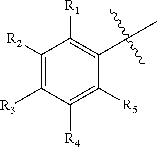

wherein:
R₁, R₄, and R₅ are each independently selected from H, CN, alkyl, alkoxy, haloalkyl, OH, and halo;
R₂ and R₃ are each independently selected from H, OH, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

In one preferred embodiment, R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl, alkoxy and haloalkoxyl.

In one preferred embodiment, R₃ is an aryl or heteroaryl group, each of which is optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

In one preferred embodiment, R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzooxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3 (4H)-onyl, more preferably a pyridinyl group; each of which is optionally further substituted by one or more groups independently selected from halo, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, heteroaryl, and O—(CH₂)ₚ-cycloalkyl, where in the latter group, said cycloalkyl group is optionally further substituted by one or more halo, haloalkyl, alkyl or alkoxy groups.

In one preferred embodiment, R₃ is selected from:

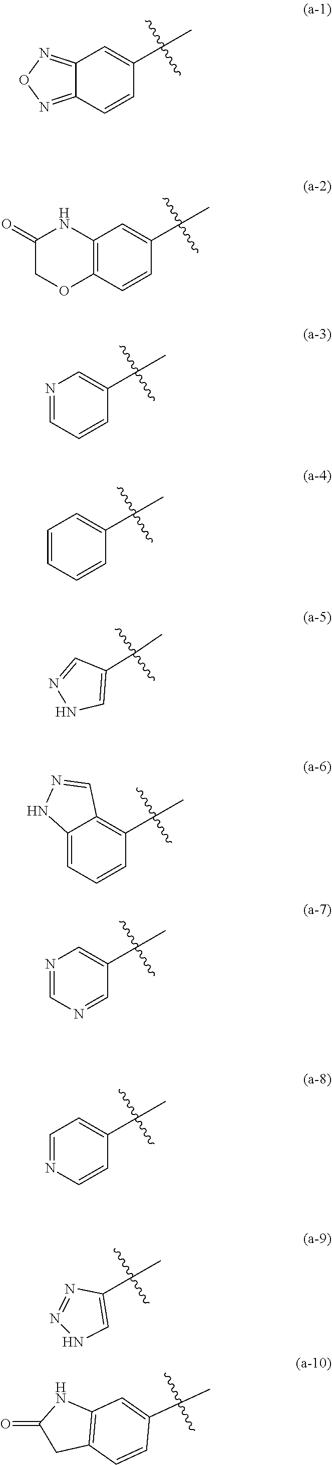

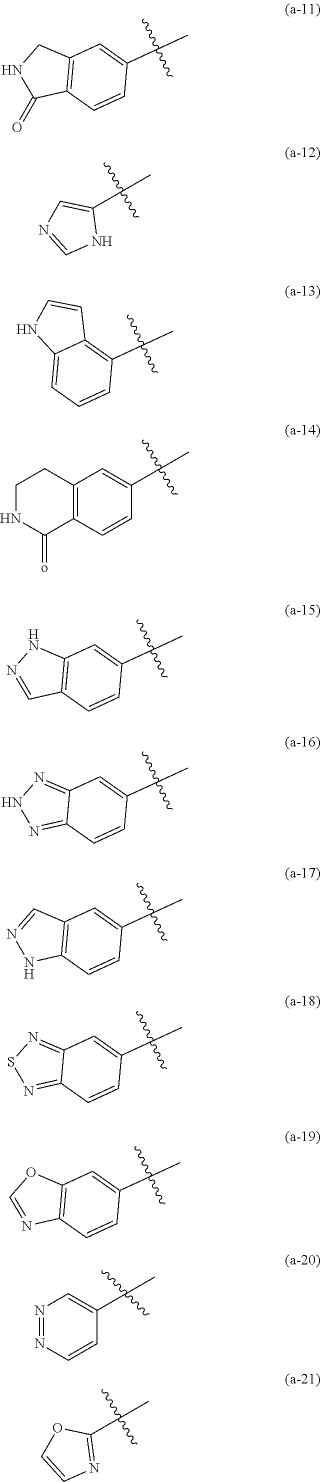

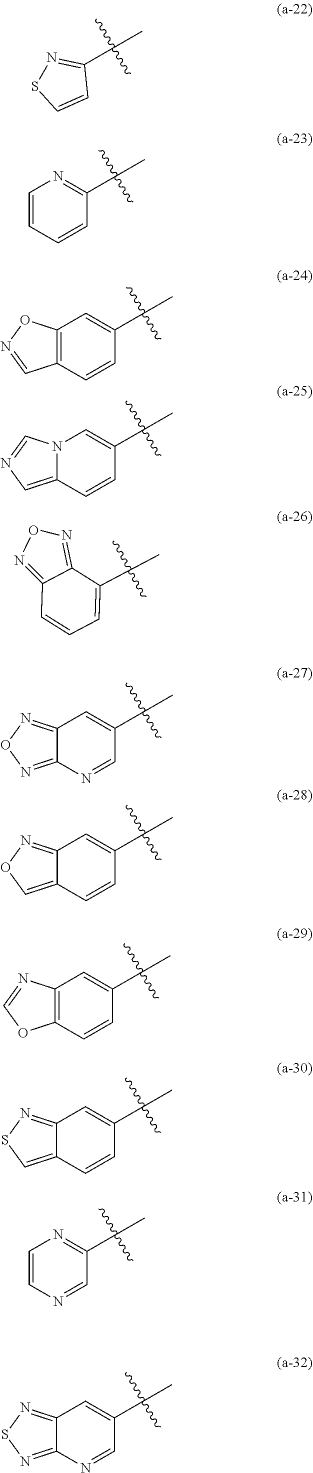

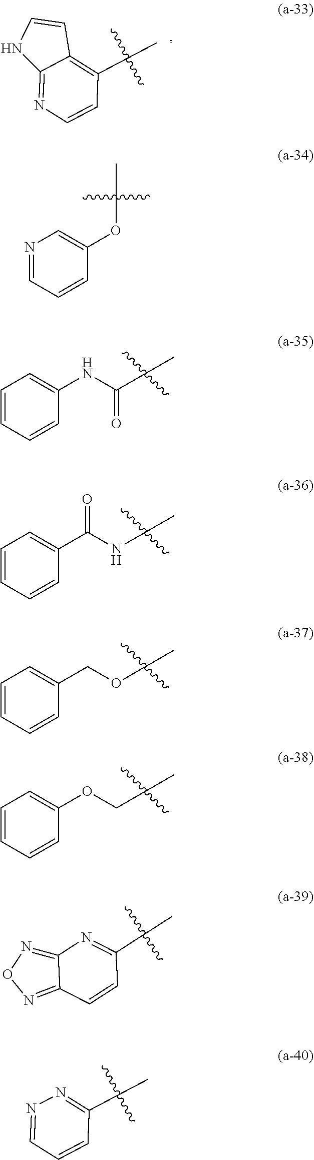

each of which is optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, —(CH₂)_{q}—O-heteroaryl, and O—(CH₂)ₚ-cycloalkyl, where in the latter group, said cycloalkyl group is optionally further substituted by one or more halo, haloalkyl, alkyl or alkoxy groups. Preferably, R₃ is selected from groups (a-1), (a-3), (a-5), (a-6), (a-10), (a-14), (a-17), (a-20), (a-25), (a-26), (a-27), (a-39) and (a-40) each of which is optionally substituted.

In one preferred embodiment, R₃ is:

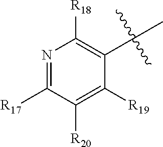

wherein:
R₁₇ is selected from H, alkyl, CN, haloalkyl, NHCO-alkyl, NR₁₃R₁₃′, alkoxy, SO₂-alkyl, halo, O—(CH₂)_{q}-heterocycloalkyl, alkoxy-alkoxy, alkoxy-alkyl, haloalkoxy, alkylamino-alkoxy, dialkylamino-alkoxy, and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆.

In one preferred embodiment:
R₁₇ is selected from H, OMe, OEt, OⁱPr, F, SO₂Me, halo, O-cyclobutyl, O-oxetanyl, O—CH₂CF₃, CH₂CF₃, O—CH₂-cyclobutyl, O-cyclopentyl, NHCH₂CF₃, N(Me)CH₂CF₃, O—CH₂-(difluorocyclopropyl), and O—CH₂-cyclopropyl;
R₁₈ is selected from H and F;
R₁₉ is selected from H and MeOCH₂—;
R₂₀ is selected from H, F and CO₂Me;
R₁ is F;
R₂ is H;
R₄ is CI or CF₃; and
R₅ is H.

In one preferred embodiment, R₂ and R₅ are both H.

In one preferred embodiment, R₁ is selected from H, F, Me, MeO, Cl, OH and CN, and is preferably H or F.

In one preferred embodiment, R₄ is selected from Cl, Br, and CF₃, more preferably Cl.

In one preferred embodiment, R₁ is F, R₂ is H, R₄ is Cl and R₅ is H.

In one preferred embodiment, R₂₁ is phenyl or pentafluorophenyl, more preferably, phenyl.

In one preferred embodiment, the compound is selected from the following:

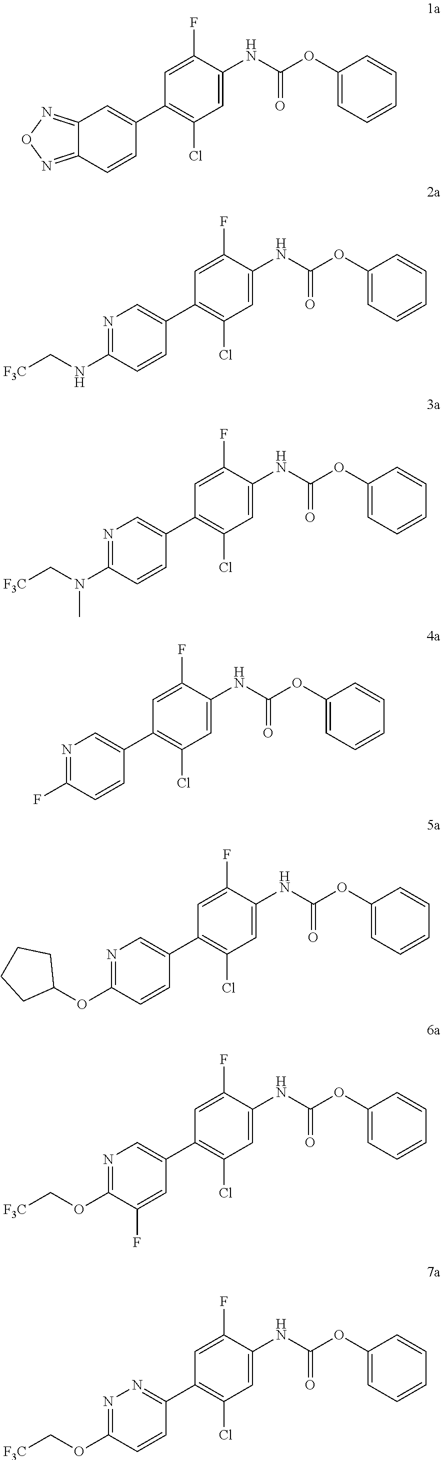

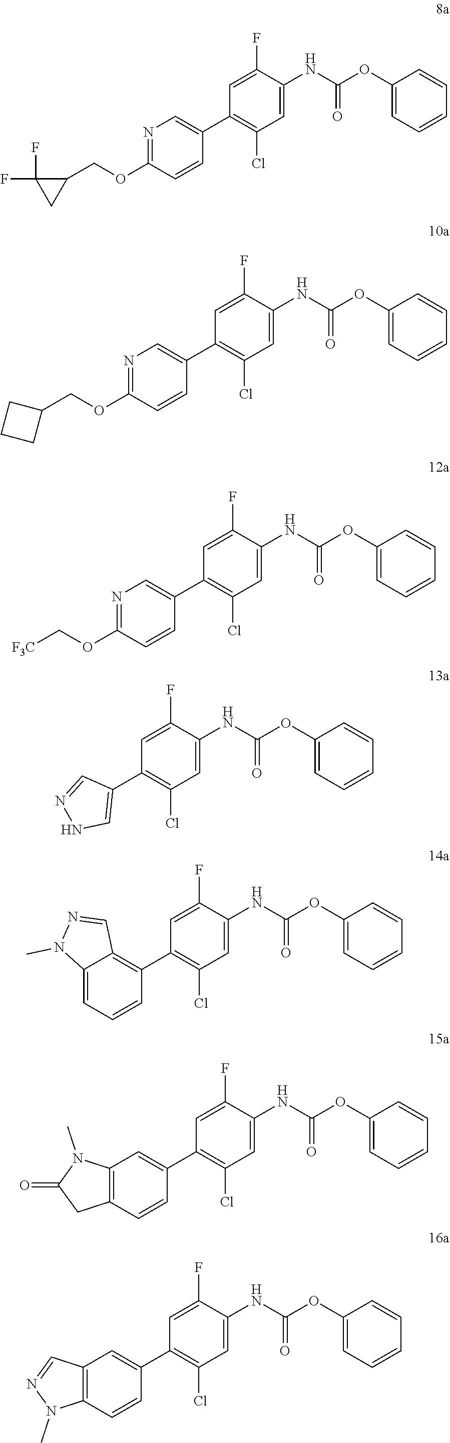

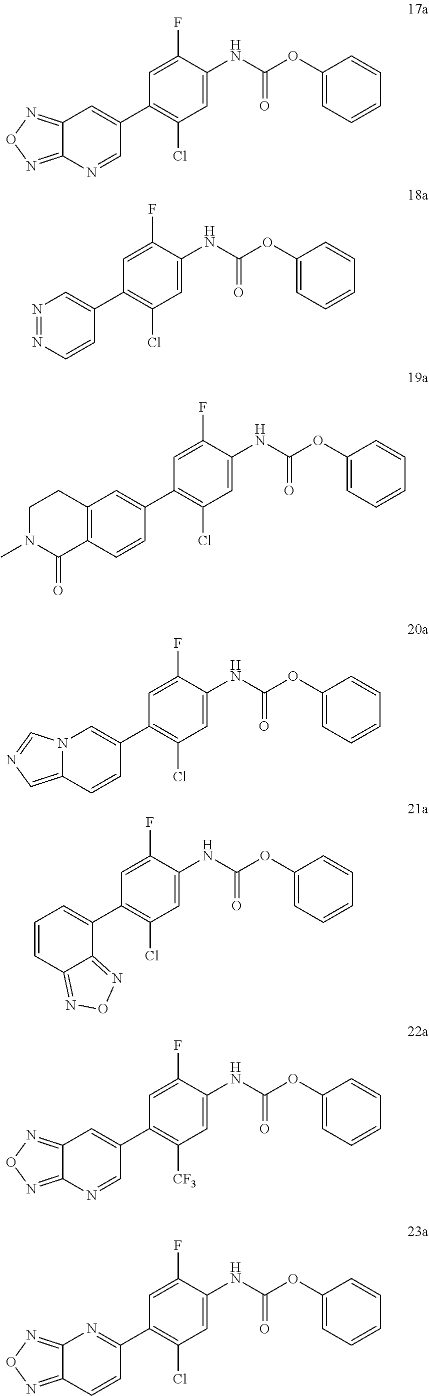

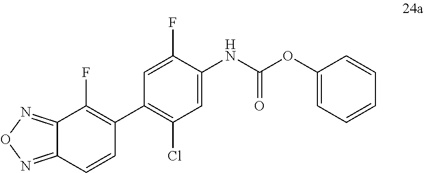 [SMILES: O=C(Nc1cc(Cl)c(-c2ccc3nonc3c2F)cc1F)Oc1ccccc1]

and enantiomers thereof, and mixtures of enantiomers thereof, including racemic mixtures, and pharmaceutically acceptable salts and solvates thereof.

Another aspect of the invention relates to the use of compound of formula (III) as defined above in the preparation of a compound of formula (I) as defined above.

Another aspect relates to aniline intermediates useful in the process according to the invention. Thus, a further aspect of the invention relates to a compound selected from the following:

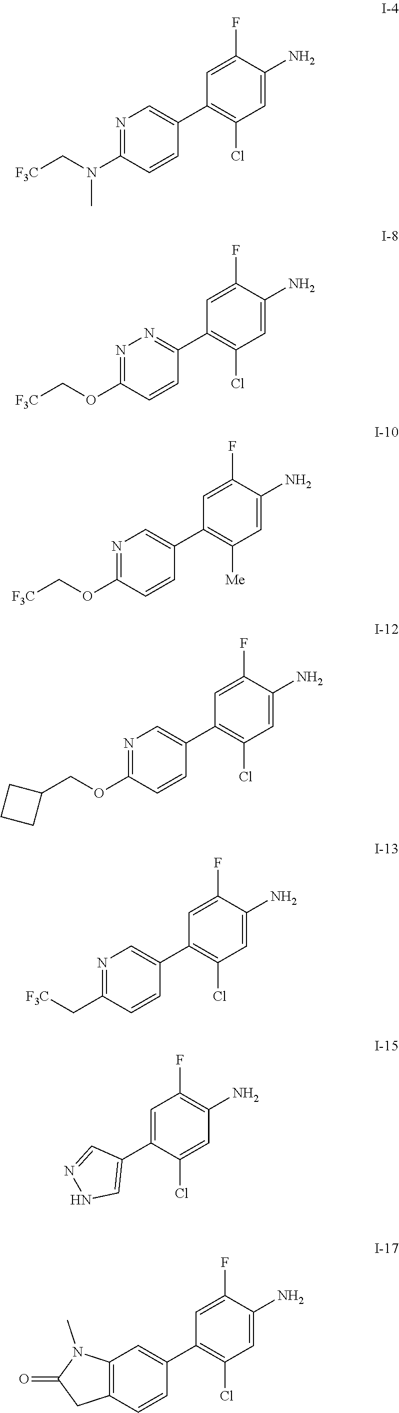

### Salts

In all aspects of the present invention previously discussed, the invention includes, where appropriate all enantiomers, diastereoisomers and tautomers of the compounds of described herein.

As used herein, pharmaceutically acceptable salts include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

### Enantiomers/Tautomers

In all aspects of the present invention previously discussed, the invention includes, where appropriate all enantiomers, diastereoisomers and tautomers of the compounds of described herein. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formulae (I) and (II) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds described herein may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers. The invention also covers rotamers of the compounds.

### Solvates

The present invention also includes solvate forms of the compounds described herein. The terms used in the claims encompass these forms. Preferably, the solvate is a hydrate.

### Polymorphs

The invention further relates to the compounds described herein in their various crystalline forms, polymorphic forms and (an) hydrous forms. It is well established within the pharmaceutical industry that chemical compounds may be isolated in any of such forms by slightly varying the method of purification and or isolation form the solvents used in the synthetic preparation of such compounds.

The invention is further described with reference to the following non-limiting examples.

### General Schemes

### Abbreviations

A list of some common abbreviations is shown below—where other abbreviations are used which are not listed, these will be understood by the person skilled in the art.

br.: broad; d: doublet; DCM: dichloromethane; DMSO: dimethylsulfoxide; (ES+): electrospray ionization positive mode; Et₃N: triethylamine; EtOAc: ethyl acetate; EtOH: ethanol; h: hours; hept: heptet; HPLC: high performance liquid chromatography; HCl: hydrochloric acid; Hz: hertz; J: coupling constant; I: litre; M: molar; m: multiplet; [M+H]+: protonated molecular ion; MeCN: acetonitrile; MeOH: methanol; MHz: megahertz; 2-MeTHF: 2-methyl tetrahydrofuran; min: minutes; mL: millilitres; MS: mass spectrometry; MTBE: methyl tert-butyl ether; m/z: mass-to-charge ratio; NMR: nuclear magnetic resonance; p: pentet; PDA: photodiode array; Pd (dppf) Cl₂: [1,1′-Bis(diphenylphosphino) ferrocene]dichloropalladium (II); PIDA: (Diacetoxyiodo)benzene; prep TLC: preparative thin layer chromatography; q: quintet; RT: room temperature; Rt: retention time; s: singlet; t: triplet; THF: tetrahydrofuran; TLC: thin layer chromatography; UPLC: ultra performance liquid chromatography; UV: ultra-violet;

Other abbreviations are intended to convey their generally accepted meaning.

### General Experimental Conditions

All starting materials and solvents were obtained either from commercial sources or prepared according to literature methods. The appropriate isocyanate and aniline starting materials were either commercially available and were obtained from, for example, Sigma Aldrich, Fluorochem or Enamine store, or were synthesised as described herein. The appropriate tricyclic amine starting materials were synthesised as described herein. Reaction mixtures were magnetically stirred and reactions performed at room temperature (approximately 20° C.) unless otherwise indicated.

Silica gel chromatography was performed on an automated flash chromatography system, such as CombiFlash Companion, CombiFlash Rf system or Reveleris X2 flash system using RediSep® Rf or Reveleris® or the GraceResolv™ pre-packed silica (230-400 mesh, 40-63 μm) cartridges.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below. NMR spectra were recorded using either a Bruker Avance III HD 500 MHz instrument, a Bruker Avance Neo 400 MHz, Bruker Avance III 400 MHz instrument or a QOne AS400 400 MHz spectrometer using either residual non-deuterated solvent, or tetra-methylsilane as a reference.

In the absence of the absolute stereochemistry being explicitly indicated through wedged and dashed bonds, chemical structures disclosed throughout the examples (for example, in the configuration (1.3) described hereinabove) are to be interpreted as depicting the racemate. For the avoidance of doubt, the invention encompasses the compounds in either configuration, as well as mixtures thereof.

### Analytical Methods

Method 1 - Basic 3 min method

| Column: | Waters ACQUITY UPLC ® BEH C18, 1.7 μm, | | | |
|---|---|---|---|---|
| | 2.1 × 30 mm at 40° C. | | | |
| Detection: | UV at 210-400 nm unless otherwise | | | |
| | indicated, MS by electrospray ionisation | | | |
| Solvents: | A: 0.1% Ammonia in water, B: MeCN | | | |
| | | | | Flow rate |
| | Time | % A | % B | (ml/min) |
| Gradient: | 0.00 | 98 | 2 | 0.77 |
| | 2.50 | 0 | 100 | 0.77 |
| | 3.00 | 0 | 100 | 0.77 |

Method 2 - Acidic 3 min method 1

| Column: | Waters CSH C18, 1.7 μm, 2.1 × 30 mm at 40° C. | | | |
|---|---|---|---|---|
| Detection: | UV at 210-400 nm unless otherwise indicated, | | | |
| | MS by electrospray ionisation | | | |
| Solvents: | A: 0.1% formic acid in water, B: MeCN | | | |
| | Time | % A | % B | Flow rate (ml/min) |
| Gradient: | 0.00 | 98 | 2 | 0.77 |
| | 2.5 | 0 | 100 | 0.77 |
| | 3 | 0 | 100 | 0.77 |

Method 3 - Acidic 3 min method 2

| Column: | Waters CORTECS UPLC, C18, 1.6 μm, 2.1 × 50 mm | | | |
|---|---|---|---|---|
| | column at 25° C. | | | |
| Detection: | UV at 210-400 nm unless otherwise indicated, MS | | | |
| | by electrospray ionisation | | | |
| Solvents: | A: 0.1% formic acid in water, B: MeCN | | | |
| | Time | % A | % B | Flow rate (ml/min) |
| Gradient: | 0.00 | 80 | 20 | 0.6 |
| | 1.80 | 20 | 80 | 0.6 |
| | 2.65 | 20 | 80 | 0.6 |
| | 2.80 | 80 | 20 | 0.6 |
| | 3.00 | 80 | 20 | 0.6 |

Method 4 - Acidic 5 min method 2

| | |
|---|---|
| Column: | Waters Sunfire, 3.5 μm, 4.6 × 50 mm |
| | column at 25° C. |
| Detection: | UV at 214 and 254 nm unless otherwise |
| | indicated, MS by electrospray ionisation |
| Solvents: | A: 0.05% formic acid in water, |
| | B: 0.05% formic acid in MeCN |

| | Time | % A | % B | Flow rate (ml/min) |
|---|---|---|---|---|
| Gradient: | 0.00 | 85 | 15 | 1.0 |
| | 0.50 | 85 | 15 | 1.0 |
| | 4.00 | 0 | 100 | 1.0 |
| | 4.50 | 0 | 100 | 1.0 |
| | 4.51 | 85 | 15 | 1.0 |
| | 5.00 | 85 | 15 | 1.0 |

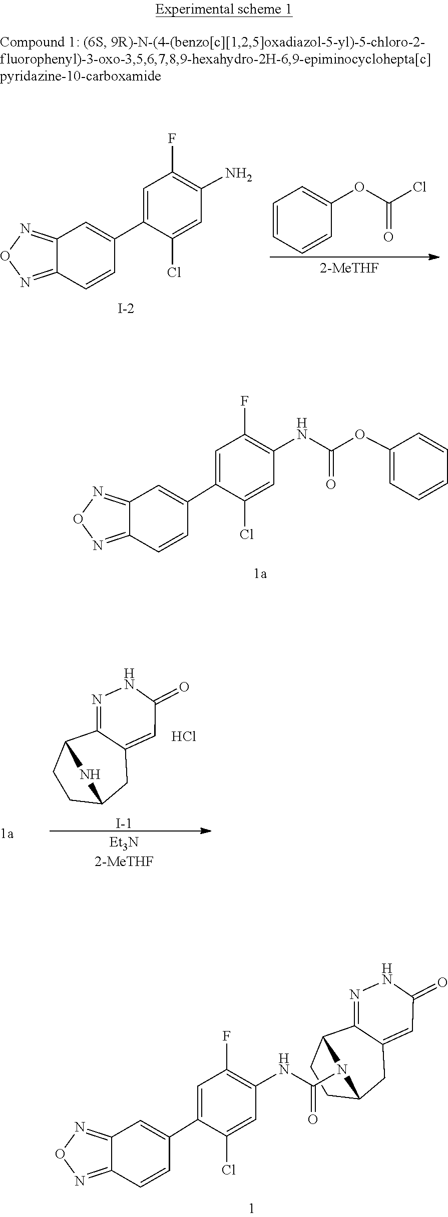

Step 1: A solution of phenyl chloroformate (428 μL, 3.41 mmol) in 2-MeTHF (1 mL) was slowly added to a solution of 4-(benzo[c][1,2,5]oxadiazol-5-yl)-5-chloro-2-fluoroaniline I-2 (1.00 g, 3.79 mmol) in 2-MeTHF (7 mL) at 0° C. The reaction was warmed to 40° C. for 3 h, before a further portion of phenyl chloroformate (80 μL, 379 μmol) in 2-MeTHF (0.1 mL) was added and the mixture was stirred at 40° C. for a further 20 h. The resulting mixture was used in the next step as obtained. A small portion of the reaction mixture was concentrated in vacuo and triturated from n-pentane and MTBE (1:1) to afford phenyl (4-(benzo[c][1,2,5]oxadiazol-5-yl)-5-chloro-2-fluorophenyl) carbamate as a brown solid. LCMS (Method 2) m/z 290.6 (M-OPh)⁻ (ES⁻), at 2.27 min, ¹H NMR (400 MHz, DMSO-d6) δ 10.42 (bs, 1H), 8.18 (s, 1H), 8.14 (d, J=9.3 Hz, 1H), 8.05 (d, J=7.0 Hz, 1H), 7.72 (d, J=9.2 Hz, 1H), 7.64 (d, J=11.0 Hz, 1H), 7.50-7.41 (m, 2H), 7.34-7.23 (m, 3H).
Step 2: To a suspension of the hydrochloride salt of (6S,9R)-2,5,6,7,8,9-hexahydro-3H-6,9-epiminocyclohepta[c]pyridazin-3-one I-1 (810 mg, 3.79 mmol) and Et₃N (2.11 mL, 15.2 mmol) in 2-MeTHF (5 mL) was added a solution of phenyl (4-(benzo[c][1,2,5]oxadiazol-5-yl)-5-chloro-2-fluorophenyl) carbamate (1.45 g, 3.79 mmol) in 2-MeTHF (8 ml) and the reaction was warmed to 40° C. for 3 h. The resulting mixture was cooled to RT and diluted with 2-MeTHF (10 mL), water (50 mL) was added and the layers were separated. The organics were washed with water (50 mL), brine (50 mL), dried with Na₂SO₄, and concentrated in vacuo. The product was purified by chromatography on silica gel (0-100% EtOAc: EtOH (3:1)/isohexane) to afford (6S,9R)—N-(4-(benzo[c][1,2,5]oxadiazol-5-yl)-5-chloro-2-fluorophenyl)-3-oxo-3,5,6,7,8,9-hexahydro-2H-6,9-epiminocyclohepta[c]pyridazine-10-carboxamide (1.56 g, 80%) as a tan solid. LCMS (Method 2) m/z 467.2, 469.2 (M+H)⁺ (ES⁺), at 1.70 min. ¹H NMR (400 MHz, DMSO-d6) δ 12.73 (bs, 1H), 8.97 (s, 1H), 8.16-8.09 (m, 2H), 7.86 (d, J=7.2 Hz, 1H), 7.73-7.66 (m, 1H), 7.54 (d, J=11.0 Hz, 1H), 6.69 (bs, 1H), 5.10 (d, J=5.6 Hz, 1H), 4.66 (t, J=6.2 Hz, 1H), 3.21 (dd, J=18.3, 5.3 Hz, 1H), 2.66 (d, J=18.2 Hz, 1H), 2.29-2.14 (m, 2H), 1.88-1.76 (m, 1H), 1.73-1.61 (m, 1H).

The following compounds could be prepared using appropriate starting materials in an analogous procedure to that described in Experimental Scheme 1. Where the starting materials are not described in the literature, their synthesis is described below.

| | | Method | |
|---|---|---|---|
| | | LCMS Rt | |
| Cpd | Structure and Name | [M + H]⁺ | NMR |
| 2 | 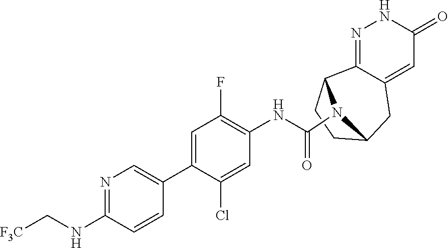 [SMILES: O=C(Nc1cc(Cl)c(-c2ccc(NCC(F)(F)F)nc2)cc1F)N1[C@H]2CC[C@@H]1c1n[nH]c(=O)cc1C2] | Method 1 523.2, 525.3 at 1.44 min | ¹H NMR (400 MHz, DMSO-d6) δ 12.72 (s, 1H), 8.87 (s, 1H), 8.11 (d, J = 2.4 Hz, 1H), 7.85 (s, 1H), 7.77-7.67 (m, 2H), 7.35 (d, J = 11.1 Hz, 1H), 6.86 (d, J = 8.8 Hz, 1H), 6.69 (s, 1H), 5.07 (d, J = 5.8 Hz, 1H), 4.64 (t, J = 6.5 Hz, 1H), 4.33- 4.22 (m, 2H), 3.23-3.19 (m, 1H), 2.68-2.61 (m, 1H), 2.25- 2.12 (m, 2H), 1.84-1.75 (m, 1H), 1.72-1.63 (m, 1H). |
| | (6S,9R)-N-(5-Chloro-2-fluoro-4-(6- | | |
| | ((2,2,2-trifluoroethyl)amino)pyridin-3- | | |
| | yl)phenyl)-3-oxo-3,5,6,7,8,9- | | |
| | hexahydro-2H-6,9-epimino- | | |
| | cyclohepta[c]pyridazine-10- | | |
| | carboxamide | | |
| 3 | 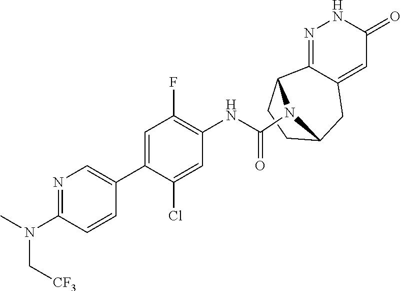 [SMILES: CN(CC(F)(F)F)c1ccc(-c2cc(F)c(NC(=O)N3[C@H]4CC[C@@H]3c3n[nH]c(=O)cc3C4)cc2Cl)cn1] | Method 1 537.3, 539.3 at 1.37 min | ¹H NMR (400 MHz, DMSO-d6) δ 12.72 (d, J = 2.2 Hz, 1H), 8.84 (s, 1H), 8.20 (d, J = 2.5 Hz, 1H), 7.75-7.68 (m, 2H), 7.34 (d, J = 11.1 Hz, 1H), 6.87 (d, J = 8.8 Hz, 1H), 6.69 (s, 1H), 5.07 (d, J = 5.7 Hz, 1H), 4.64 (d, J = 6.4 Hz, 1H), 4.53 (q, J = 9.6 Hz, 2H), 3.19 (dd, J = 18.4, 5.1 Hz, 1H), 3.13 (s, 3H), 2.65 (d, J = 18.1 Hz, 1H), 2.19 (dd, J = 11.7, 6.5 Hz, 2H), 1.86-1.75 (m, 1H), 1.73- 1.60 (m, 1H). |
| | (6S,9R)-N-(5-Chloro-2-fluoro-4-(6- | | |
| | (methyl(2,2,2-trifluoroethyl)- | | |
| | amino)pyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 4 | 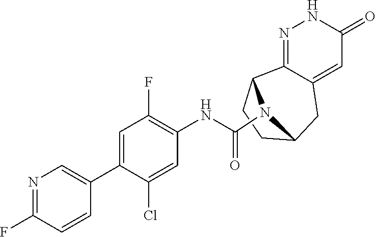 [SMILES: O=C(Nc1cc(Cl)c(-c2ccc(F)nc2)cc1F)N1[C@H]2CC[C@@H]1c1n[nH]c(=O)cc1C2] | Method 1 444.2, 446.2 at 1.04 min | ¹H NMR (400 MHz, DMSO-d6) δ 12.72 (d, J = 2.1 Hz, 1H), 8.92 (s, 1H), 8.31 (s, 1H), 8.10 (td, J = 8.2, 2.6 Hz, 1H), 7.81 (d, J = 7.3 Hz, 1H), 7.46 (d, J = 11.1 Hz, 1H), 7.30 (dd, J = 8.5, 2.8 Hz, 1H), 6.69 (d, J = 2.1 Hz, 1H), 5.08 (d, J = 5.7 Hz, 1H), 4.66-4.62 (m, 1H), 3.20 (dd, J = 18.4, 5.2 Hz, 1H), 2.66 (d, J = 18.1 Hz, 1H), 2.26- 2.14 (m, 2H), 1.85-1.76 (m, 1H), 1.73-1.64 (m, 1H). |
| | (6S,9R)-N-(5-Chloro-2-fluoro-4-(6- | | |
| | fluoropyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 5 | 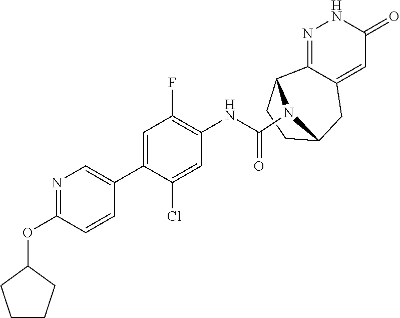 [SMILES: O=C(Nc1cc(Cl)c(-c2ccc(OC3CCCC3)nc2)cc1F)N1[C@H]2CC[C@@H]1c1n[nH]c(=O)cc1C2] | Method 1 510.3, 512.2 at 1.56 min | ¹H NMR (400 MHz, DMSO-d6) δ 12.72 (s, 1H), 8.87 (s, 1H), 8.20 (dd, J = 2.6, 0.8 Hz, 1H), 7.79-7.73 (m, 2H), 7.37 (d, J = 11.1 Hz, 1H), 6.83 (dd, J = 8.6, 0.7 Hz, 1H), 6.69 (s, 1H), 5.40 (dt, J = 6.0, 3.2 Hz, 1H), 5.07 (d, J = 5.8 Hz, 1H), 4.65 (d, J = 6.2 Hz, 1H), 3.20 (d, J = 14.7 Hz, 1H), 2.65 (d, J = 18.0 Hz, 1H), 2.29- 2.09 (m, 2H), 2.07-1.89 (m, 2H), 1.86-1.64 (m, 6H), 1.65- 1.51 (m, 2H). |
| | (6S,9R)-N-(5-Chloro-4-(6- | | |
| | (cyclopentyloxy)pyridin-3-yl)-2- | | |
| | fluorophenyl)-3-oxo-3,5,6,7,8,9- | | |
| | hexahydro-2H-6,9-epimino- | | |
| | cyclohepta[c]pyridazine-10- | | |
| | carboxamide | | |
| 6 | 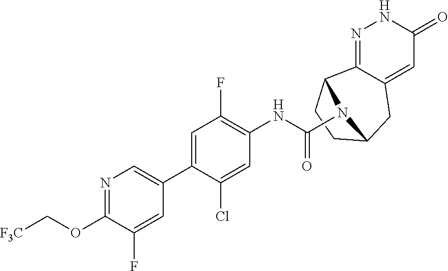 [SMILES: O=C(Nc1cc(Cl)c(-c2cnc(OCC(F)(F)F)c(F)c2)cc1F)N1[C@H]2CC[C@@H]1c1n[nH]c(=O)cc1C2] | Method 2 542.3, 544.4 at 1.88 min | ¹H NMR (400 MHz, DMSO-d6) δ 12.72 (s, 1H), 8.92 (s, 1H), 8.11 (d, J = 2.0 Hz, 1H), 8.00 (dd, J = 11.2, 2.0 Hz, 1H), 7.80 (d, J = 7.3 Hz, 1H), 7.45 (d, J = 11.0 Hz, 1H), 6.69 (s, 1H), 5.15 (q, J = 9.0 Hz, 2H), 5.10- 5.05 (m, 1H), 4.64 (t, J = 6.2 Hz, 1H), 3.23-3.16 (m, 1H), 2.71-2.61 (m, 1H), 2.19 (dd, J = 11.6, 6.4 Hz, 2H), 1.84- 1.77 (m, 1H), 1.73-1.64 (m, 1H). |
| | (6S,9R)-N-(5-Chloro-2-fluoro-4-(5- | | |
| | fluoro-6-(2,2,2-trifluoroethoxy)pyridin- | | |
| | 3-yl)phenyl)-3-oxo-3,5,6,7,8,9- | | |
| | hexahydro-2H-6,9-epimino- | | |
| | cyclohepta[c]pyridazine-10- | | |
| | carboxamide | | |
| 7 | 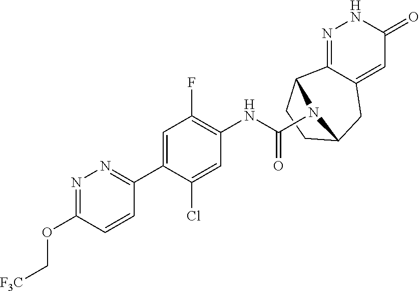 [SMILES: O=C(Nc1cc(Cl)c(-c2ccc(OCC(F)(F)F)nn2)cc1F)N1[C@H]2CC[C@@H]1c1n[nH]c(=O)cc1C2] | Method 4 525.1 at 1.50 min | ¹H NMR (400 MHz, DMSO- d6): δ 12.72 (s, 1H), 8.96 (s, 1H), 8.01 (d, J = 9.6 Hz, 1H), 7.85 (d, J = 7.2 Hz, 1H), 7.55 (s, 1H), 7.53 (d, J = 3.2 Hz, 1H), 6.69 (s, 1H), 5.25 (q, J = 9.6 Hz, 2H), 5.09 (d, J = 5.2 Hz, 1H), 4.66 (t, J = 5.6 Hz, 1H), 3.20 (dd, J = 5.6 Hz, J = 18.8 Hz, 1H), 2.65 (d, J = 18.0 Hz, 1H), 2.24-2.17 (m, 2H), 1.83-1.66 (m, 2H) |
| | (6S,9R)-N-(5-Chloro-2-fluoro-4-(6- | | |
| | hydroxypyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 8 | 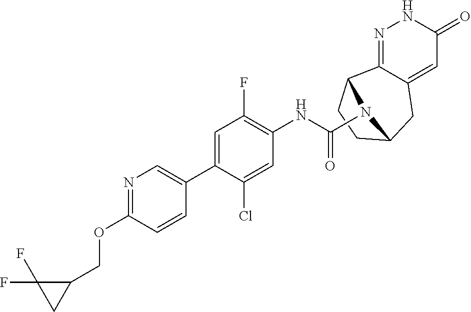 [SMILES: O=C(Nc1cc(Cl)c(-c2ccc(OCC3CC3(F)F)nc2)cc1F)N1[C@H]2CC[C@@H]1c1n[nH]c(=O)cc1C2] | Method 1 532.3, 534.4 at 1.39 min | ¹H NMR (400 MHz, DMSO-d6) δ 12.74 (s, 1H), 8.89 (s, 1H), 8.36-8.13 (m, 1H), 7.83 (dd, J = 8.6, 2.5 Hz, 1H), 7.76 (d, J = 7.4 Hz, 1H), 7.38 (d, J = 11.1 Hz, 1H), 7.04-6.86 (m, 1H), 6.69 (s, 1H), 5.07 (d, J = 5.7 Hz, 1H), 4.64 (s, 1H), 4.50 (ddd, J = 10.4, 6.6, 3.0 Hz, 1H), 4.24 (dd, J = 10.7, 8.3 Hz, 1H), 3.19 (d, J = 14.1 Hz, 1H), 2.65 (d, J = 18.3 Hz, 1H), 2.32- 2.05 (m, 3H), 1.87-1.63 (m, 3H), 1.63-1.45 (m, 1H). |
| | (6S,9R)-N-(5-Chloro-4-(6-((2,2- | | |
| | difluorocyclopropyl)methoxy)pyridin-3- | | |
| | yl)-2-fluorophenyl)-3-oxo-3,5,6,7,8,9- | | |
| | hexahydro-2H-6,9- | | |
| | epiminocyclohepta[c]pyridazine-10- | | |
| | carboxamide | | |
| 10 | 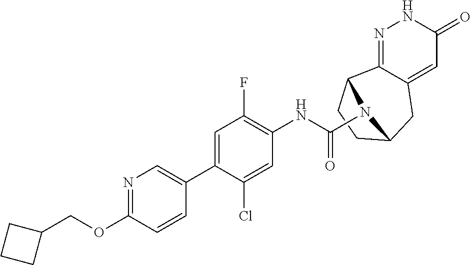 [SMILES: O=C(Nc1cc(Cl)c(-c2ccc(OCC3CCC3)nc2)cc1F)N1[C@H]2CC[C@@H]1c1n[nH]c(=O)cc1C2] | Method 2 510.3, 512.3 at 2.01 min | ¹H NMR (400 MHz, DMSO-d6) δ 12.72 (s, 1H), 8.87 (s, 1H), 8.20 (dd, J = 2.5, 0.8 Hz, 1H), 7.79 (dd, J = 8.6, 2.6 Hz, 1H), 7.76 (d, J = 7.4 Hz, 1H), 7.37 (d, J = 11.1 Hz, 1H), 6.88 (dd, J = 8.6, 0.8 Hz, 1H), 6.69 (s, 1H), 5.08 (d, J = 5.7 Hz, 1H), 4.64 (t, J = 6.0 Hz, 1H), 4.28 (d, J = 6.9 Hz, 2H), 3.20 (dd, J = 18.5, 5.3 Hz, 1H), 2.74 (p, J = 7.3 Hz, 1H), 2.65 (d, J = 18.2 Hz, 1H), 2.29-2.12 (m, 2H), 2.13-2.00 (m, 2H), 1.96- 1.76 (m, 5H), 1.72-1.63 (m, 1H). |
| | (6S,9R)-N-(5-Chloro-4-(6- | | |
| | (cyclobutylmethoxy)pyridin-3-yl)-2- | | |
| | fluorophenyl)-3-oxo-3,5,6,7,8,9- | | |
| | hexahydro-2H-6,9-epimino- | | |
| | cyclohepta[c]pyridazine-10- | | |
| | carboxamide | | |
| 12 | 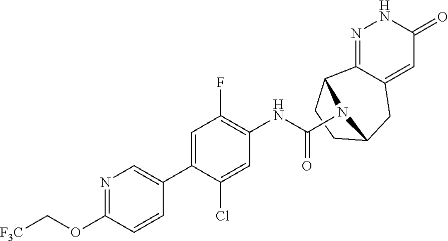 [SMILES: O=C(Nc1cc(Cl)c(-c2ccc(OCC(F)(F)F)nc2)cc1F)N1[C@H]2CC[C@@H]1c1n[nH]c(=O)cc1C2] | Method 1 524.4, 526.4 at 1.41 min | ¹H NMR (400 MHz, DMSO-d6) δ 12.72 (s, 1H), 8.89 (s, 1H), 8.27 (d, J = 2.3 Hz, 1H), 7.91 (dd, J = 8.6, 2.5 Hz, 1H), 7.78 (d, J = 7.3 Hz, 1H), 7.41 (d, J = 11.0 Hz, 1H), 7.08 (d, J = 8.6 Hz, 1H), 6.69 (s, 1H), 5.11- 4.99 (m, 3H), 4.64 (t, J = 5.9 Hz, 1H), 3.20 (dd, J = 18.3, 5.3 Hz, 1H), 2.65 (d, J = 18.1 Hz, 1H), 2.29-2.11 (m, 2H), 1.85- 1.75 (m, 1H), 1.74-1.64 (m, 1H). |
| | (6S,9R)-N-(5-Chloro-2-fluoro-4-(6- | | |
| | (2,2,2-trifluoroethoxy)pyridin-3- | | |
| | yl)phenyl)-3-oxo-3,5,6,7,8,9- | | |
| | hexahydro-2H-6,9-epimino- | | |
| | cyclohepta[c]pyridazine-10- | | |
| | carboxamide | | |
| 13 | 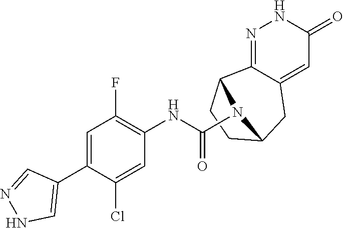 [SMILES: O=C(Nc1cc(Cl)c(-c2cn[nH]c2)cc1F)N1[C@H]2CC[C@@H]1c1n[nH]c(=O)cc1C2] | Method 1 415.3, 417.3 at 0.76 min | ¹H NMR (400 MHz, DMSO-d6) δ 12.72 (s, 1H), 8.77 (s, 1H), 8.37 (s, 1H), 7.66 (d, J = 7.5 Hz, 1H), 7.51 (d, J = 11.9 Hz, 1H), 6.68 (s, 1H), 5.05 (s, 1H), 4.63 (s, 1H), 3.17 (s, 1H), 2.53 (s, 1H), 2.19 (d, J = 6.5 Hz, 2H), 1.85-1.75 (m, 1H), 1.71- 1.45 (m, 1H), (2 exchangeable protons not observed) |
| | (6S,9R)-N-(5-Chloro-2-fluoro-4-(1H- | | |
| | pyrazol-4-yl)phenyl)-3-oxo-3,5,6,7,8,9- | | |
| | hexahydro-2H-6,9- | | |
| | epiminocyclohepta[c]pyridazine-10- | | |
| | carboxamide | | |
| 14 | 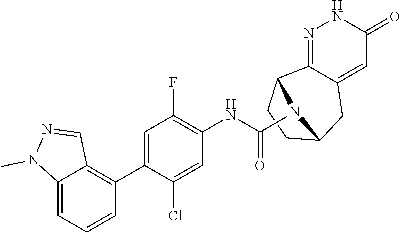 [SMILES: Cn1ncc2c(-c3cc(F)c(NC(=O)N4[C@H]5CC[C@@H]4c4n[nH]c(=O)cc4C5)cc3Cl)cccc21] | Method 1 479.2, 481.2 at 1.14 min | ¹H NMR (400 MHz, DMSO-d6) δ 12.73 (s, 1H), 8.92 (s, 1H), 7.84 (d, J = 7.3 Hz, 1H), 7.76 (d, J = 1.0 Hz, 1H), 7.70 (d, J = 8.5 Hz, 1H), 7.48 (dd, J = 8.5, 7.1 Hz, 1H), 7.37 (d, J = 11.0 Hz, 1H), 7.11 (d, J = 6.8 Hz, 1H), 6.70 (s, 1H), 5.10 (d, J = 5.8 Hz, 1H), 4.66 (s, 1H), 4.08 (s, 3H), 3.22 (d, J = 19.5 Hz, 1H), 2.66 (d, J = 18.3 Hz, 1H), 2.21 (s, 2H), 1.81 (t, J = 9.4 Hz, 1H), 1.70 (d, J = 12.0 Hz, 1H). |
| | (6S,9R)-N-(5-Chloro-2-fluoro-4-(1- | | |
| | methyl-1H-indazol-4-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 15 | 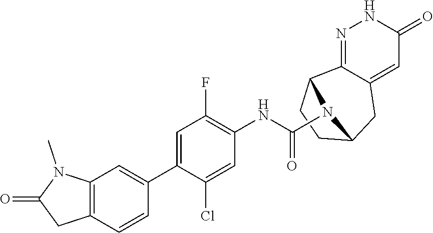 [SMILES: CN1C(=O)Cc2ccc(-c3cc(F)c(NC(=O)N4[C@H]5CC[C@@H]4c4n[nH]c(=O)cc4C5)cc3Cl)cc21] | Method 2 494.1, 496.1 at 1.58 min | ¹H NMR (400 MHz, DMSO-d6) δ 12.72 (s, 1H), 8.86 (s, 1H), 7.75 (d, J = 7.3 Hz, 1H), 7.36- 7.29 (m, 2H), 7.09-7.00 (m, 2H), 6.69 (s, 1H), 5.08 (d, J = 5.7 Hz, 1H), 4.65 (t, J = 5.6 Hz, 1H), 3.59 (s, 2H), 3.25- 3.16 (m, 1H), 3.13 (s, 3H), 2.66 (d, J = 18.2 Hz, 1H), 2.28- 2.12 (m, 2H), 1.80 (t, J = 9.6 Hz, 1H), 1.73-1.64 (m, 1H). |
| | (6S,9R)-N-(5-chloro-2-fluoro-4-(1- | | |
| | methyl-2-oxoindolin-6-yl)phenyl)-3- | | |
| | oxo-3,5,6,7,8,9-hexahydro-2H-6,9- | | |
| | epiminocyclohepta[c]pyridazine-10- | | |
| | carboxamide | | |
| 16 | 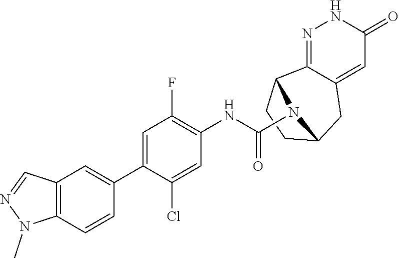 [SMILES: Cn1ncc2cc(-c3cc(F)c(NC(=O)N4[C@H]5CC[C@@H]4c4n[nH]c(=O)cc4C5)cc3Cl)ccc21] | Method 2 479.1, 481.0 at 1.66 min | ¹H NMR (400 MHz, DMSO-d6) δ 12.73 (s, 1H), 8.86 (s, 1H), 8.10 (d, J = 0.9 Hz, 1H), 7.80 (m, 1H), 7.75 (d, J = 7.3 Hz, 1H), 7.70 (dt, J = 8.8, 0.9 Hz, 1H), 7.45 (dd, J = 8.7, 1.6 Hz, 1H), 7.35 (d, J = 11.1 Hz, 1H), 6.69 (m, 1H), 5.08 (d, J = 5.6 Hz, 1H), 4.65 (t, J = 5.9 Hz, 1H), 4.08 (s, 3H), 3.21 (dd, J = 18.3, 5.3 Hz, 1H), 2.66 (d, J = 18.2 Hz, 1H), 2.26-2.12 (m, 2H), 1.88-1.76 (m, 1H), 1.73- 1.61 (m, 1H). |
| | (6S,9R)-N-(5-Chloro-2-fluoro-4-(1- | | |
| | methyl-1H-indazol-5-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 17 | 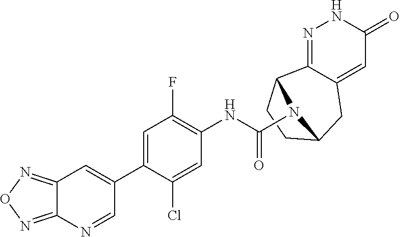 [SMILES: O=C(Nc1cc(Cl)c(-c2cnc3nonc3c2)cc1F)N1[C@H]2CC[C@@H]1c1n[nH]c(=O)cc1C2] | Method 2 468.0, 470.0 at 1.63 min | ¹H NMR (400 MHz, DMSO-d6) δ 12.73 (s, 1H), 9.19 (d, J = 2.1 Hz, 1H), 9.02 (s, 1H), 8.72 (d, J = 2.1 Hz, 1H), 7.91 (d, J = 7.2 Hz, 1H), 7.64 (d, J = 11.0 Hz, 1H), 6.70 (s, 1H), 5.10 (d, J = 5.6 Hz, 1H), 4.67 (t, J = 6.3 Hz, 1H), 3.21 (dd, J= 18.4, 5.4 Hz, 1H), 2.67 (d, J = 18.3 Hz, 1H), 2.27-2.13 (m, 2H), 1.89- 1.77 (m, 1H), 1.74-1.62 (m, 1H). |
| | (6S,9R)-N-(4-([1,2,5]Oxadiazolo[3,4- | | |
| | b]pyridin-6-yl)-5-chloro-2- | | |
| | fluorophenyl)-3-oxo-3,5,6,7,8,9- | | |
| | hexahydro-2H-6,9-epimin- | | |
| | ocyclohepta[c]pyridazine-10- | | |
| | carboxamide | | |
| 18 | 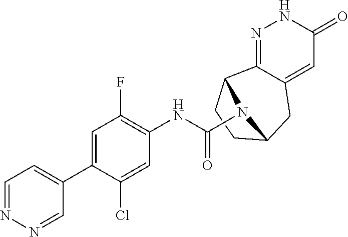 [SMILES: O=C(Nc1cc(Cl)c(-c2ccnnc2)cc1F)N1[C@H]2CC[C@@H]1c1n[nH]c(=O)cc1C2] | Method 1 427.3, 429.3 at 0.72 min | ¹H NMR (400 MHz, DMSO-d6) δ 12.73 (s, 1H), 9.37 (dd, J = 2.5, 1.2 Hz, 1H), 9.34 (dd, J = 5.3, 1.3 Hz, 1H), 8.99 (s, 1H), 7.89 (d, J = 7.2 Hz, 1H), 7.84 (dd, J = 5.3, 2.4 Hz, 1H), 7.58 (d, J = 11.1 Hz, 1H), 6.69 (s, 1H), 5.09 (d, J = 5.5 Hz, 1H), 4.66 (d, J = 6.4 Hz, 1H), 3.29- 3.11 (m, 1H), 2.66 (d, J = 18.2 Hz, 1H), 2.19 (dd, J = 11.6, 6.4 Hz, 2H), 1.87-1.75 (m, 1H), 1.68 (dd, J = 10.1, 6.2 Hz, 1H). |
| | (6S,9R)-N-(5-Chloro-2-fluoro-4- | | |
| | (pyridazin-4-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 19 | 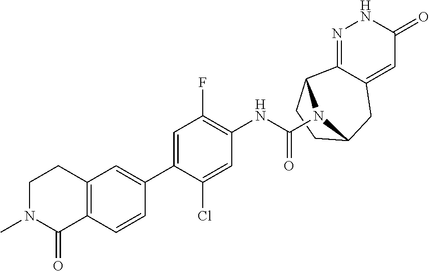 [SMILES: CN1CCc2cc(-c3cc(F)c(NC(=O)N4[C@H]5CC[C@@H]4c4n[nH]c(=O)cc4C5)cc3Cl)ccc2C1=O] | Method 1 508.5, 510.6 at 1.01 min | ¹H NMR (400 MHz, DMSO-d6) δ 12.72 (s, 1H), 8.89 (s, 1H), 7.92 (d, J = 8.0 Hz, 1H), 7.78 (d, J = 7.3 Hz, 1H), 7.40 (dd, J = 8.0, 1.8 Hz, 1H), 7.35 (d, J = 11.2 Hz, 2H), 6.69 (d, J = 1.5 Hz, 1H), 5.08 (d, J = 5.6 Hz, 1H), 4.65 (d, J = 6.0 Hz, 1H), 3.58 (t, J = 6.7 Hz, 2H), 3.32 (s, 4H), 3.26-3.14 (m, 1H), 3.02 (d, J = 6.6 Hz, 1H), 2.65 (d, J = 18.2 Hz, 1H), 2.29-2.10 (m, 2H), 1.86- 1.75 (m, 1H), 1.73-1.62 (m, 1H). |
| | (6S,9R)-N-(5-Chloro-2-fluoro-4-(2- | | |
| | methyl-1-oxo-1,2,3,4-tetrahydro- | | |
| | isoquinolin-6-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 20 | 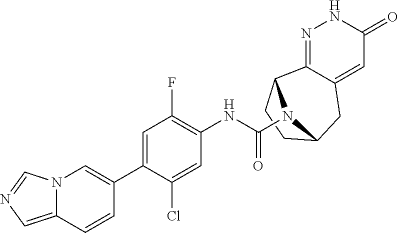 [SMILES: O=C(Nc1cc(Cl)c(-c2ccc3cncn3c2)cc1F)N1[C@H]2CC[C@@H]1c1n[nH]c(=O)cc1C2] | Method 2 464.9, 467.4 at 0.96 min | ¹H NMR (400 MHz, DMSO-d6) δ 8.46 (q, J = 1.2 Hz, 1H), 8.41 (d, J = 0.9 Hz, 1H), 7.81 (d, J = 7.3 Hz, 1H), 7.61 (d, J = 9.3 Hz, 1H), 7.44 (d, J = 11.1 Hz, 1H), 7.41 (s, 1H), 6.85 (dd, J = 9.3, 1.5 Hz, 1H), 6.69 (s, 1H), 5.09 (d, J = 5.6 Hz, 1H), 4.66 (s, 1H), 3.22 (d, J = 19.2 Hz, 1H), 2.75-2.62 (m, 1H), 2.21 (q, J = 7.2 Hz, 2H), 1.81 (t, J = 9.7 Hz, 1H), 1.70 (d, J = 13.3 Hz, 1H). |
| | (6S,9R)-N-(5-Chloro-2-fluoro-4- | | |
| | (imidazo[1,5-a]pyridin-6-yl)phenyl)-3- | | |
| | oxo-3,5,6,7,8,9-hexahydro-2H-6,9- | | |
| | epiminocyclohepta[c]pyridazine-10- | | |
| | carboxamide | | |
| 21 | 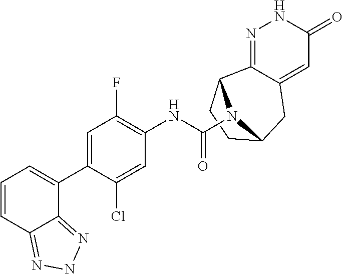 [SMILES: O=C(Nc1cc(Cl)c(-c2cccc3n[nH]nc23)cc1F)N1[C@H]2CC[C@@H]1c1n[nH]c(=O)cc1C2] | Method 1 467.0, 469.4 at 1.20 min | ¹H NMR (400 MHz, DMSO-d6) δ 8.12 (dd, J = 9.1, 0.8 Hz, 1H), 7.89 (d, J = 7.2 Hz, 1H), 7.73 (dd, J = 9.1, 6.7 Hz, 1H), 7.63 (dd, J = 6.7, 0.8 Hz, 1H), 7.56 (d, J = 11.1 Hz, 1H), 6.69 (s, 1H), 5.10 (d, J = 5.7 Hz, 1H), 4.67 (d, J = 6.3 Hz, 1H), 3.22 (dd, J = 18.2, 5.5 Hz, 1H), 2.66 (d, J = 18.1 Hz, 1H), 2.20 (dd, J = 11.5, 6.4 Hz, 2H), 1.81 (t, J = 9.5 Hz, 1H), 1.68 (t, J = 8.6 Hz, 1H). 2 exchangeable protons not observed |
| | (6S,9R)-N-(4-(Benzo[c][1,2,5]- | | |
| | oxadiazol-4-yl)-5-chloro-2- | | |
| | fluorophenyl)-3-oxo-3,5,6,7,8,9- | | |
| | hexahydro-2H-6,9-epimino- | | |
| | cyclohepta[c]pyridazine-10- | | |
| | carboxamide | | |
| 22 | 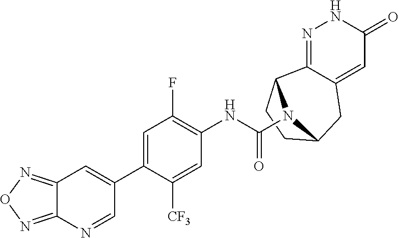 [SMILES: O=C(Nc1cc(C(F)(F)F)c(-c2cnc3nonc3c2)cc1F)N1[C@H]2CC[C@@H]1c1n[nH]c(=O)cc1C2] | Method 1 500.1, 501.3 at 1.69 min | ¹H NMR (400 MHz, DMSO-d6) δ 12.76 (s, 1H), 9.15 (s, 1H), 9.10-9.05 (m, 1H), 8.69 (d, J = 2.1 Hz, 1H), 8.19 (d, J = 7.4 Hz, 1H), 7.64 (d, J = 10.9 Hz, 1H), 6.73-6.68 (m, 1H), 5.12 (d, J = 5.6 Hz, 1H), 4.68 (s, 1H), 3.22 (dd, J = 18.1, 5.2 Hz, 1H), 3.16 (d, J = 5.1 Hz, 1H), 2.68 (d, J = 17.9 Hz, 1H), 2.21 (dd, J = 11.7, 6.5 Hz, 2H), 1.82 (t, J = 9.6 Hz, 1H), 1.71 (d, J = 11.8 Hz, 1H). |
| | (6S,9R)-N-(4-([1,2,5]Oxadiazolo[3,4- | | |
| | b]pyridin-6-yl)-2-fluoro-5- | | |
| | (trifluoromethyl)phenyl)-3-oxo- | | |
| | 3,5,6,7,8,9-hexahydro-2H-6,9- | | |
| | epiminocyclohepta[c]pyridazine-10- | | |
| | carboxamide | | |
| 23 | 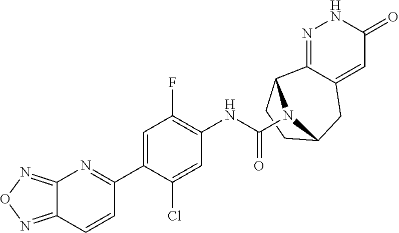 [SMILES: O=C(Nc1cc(Cl)c(-c2ccc3nonc3n2)cc1F)N1[C@H]2CC[C@@H]1c1n[nH]c(=O)cc1C2] | Method 1 468.1, 470.3 at 1.11 min | ¹H NMR (400 MHz, DMSO-d6) δ 12.76-12.71 (m, 1H), 9.07 (s, 1H), 8.69 (d, J = 9.3 Hz, 1H), 7.99 (d, J = 9.4 Hz, 1H), 7.96 (d, J = 7.1 Hz, 1H), 7.68 (d, J = 11.0 Hz, 1H), 6.73- 6.65 (m, 1H), 5.11 (d, J = 5.7 Hz, 1H), 4.68 (t, J = 6.0 Hz, 1H), 3.21 (dd, J = 18.3, 5.3 Hz, 1H), 2.67 (d, J = 17.9 Hz, 1H), 2.32-2.13 (m, 2H), 1.86- 1.77 (m, 1H), 1.74-1.62 (m, 1H). |
| | (6S,9R)-N-(4-([1,2,5]Oxadiazolo[3,4- | | |
| | b]pyridin-5-yl)-5-chloro-2- | | |
| | fluorophenyl)-3-oxo-3,5,6,7,8,9- | | |
| | hexahydro-2H-6,9-epimino- | | |
| | cyclohepta[c]pyridazine-10- | | |
| | carboxamide | | |
| 24 | 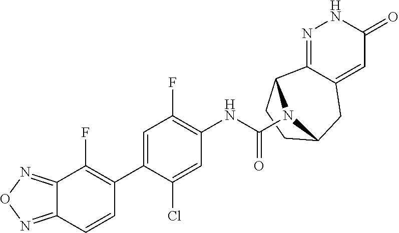 [SMILES: O=C(Nc1cc(Cl)c(-c2ccc3nonc3c2F)cc1F)N1[C@H]2CC[C@@H]1c1n[nH]c(=O)cc1C2] | Method 2 485.3, 487.3 at 1.73 min | ¹H NMR (400 MHz, DMSO-d6) δ 12.73 (d, J = 2.1 Hz, 1H), 9.00 (s, 1H), 8.04 (d, J = 9.2 Hz, 1H), 7.91 (d, J = 7.2 Hz, 1H), 7.64 (dd, J = 9.2, 6.3 Hz, 1H), 7.58 (d, J = 10.9 Hz, 1H), 6.69 (s, 1H), 5.10 (d, J = 5.6 Hz, 1H), 4.66 (t, J = 6.1 Hz, 1H), 3.21 (dd, J = 18.4, 5.6 Hz, 1H), 2.66 (d, J = 18.2 Hz, 1H), 2.29-2.12 (m, 2H), 1.88- 1.77 (m, 1H), 1.74-1.66 (m, 1H). |
| | (6S,9R)-N-(5-Chloro-2-fluoro-4-(4- | | |
| | fluorobenzo[c][1,2,5]oxadiazol-5- | | |
| | yl)phenyl)-3-oxo-3,5,6,7,8,9- | | |
| | hexahydro-2H-6,9-epimino- | | |
| | cyclohepta[c]pyridazine-10- | | |
| | carboxamide | | |

### Intermediate 1 (I-1) Route 1

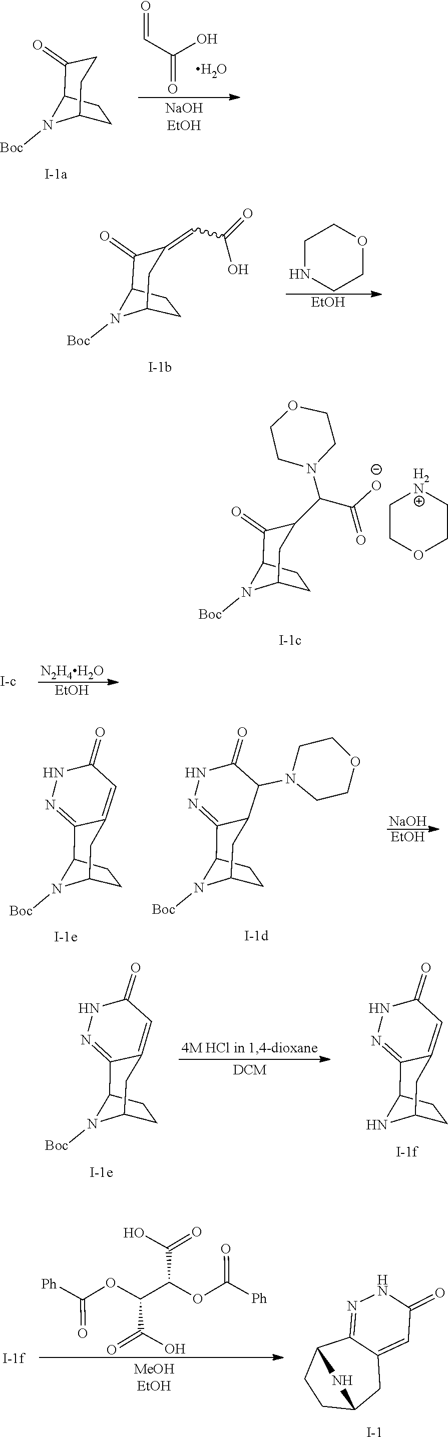

Step 1: To a solution of tert-butyl (+)-2-oxo-8-azabicyclo[3.2.1]octane-8-carboxylate I-1a (2.00 g, 8.88 mmol) and glyoxylic acid monohydrate (1.14 g, 12.4 mmol) in EtOH (20 ml) was added an aqueous 2 M sodium hydroxide solution (7.10 ml, 14.2 mmol). The resultant mixture was stirred at RT for 1 h and the reaction mixture was concentrated in vacuo. The aqueous was washed once with DCM (5 ml) before 1 M aq. HCl was carefully added to the remaining aqueous mixture until ~pH 6 was reached. The product was extracted with DCM (100 ml) and the aqueous layer was further extracted with 10% MeOH/DCM (3×50 ml) and the combined organic layers were dried over MgSO₄, filtered and concentrated to provide 2-((±)-8-(tert-butoxycarbonyl)-2-oxo-8-azabicyclo[3.2.1]octa n-3-ylidene) acetic acid I-1b as an off white solid. ¹H NMR (400 MHz, DMSO-d6) δ 6.64-6.54 (m, 1H), 5.75 (s, 1H), 4.35 (d, J=8.1 Hz, 1H), 3.32 (s, 1H), 3.06 (t, J=2.6 Hz, 1H), 2.32-2.16 (m, 1H), 2.16-1.96 (m, 1H), 1.79-1.51 (m, 2H), 1.37 (s, 9H). (Exchangeable —OH not observed)
Step 2: To a solution of 2-((±)-8-(tert-butoxycarbonyl)-2-oxo-8-azabicyclo[3.2.1]octan-3-ylidene) acetic acid I-1b (4.1 g, 13 mmol) in EtOH (30 ml) at 0° C. was added morpholine (2.3 ml, 27 mmol) dropwise. The reaction was stirred at this temperature for 1 h, then allowed to warm to RT and stirred for 72 h. The mixture was concentrated in vacuo to give 2-((±)-8-(tert-butoxycarbonyl)-2-oxo-8-azabicyclo[3.2.1]octan-3-yl)-2-morpholinoacetic acid, morpholine salt I-1c as a sticky yellow oil that was used in the next step without any further purification or analysis.
Step 3: To a solution of 2-((±)-8-(tert-butoxycarbonyl)-2-oxo-8-azabicyclo[3.2.1]octan-3-yl)-2-morpholinoacetic acid, morpholine salt I-1c (17.02 g, 37.37 mmol) in EtOH (120 ml) was added hydrazine monohydrate in water (11.3 ml, 149.5 mmol). The resultant mixture was heated to 78° C. for 2.5 h, cooled to RT and then the mixture was concentrated in vacuo. The resultant yellow residue was dissolved in DCM (500 ml) and water (150 ml). The layers were separated, and the aqueous layer was extracted with 10% MeOH in DCM (3×200 ml). The combined organic layers were dried over MgSO₄ and concentrated in vacuo to provide a pale yellow solid. The solid was dissolved in the minimum amount of EtOH and allowed to sit at RT for 16 h and the resulting precipitate was isolated by filtration to provide tert-butyl (±)-3-oxo-3,5,6,7,8,9-hexahydro-2H-6,9-epiminocyclohepta[c]pyridazine-10-carboxylate I-1e as a white powder. The filtrate was concentrated to provide a mixture of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1d as a sticky brown oil.
I-1e: ¹H NMR (400 MHz, DMSO-d6) δ 6.67 (s, 1H), 4.71 (d, J=6.2 Hz, 1H), 4.31 (br s, 1H), 3.03 (d, J=17.9 Hz, 1H), 2.61 (d, J=18.1 Hz, 1H), 2.22-2.07 (m, 2H), 1.73 (t, J=9.6 Hz, 1H), 1.60 (t, J=8.1 Hz, 1H), 1.35 (s, 9H). (Exchangeable proton not visible in spectrum)
I-1d: ¹H NMR (400 MHz, DMSO-d6) δ 10.48 (s, 1H), 4.42 (d, J=6.9 Hz, 1H), 4.22 (d, J=6.7 Hz, 1H), 3.55-3.48 (m, 4H), 3.09-2.92 (m, 4H), 2.69 (dd, J=12.1, 6.0 Hz, 2H), 2.06-2.00 (m, 2H), 1.92 (d, J=10.1 Hz, 1H), 1.69-1.65 (m, 2H), 1.56 (d, J=12.7 Hz, 1H), 1.38 (s, 9H).
Step 4: To a solution of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1d (3.2 g, 66% Mol. Wt I-1d) in EtOH (40 ml) was added a 2 M NaOH solution (10 ml, 20 mmol) and the resultant mixture was stirred at RT for 16 h. The reaction mixture was concentrated in vacuo and the aqueous was neutralised to pH 7 using 1 M HCl. The product was extracted with 10% MeOH in DCM (3×150 ml) and the combined organics were dried with MgSO₄. The solvent was removed in vacuo to give tert-butyl (±)-3-oxo-3,5,6,7,8,9-hexahydro-2H-6,9-epiminocyclohepta[c]pyridazine-10-carboxylate I-1e as a white solid.
Step 5: To a solution of tert-butyl (±)-3-oxo-3,5,6,7,8,9-hexahydro-2H-6,9-epiminocyclohepta[c]pyridazine-10-carboxylate (2.6 g, 9.4 mmol) in DCM (20 ml) was added a solution of HCl in 1,4-dioxane (23 ml, 4 M, 94 mmol). The reaction was stirred at RT for 72 h and then concentrated in vacuo to give the HCl salt of 1-1f. The HCl salt was dissolved in MeOH (150 ml), AcOH was added and the solution was loaded onto SCX resin (50 g). The cartridge was washed with MeOH and the product was eluted with 0.7 M NH₃ in MeOH solution. To give (±)-2,5,6,7,8,9-hexahydro-3H-6,9-epiminocyclohepta[c]pyridazin-3-one I-1f as an off white solid.

Alternatively, the HCl salt was dissolved in MeOH and MP-carbonate (3 eq.) was added. The mixture was left for 1 h before the resin was isolated by filtration and washed with MeOH. The filtrate was concentrated in vacuo to give (+)-2,5,6,7,8,9-hexahydro-3H-6,9-epiminocyclohepta[c]pyridazin-3-one I-1f as an off white solid.

### Step 6

To a solution of (±)-2,5,6,7,8,9-hexahydro-3H-6,9-epiminocyclohepta[c]pyridazin-3-one I-1f (200 mg, 1.13 mmol) in MeOH (10 ml) was added a solution of dibenzoyl-L-tartaric acid in MeOH (5 ml). The solvent was removed in vacuo and the residue was redissolved in EtOH (40 ml), heated to 60° C. for 1 h and allowed to cool and left standing for 2 h. The solid was filtered off and dried in vacuo and then dissolved in MeOH (10 ml) and MP-carbonate (600 mg, 1.8 mmol) was added and the mixture was left to stand for 1 h. The solid was filtered and the filtrate concentrated in vacuo to give (5R,8S)-3,5,6,7,8,9-hexahydro-2H-5,8-epiminocyclohepta[d]pyrimidin-2-one I-1 as an off white solid. ¹H NMR (400 MHz, DMSO-d6) δ 12.48 (s, 1H), 6.56 (s, 1H), 4.05-3.99 (m, 1H), 3.63 (t, J=5.9 Hz, 1H), 2.90-2.78 (m, 1H), 2.45 (t, J=1.4 Hz, 1H), 1.98-1.81 (m, 2H), 1.65 (t, J=9.3 Hz, 1H), 1.53-1.39 (m, 1H). (NH not observed).

### Intermediate 2 (I-32)

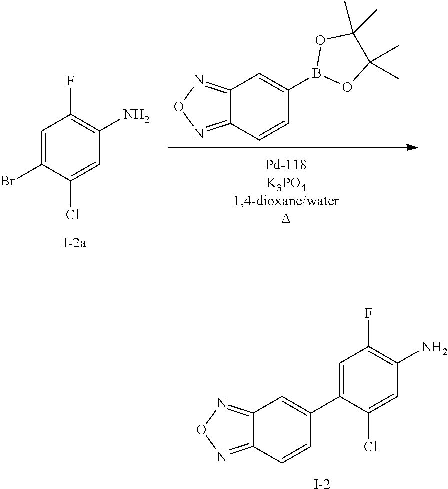

To a vial was added 4-bromo-5-chloro-2-fluoroaniline I-2a (316 mg, 1.41 mmol), 5-(4,4,5,5-tetramethyl-1,3,2-dioxaborolan-2-yl)benzo[c][1,2,5]oxadiazole (346 mg, 1.41 mmol) and Pd-118 (22.9 mg, 35.2 μmol). The vial was evacuated under vacuum and back filled with N₂, with the process being repeated 3 times. 1,4-Dioxane (4.00 ml) was added, followed by an aqueous solution of tripotassium phosphate (2.11 ml, 2.00 M, 4.22 mmol) which had been sparged with N₂. After the addition was complete, the vial was once more evacuated and backfilled with N₂. The reaction was heated to 95° C. for 2 h and then allowed to cool to RT and filtered through a pad of Celite, washing with DCM (20 ml). The filtrate was diluted with water (4 ml) and the layers were separated. The aqueous phase was extracted with DCM (3×10 ml). The combined organics were dried over MgSO₄, filtered and concentrated in vacuo. The product was purified by chromatography on silica gel (0-30% EtOAc/isohexane) to afford 4-(benzo[c][1,2,5]oxadiazol-5-yl)-5-chloro-2-fluoroaniline I-2 as a bright yellow solid. ¹H NMR (400 MHz, DMSO-d6) δ 8.06 (dd, J=9.3, 1.0 Hz, 1H), 8.00 (t, J=1.3 Hz, 1H), 7.68 (dd, J=9.3, 1.4 Hz, 1H), 7.31 (d, J=11.9 Hz, 1H), 6.95 (d, J=8.2 Hz, 1H), 5.81 (s, 2H).

### Intermediate 3 (I-3)

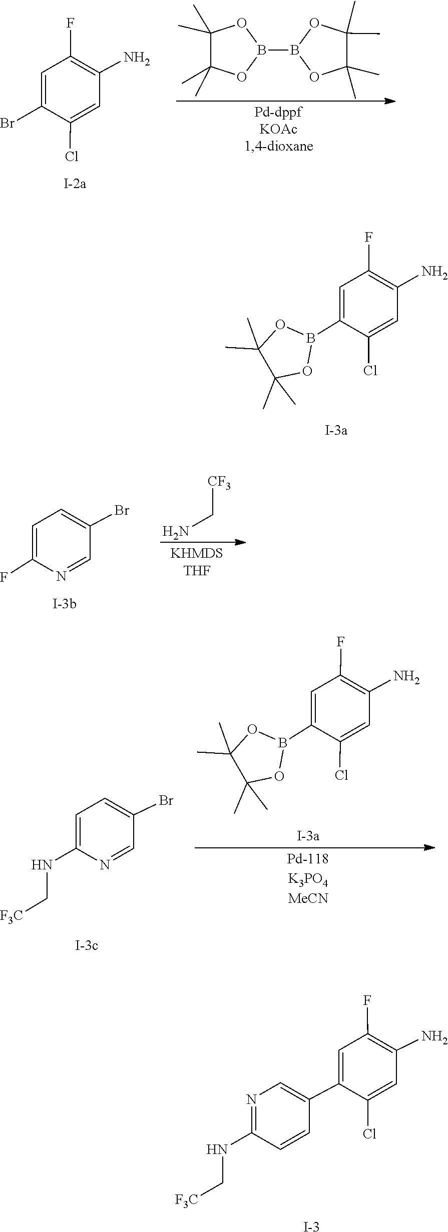

Step 1: To a solution of 4-bromo-5-chloro-2-fluoroaniline I-2a (10.0 g, 44.6 mmol), potassium acetate (13.1 g, 134 mmol) and bis(pinacolato)diboron (17.0 g, 66.8 mmol) in 1,4-dioxane (100 mL) was added Pd(dppf)Cl₂ (2.73 g, 3.34 mmol). The reaction was heated to 100° C. for 4 h, before cooling to RT and stirring for 20 h. The resulting mixture was diluted with EtOAc (200 mL) before being filtered. The precipitate was washed with additional EtOAc (200 mL) and the filtrate was concentrated in vacuo. The precipitate was partitioned between DCM (150 mL) and saturated aqueous NaHCO₃ (150 mL) and the organics were washed with saturated aqueous NaHCO₃ (150 mL) and brine (150 mL) and concentrated in vacuo. The residue was suspended in pentane (300 mL), stirred for 45 minutes and then cooled to 0° C. before being filtered. The precipitate was washed with additional pentane (2×50 mL) and dried in vacuo to afford 5-chloro-2-fluoro-4-(4,4,5,5-tetramethyl-1,3,2-dioxaborolan-2-yl)-benzenamine I-3a as a black powder. ¹H NMR (400 MHz, DMSO-d6) δ 7.14 (d, J=11.9 Hz, 1H), 6.73 (d, J=7.7 Hz, 1H), 5.89 (s, 2H), 1.16 (s, 12H)
Step 2: To a solution of 2,2,2-trifluoroethylamine (674 μL, 8.52 mmol) and 5-bromo-2-fluoro-pyridine I-3b (585 μL, 5.68 mmol) in THF (3 mL) was slowly added a solution of KHMDS in THF (8.5 mL, 1 M, 8.52 mmol). The reaction was stirred at RT for 1 h then quenched with MeOH (10 mL) and the mixture was concentrated in vacuo. The product was purified by chromatography on silica gel (0-30% EtOAc/isohexane) to afford 5-bromo-N-(2,2,2-trifluoroethyl)pyridin-2-amine I-3c as a sticky white solid. ¹H NMR (400 MHz, DMSO-d6) δ 8.09 (d, J=2.5 Hz, 1H), 7.62 (dd, J=8.9, 2.5 Hz, 1H), 7.38 (t, J=6.6 Hz, 1H), 6.62 (d, J=8.8 Hz, 1H), 4.13 (qd, J=9.8, 6.6 Hz, 2H).
Step 3: To a suspension of 5-bromo-N-(2,2,2-trifluoroethyl)pyridin-2-amine I-3c (250 mg, 980 μmol), 5-chloro-2-fluoro-4-(4,4,5,5-tetramethyl-1,3,2-dioxaborolan-2-yl) aniline I-3a (505 mg, 1.08 mmol) and Pd-118 (32 mg, 49.0 μmol) in MeCN (4 mL) under N₂ atmosphere was added a pre-degassed aqueous solution of potassium phosphate tribasic (1.47 mL, 2 M, 2.94 mmol). The reaction was heated to 77° C. for 2 h and then cooled to RT before being poured into brine (40 mL), followed by extraction with EtOAc (2×40 mL). The combined organics were washed with brine (30 mL), dried over magnesium sulphate, filtered and concentrated in vacuo. The material was purified by chromatography on silica gel (0-30% EtOAc/isohexane) to afford 5-(4-amino-2-chloro-5-fluorophenyl)-N-(2,2,2-trifluoroethyl)pyridin-2-amine I-3 as a tan solid. ¹H NMR (400 MHz, DMSO-d6) δ 8.01 (dd, J=2.4, 0.8 Hz, 1H), 7.49 (dd, J=8.6, 2.4 Hz, 1H), 7.25 (t, J=6.6 Hz, 1H), 7.05 (d, J=12.0 Hz, 1H), 6.88 (d, J=8.4 Hz, 1H), 6.65 (dd, J=8.7, 0.8 Hz, 1H), 5.50 (s, 2H), 4.18 (qd, J=9.8, 6.5 Hz, 2H).

### Intermediate 4 (I-4)

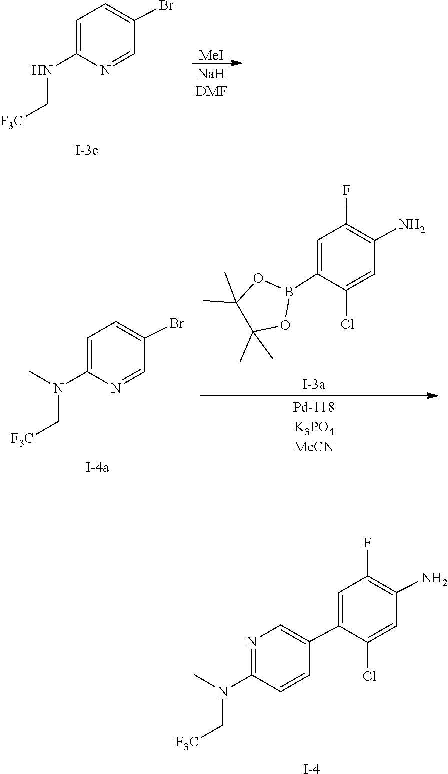

Step 1: To a solution of 5-bromo-N-(2,2,2-trifluoroethyl)pyridin-2-amine I-3c (200 mg, 784 μmol) in DMF (1.00 mL) at 0° C. was added NaH (38 mg, 60% Wt, 941 μmol) in a single portion. Mel (98 μL, 1.57 mmol) was added and stirring was continued at 0° C. for 30 min. The reaction was quenched with water (1 mL) and the product was extracted into EtOAc (3×5 mL). The combined organics were dried over MgSO₄, filtered and concentrated in vacuo. The product was purified by chromatography on silica gel (0-15% EtOAc/isohexane) to afford 5-bromo-N-methyl-N-(2,2,2-trifluoroethyl)pyridin-2-amine I-4a as a colourless oil. ¹H NMR (400 MHz, DMSO-d6) δ 8.20 (dd, J=2.6, 0.7 Hz, 1H), 7.76 (dd, J=9.0, 2.6 Hz, 1H), 6.79 (dd, J=9.1, 0.7 Hz, 1H), 4.45 (q, J=9.6 Hz, 2H), 3.07 (d, J=1.1 Hz, 3H).
Step 2: 5-(4-Amino-2-chloro-5-fluorophenyl)-N-methyl-N-(2,2,2-trifluoroethyl)pyridin-2-amine I-4 was synthesised from 5-bromo-N-methyl-N-(2,2,2-trifluoroethyl)pyridin-2-amine I-4a and 5-chloro-2-fluoro-4-(4,4,5,5-tetramethyl-1,3,2-dioxaborolan-2-yl) aniline I-3a using a procedure essentially the same as for 1-3. ¹H NMR (400 MHz, DMSO-d6) δ 8.13 (dd, J=2.4, 0.8 Hz, 1H), 7.63 (dd, J=8.8, 2.5 Hz, 1H), 7.08 (d, J=12.0 Hz, 1H), 6.89 (d, J=8.4 Hz, 1H), 6.82 (dd, J=8.9, 0.9 Hz, 1H), 5.52 (d, J=3.3 Hz, 2H), 4.50 (q, J=9.6 Hz, 2H), 3.11 (s, 3H).

### Intermediate 5 (I-5)

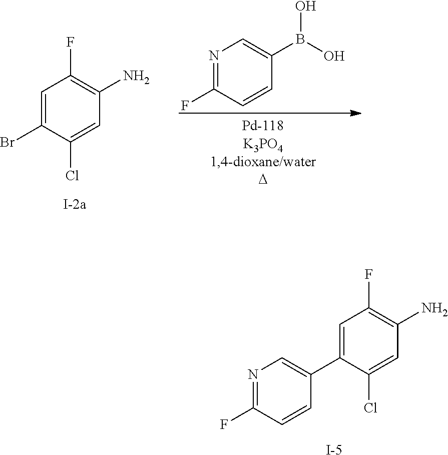

A flask containing 4-bromo-5-chloro-2-fluoroaniline I-2a (50.0 g, 222.8 mmol) and (6-fluoropyridin-3-yl) boronic acid (31.4 g, 222.8 mmol) was purged with N₂ for 5 min before MeCN (500 mL) was added and N₂ was bubbled through the reaction mixture. Pd-118 (3.63 g, 5.57 mmol) was added followed by an aqueous solution of tripotassium phosphate (334 mL, 2 M, 668.3 mmol). The resulting mixture was purged with N₂ for 5 min, heated to 90° C., stirred for 2 h and then allowed to cool to RT before being left to stir for 16 h, at which time a precipitate had formed. This was filtered off and washed with acetonitrile (50 mL), water (50 mL) and DCM (200 mL) and dried in vacuo to give 5-chloro-2-fluoro-4-(6-fluoropyridin-3-yl) aniline I-5 as a brown solid. ¹H NMR (400 MHz, DMSO-d6) δ 8.23 (d, J=2.6 Hz, 1H), 8.02 (td, J=8.2, 2.6 Hz, 1H), 7.27-7.16 (m, 2H), 6.93 (d, J=8.3 Hz, 1H), 5.69 (s, 2H).

### Intermediate 6

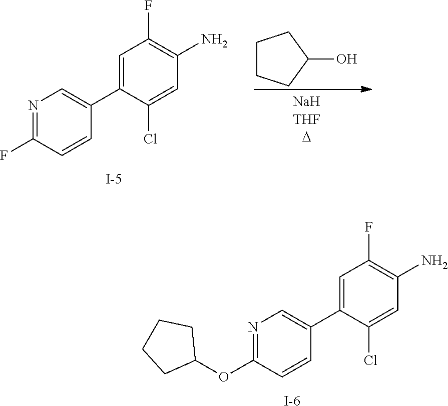

To a solution of cyclopentanol (107 mg, 1.25 mmol) in THF (3 mL) at 0° C. was added sodium hydride (53 mg, 60% wt., 1.33 mmol) and the resulting mixture was stirred at RT for 30 min before a solution of 5-chloro-2-fluoro-4-(6-fluoropyridin-3-yl) aniline 1-5 (200 mg, 831 μmol) in THF (3 mL) was added. The resulting mixture was heated to 60° C. for 2.5 h, before the being allowed to cool to RT and water (0.1 mL), brine (25 mL) and DCM (25 mL) were added. The layers were separated and the aqueous was extracted with DCM (25 mL) before the combined organics were dried with MgSO₄ and concentrated in vacuo. The product was purified by chromatography on silica gel (0-20% EtOAc/isohexane) to afford 5-chloro-4-(6-(cyclopentyloxy)pyridin-3-yl)-2-fluoroaniline I-6, as a sticky yellow oil. ¹H NMR (400 MHz, DMSO-d6) δ 8.13 (dd, J=2.6, 0.7 Hz, 1H), 7.69 (dd, J=8.6, 2.5 Hz, 1H), 7.11 (d, J=11.9 Hz, 1H), 6.90 (d, J=8.3 Hz, 1H), 6.77 (dd, J=8.5, 0.7 Hz, 1H), 5.58 (s, 2H), 5.48-5.31 (m, 1H), 1.99-1.86 (m, 2H), 1.82-1.66 (m, 4H), 1.66-1.45 (m, 2H).

### Intermediate 7 (I-7)

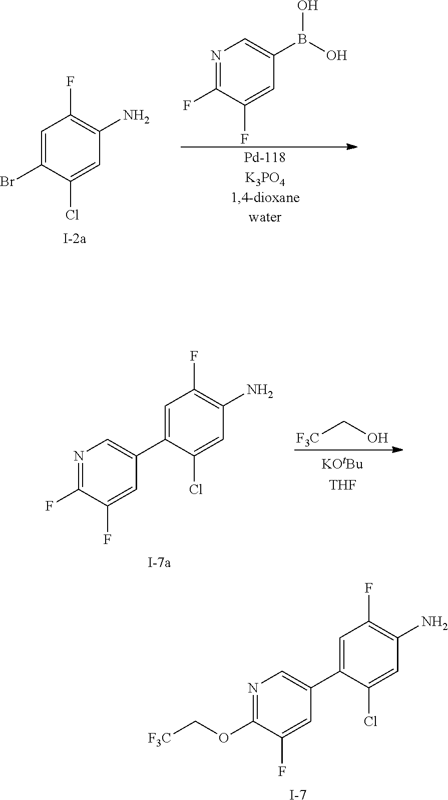

Step 1: A vial containing 4-bromo-5-chloro-2-fluoroaniline I-2a (424 mg, 1.89 mmol), (5,6-difluoropyridin-3-yl) boronic acid (250 mg, 1.57 mmol) and Pd-118 (29 mg, 44.9 μmol) was flushed with N₂, before 1,4-dioxane (3 mL) was added, followed by a degassed aqueous solution of potassium phosphate (2.36 mL, 2 M, 4.72 mmol). The reaction mixture was heated to 95° C. for 24 h, allowed to cool to RT and water (15 mL) was added and the product was extracted into DCM (3×10 mL). The combined organics were passed through a hydrophobic frit and the filtrate was concentrated in vacuo. The product was purified by chromatography on silica gel (30-100% DCM/heptane), and a subsequent trituration of material from n-pentane gave 5-chloro-4-(5,6-difluoropyridin-3-yl)-2-fluoroaniline I-7a as a light yellow solid. ¹H NMR (400 MHz, DMSO-d6) δ 8.14 (ddd, J=10.8, 9.3, 2.1 Hz, 1H), 8.08 (t, J=1.9 Hz, 1H), 7.24 (d, J=11.9 Hz, 1H), 6.93 (d, J=8.3 Hz, 1H), 5.76 (s, 2H)
Step 2: To a mixture of 5-chloro-4-(5,6-difluoropyridin-3-yl)-2-fluoroaniline I-7a (300 mg, 1.10 mmol) and trifluoroethanol (320 μL, 4.41 mmol) in THF (10 mL) at 0° C., was added a solution of KO Bu in THF (2.66 mL, 20% Wt, 4.41 mmol) and the resulting mass was allowed to warm to RT for 20 h. The reaction mixture was partitioned between brine (20 mL) and DCM (20 mL) and the layers were separated. The aqueous was extracted into DCM (20 mL), the organics were combined and concentrated in vacuo. The product was purified by chromatography on silica gel (0-20% (0.7 M ammonia/MeOH)/DCM) to afford 5-chloro-2-fluoro-4-(5-fluoro-6-(2,2,2-trifluoroethoxy)pyridin-3-yl) aniline I-7 as a pale yellow oil. ¹H NMR (400 MHz, DMSO-d6) δ 8.04 (d, J=1.9 Hz, 1H), 7.89 (dd, J=11.3, 2.0 Hz, 1H), 7.20 (d, J=11.9 Hz, 1H), 6.92 (d, J=8.3 Hz, 1H), 5.69 (s, 2H), 5.12 (q, J=9.0 Hz, 2H).

### Intermediate 8 (I-8)

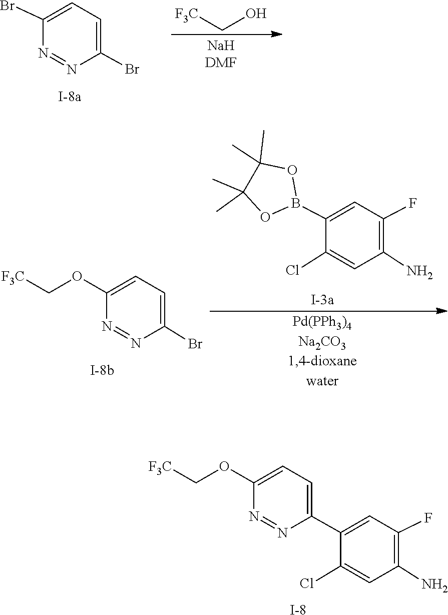

Step 1: To a solution of trifluoroethanol (305 μL, 4.24 mmol) in DMF (10 mL) was added NaH (339 mg, 8.48 mmol) at 0° C. After stirring at 0° C. for 30 min, 3,6-dibromopyridazine I-8a (1 g, 4.24 mmol) was added and the reaction was stirred at RT for 16 h. The mixture was poured into aqueous NH₄Cl solution (5 mL) and extracted into EtOAc (3×30 mL). The combined organics were washed with brine (80 mL), dried over Na₂SO₄ and concentrated in vacuo. The product was purified by chromatography on silica gel (10% EtOAc/petroleum ether) to give 3-bromo-6-(2,2,2-trifluoroethoxy) pyridazine I-8b as a light yellow solid. ¹H NMR (400 MHz, DMSO-d6) δ 8.00 (d, J=9.2 Hz, 1H), 7.45 (d, J=9.2 Hz, 1H), 5.15 (q, J=8.8 Hz, 2H).
Step 2: A mixture of 5-chloro-2-fluoro-4-(4,4,5,5-tetramethyl-1,3,2-dioxaborolan-2-yl) aniline I-3a (300 mg, 1.10 mmol), 3-bromo-6-(2,2,2-trifluoroethoxy) pyridazine I-8b (284 mg, 1.1 mmol), Na₂CO₃ (352 mg, 3.30 mmol) and Pd(PPh₃)₄ (128 mg, 0.11 mmol) in 1,4-dioxane (10 mL) and water (2 mL) was heated to 100° C. for 16 h. The reaction was allowed to cool to RT and concentrated in vacuo. The product was purified by chromatography on silica gel (10% EtOAc/petroleum ether) to give 5-chloro-2-fluoro-4-(6-(2,2,2-trifluoroethoxy) pyridazin-3-yl) aniline 1-8 as a white solid. LCMS (Method 3) m/z 322.0 (M+H)⁺ (ES⁺), at 1.39 min

### Intermediate 9 (I-9)

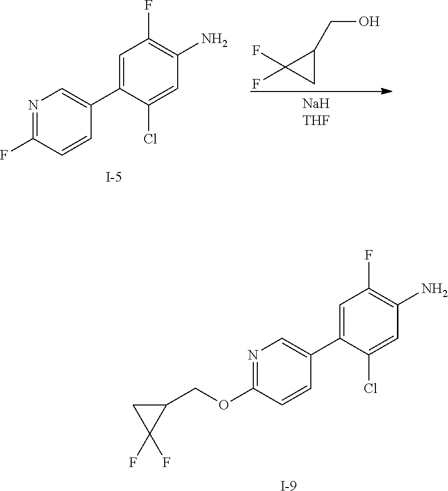

5-Chloro-4-(6-((2,2-difluorocyclopropyl) methoxy)pyridin-3-yl)-2-fluoroaniline I-9 was synthesised from 5-chloro-2-fluoro-4-(6-fluoropyridin-3-yl) aniline 1-5 and (2,2-difluorocyclopropyl) methanol using a procedure essentially the same as for 1-6. ¹H NMR (400 MHz, DMSO-d6) δ 8.14 (dd, J=2.6, 0.8 Hz, 1H), 7.75 (dd, J=8.6, 2.5 Hz, 1H), 7.12 (d, J=11.9 Hz, 1H), 6.99-6.78 (m, 2H), 5.59 (s, 2H), 4.61-4.39 (m, 1H), 4.22 (ddd, J=11.7, 8.6, 1.8 Hz, 1H), 2.36-2.14 (m, 1H), 1.79-1.71 (m, 1H), 1.53-1.46 (m, 1H).

### Intermediate 12 (I-12)

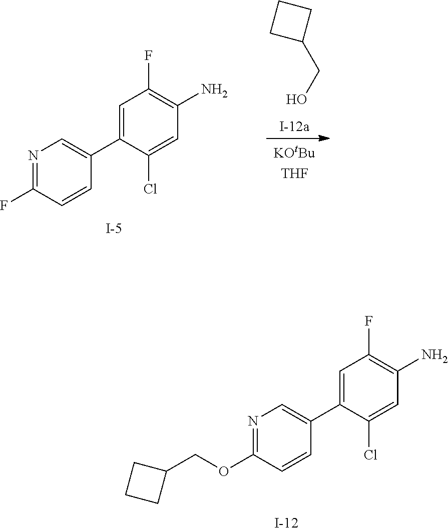

5-Chloro-4-(6-(cyclobutylmethoxy)pyridin-3-yl)-2-fluoroaniline I-12 was synthesised from 5-chloro-2-fluoro-4-(6-fluoropyridin-3-yl) aniline I-5 and cyclobutylmethanol I-12a using a procedure essentially the same as for 1-10. ¹H NMR (400 MHz, DMSO-d6) δ 8.12 (dd, J=2.5, 0.7 Hz, 1H), 7.71 (dd, J=8.6, 2.5 Hz, 1H), 7.12 (d, J=11.9 Hz, 1H), 6.90 (d, J=8.4 Hz, 1H), 6.83 (dd, J=8.6, 0.7 Hz, 1H), 5.63-5.57 (m, 2H), 4.25 (d, J=7.0 Hz, 2H), 2.81-2.67 (m, 1H), 2.13-2.01 (m, 2H), 1.96-1.79 (m, 4H).

### Intermediate 14 (I-14)

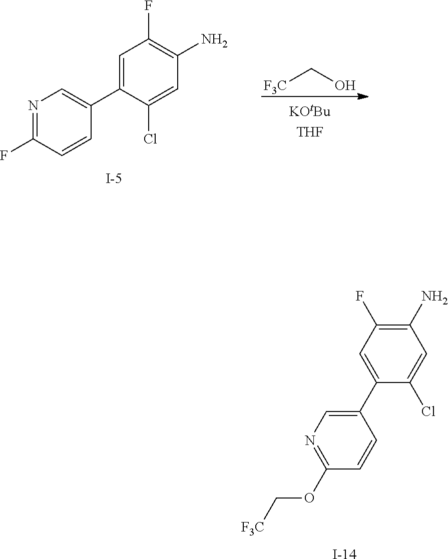

Step 1:5-Chloro-2-fluoro-4-(6-(2,2,2-trifluoroethoxy)pyridin-3-yl) aniline I-14 was synthesised from 5-chloro-2-fluoro-4-(6-fluoropyridin-3-yl) aniline I-5 using a procedure essentially the same as for I-10. ¹H NMR (400 MHz, DMSO-d₆) δ 8.19 (dd, J=2.5, 0.8 Hz, 1H), 7.83 (dd, J=8.6, 2.5 Hz, 1H), 7.15 (d, J=11.9 Hz, 1H), 7.03 (dd, J=8.5, 0.7 Hz, 1H), 6.92 (d, J=8.3 Hz, 1H), 5.63 (s, 2H), 5.02 (q, J=9.1 Hz, 2H).

### Intermediate 15 (I-15)

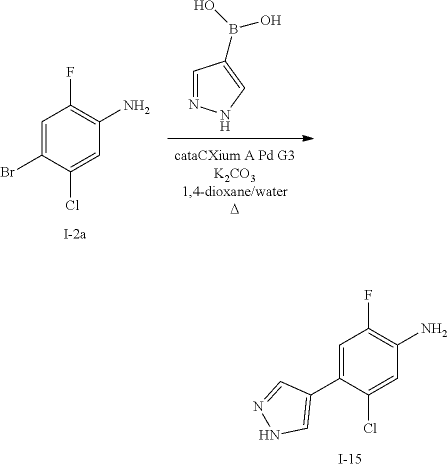

To a vial containg cataCXium® A Pd G3 (3.2 mg, 4.5 μmol) was added (1H-pyrazol-4-yl) boronic acid (29.9 mg, 267 μmol) and 4-bromo-5-chloro-2-fluoroaniline I-2a (20.0 mg, 89.1 μmol) in 1,4-dioxane (1.00 ml) and a solution of K₂CO₃ in water (208 μl, 1.5 M, 312 μmol). The mixture was stirred at 95° C. for 2 h and then loaded onto SCX (1 g), washed with MeOH and the product was eluted with 0.7 M ammonia in MeOH to afford 5-chloro-2-fluoro-4-(1H-pyrazol-4-yl) aniline I-15. LCMS (Method 1) m/z 212.2, 214.2 (M+H)⁺ (ES⁺), at 0.85 min.

### Intermediate 16 (I-16)

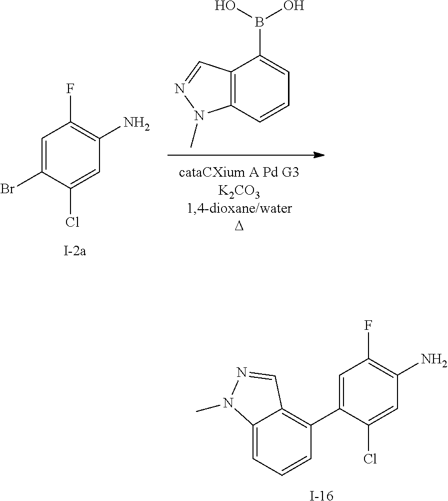

5-Chloro-2-fluoro-4-(1-methyl-1H-indazol-4-yl) aniline I-16 was synthesised from 4-bromo-5-chloro-2-fluoroaniline I-2a using a procedure essentially the same as for I-15. LCMS (Method 1) m/z 275.8 (M+H)⁺ (ES⁺), at 1.40 min

### Intermediate 17 (I-17)

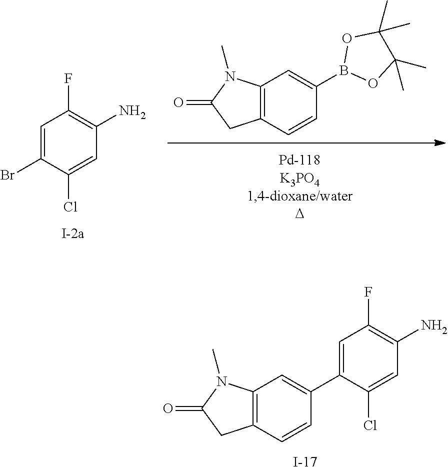

6-(4-Amino-2-chloro-5-fluorophenyl)-1-methylindolin-2-one I-17 was synthesised from 4-bromo-5-chloro-2-fluoroaniline I-2a and 1-methyl-6-(4,4,5,5-tetramethyl-1,3,2-dioxaborolan-2-yl) indolin-2-one using a procedure essentially the same as for I-7a. ¹H NMR (400 MHz, DMSO-d6) δ 7.27 (d, J=7.6 Hz, 1H), 7.09 (d, J=11.9 Hz, 1H), 7.01 (dd, J=7.6, 1.6 Hz, 1H), 6.95 (d, J=1.5 Hz, 1H), 6.90 (d, J=8.4 Hz, 1H), 5.56 (s, 2H), 3.56 (d, J=1.1 Hz, 2H), 3.13 (s, 3H).

### Intermediate 18 (I-18)

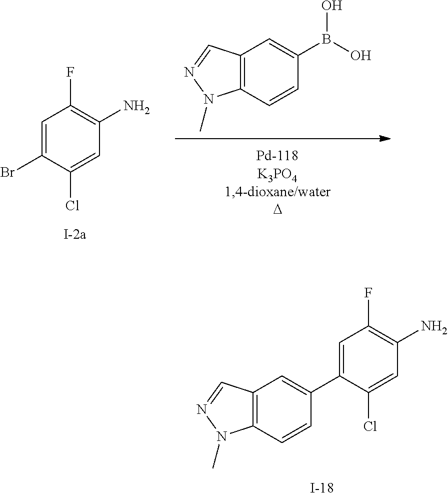

5-Chloro-2-fluoro-4-(1-methyl-1H-indazol-5-yl) aniline I-18 was synthesised from 4-bromo-5-chloro-2-fluoroaniline I-2a and (1-methyl-1H-indazol-5-yl) boronic acid using a procedure essentially the same as for I-7a. ¹H NMR (400 MHz, DMSO-d6) δ 8.05 (d, J=0.9 Hz, 1H), 7.73-7.68 (m, 1H), 7.67-7.62 (m, 1H), 7.40 (dd, J=8.7, 1.6 Hz, 1H), 7.10 (d, J=11.9 Hz, 1H), 6.91 (d, J=8.4 Hz, 1H), 5.52 (s, 2H), 4.06 (s, 3H).

### Intermediate 19 (I-19)

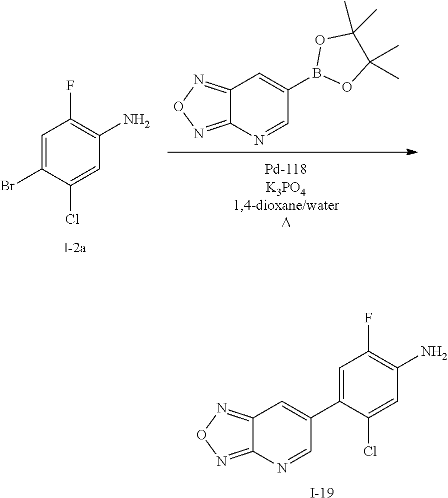

4-([1,2,5]Oxadiazolo[3,4-b]pyridin-6-yl)-5-chloro-2-fluoroaniline I-19 was synthesised from 4-bromo-5-chloro-2-fluoroaniline I-2a and 6-(4,4,5,5-tetramethyl-1,3,2-dioxaborolan-2-yl)-[1,2,5]oxadiazolo[3,4-b]pyridine using a procedure essentially the same as for I-7a. ¹H NMR (400 MHz, DMSO-d6) δ 9.15 (d, J=2.1 Hz, 1H), 8.55 (d, J=2.1 Hz, 1H), 7.42 (d, J=11.9 Hz, 1H), 6.98 (d, J=8.2 Hz, 1H), 5.95 (s, 2H).

### Intermediate 20 (I-20)

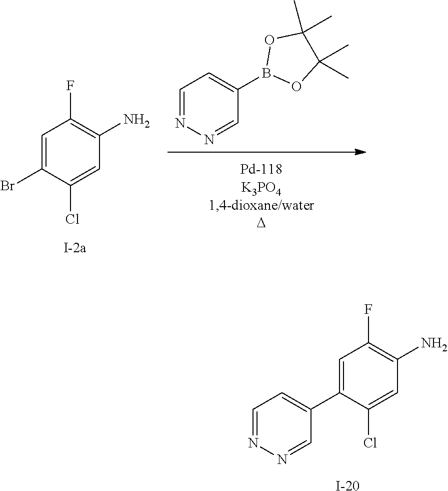

5-Chloro-2-fluoro-4-(pyridazin-4-yl) aniline I-20 was synthesised from 4-bromo-5-chloro-2-fluoroaniline I-2a and 4-(4,4,5,5-tetramethyl-1,3,2-dioxaborolan-2-yl) pyridazine using a procedure essentially the same as for I-7a. ¹H NMR (400 MHz, DMSO-d6) δ 9.31 (dd, J=2.5, 1.2 Hz, 1H), 9.24 (dd, J=5.4, 1.2 Hz, 1H), 7.76 (dd, J=5.4, 2.4 Hz, 1H), 7.36 (d, J=12.0 Hz, 1H), 6.95 (d, J=8.2 Hz, 1H), 5.92 (s, 2H).

### Intermediate 21 (I-21)

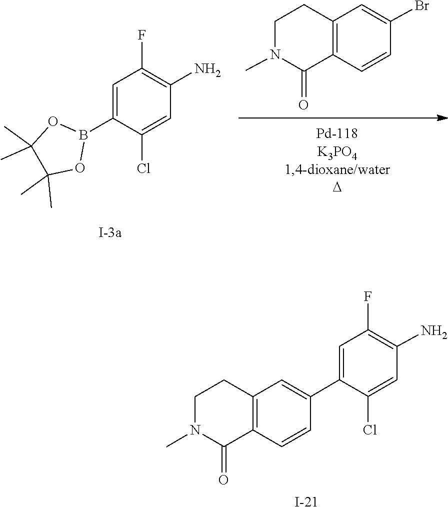

6-bromo-2-methyl-3,4-dihydroisoquinolin-1-one (141 mg, 589 μmol), 5-chloro-2-fluoro-4-(4,4,5,5-tetramethyl-1,3,2-dioxaborolan-2-yl) aniline I-3a (160 mg, 589 μmol) and Pd-118 (10.0 mg, 15.3 μmol) were added to a scintillation vial. The vial was flushed with N₂ before 1,4-dioxane (3.00 ml) was added, followed by a degassed aqueous solution of potassium phosphate (884 μl, 2 M, 1.77 mmol). The reaction was heated to 95° C. for 2 h before being allowed to cool to RT and water (15 ml) was added followed by extraction with DCM (3×10 ml). The combined organics were passed through a hydrophobic frit and concentrated in vacuo. The product was purified by chromatography on silica gel (10-30% EtOAc/DCM) to afford 6-(4-amino-2-chloro-5-fluorophenyl)-2-methyl-3,4-dihydroisoquinolin-1 (2H)-one 1-21 as a light grey solid. ¹H NMR (400 MHz, DMSO-d6) δ 7.87 (d, J=8.0 Hz, 1H), 7.35 (dd, J=8.0, 1.8 Hz, 1H), 7.30 (d, J=1.7 Hz, 1H), 7.11 (d, J=11.9 Hz, 1H), 6.91 (d, J=8.3 Hz, 1H), 5.63 (s, 2H), 3.56 (t, J=6.7 Hz, 2H), 3.03 (s, 3H), 3.00 (t, J=6.7 Hz, 2H).

### Intermediate 22 (I-22)

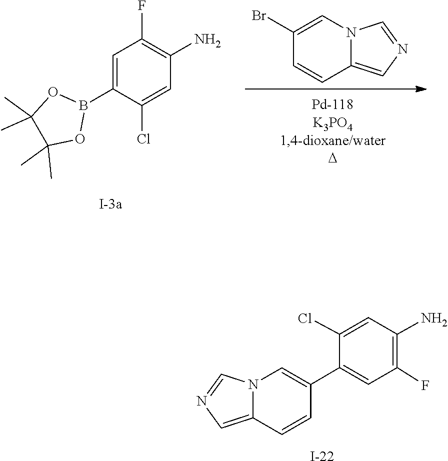

5-Chloro-2-fluoro-4-(imidazo[1,5-a]pyridine-6-yl) aniline I-22 was synthesised from 5-chloro-2-fluoro-4-(4,4,5,5-tetramethyl-1,3,2-dioxaborolan-2-yl) aniline I-3a and 6-bromoimidazo[1,5-a]pyridine using a procedure essentially the same as for I-21. ¹H NMR (400 MHz, DMSO-d6) δ 8.41-8.32 (m, 2H), 7.55 (dt, J=9.4, 1.0 Hz, 1H), 7.37 (s, 1H), 7.19 (d, J=11.9 Hz, 1H), 6.92 (d, J=8.3 Hz, 1H), 6.81 (dd, J=9.4, 1.5 Hz, 1H), 5.63 (s, 2H).

### Intermediate 23 (I-23)

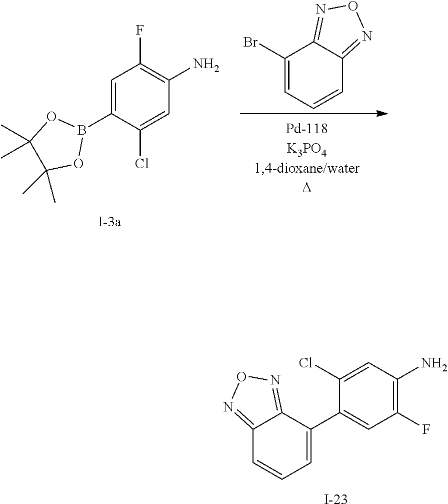

4-(Benzo[c][1,2,5]oxadiazol-4-yl)-5-chloro-2-fluoroaniline I-23 was synthesised from 5-chloro-2-fluoro-4-(4,4,5,5-tetramethyl-1,3,2-dioxaborolan-2-yl) aniline I-3a and 4-bromobenzo[c][1,2,5]oxadiazole using a procedure essentially the same as for I-21. ¹H NMR (400 MHz, DMSO-d6) δ 8.03 (dd, J=9.1, 0.8 Hz, 1H), 7.68 (dd, J=9.0, 6.7 Hz, 1H), 7.54 (dd, J=6.7, 0.8 Hz, 1H), 7.33 (d, J=11.9 Hz, 1H), 6.97 (d, J=8.3 Hz, 1H), 5.82 (s, 2H).

### Intermediate 24 (I-24)

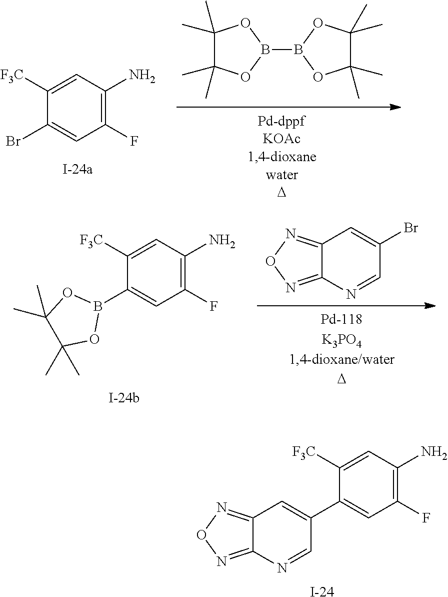

Step 1:2-Fluoro-4-(4,4,5,5-tetramethyl-1,3,2-dioxaborolan-2-yl)-5-(trifluoromethyl) aniline I-24b was synthesised from 4-bromo-2-fluoro-5-(trifluoromethyl) aniline I-24a using a procedure essentially the same as for I-3a. ¹H NMR (400 MHz, DMSO-d6) δ 7.28 (d, J=11.8 Hz, 1H), 7.14 (dd, J=8.4, 6.5 Hz, 1H), 5.97 (s, 2H), 1.25 (s, 12H).
Step 2:4-([1,2,5]Oxadiazolo[3,4-b]pyridin-6-yl)-2-fluoro-5-(trifluoromethyl) aniline I-24 was synthesised from 2-fluoro-4-(4,4,5,5-tetramethyl-1,3,2-dioxaborolan-2-yl)-5-(trifluoromethyl) aniline I-24b and 6-bromo-[1,2,5]oxadiazolo[3,4-b]pyridine using a procedure essentially the same as for I-21. ¹H NMR (400 MHz, DMSO-d6) δ 9.01 (dd, J=2.1, 0.9 Hz, 1H), 8.54 (d, J=2.1 Hz, 1H), 7.38 (d, J=11.8 Hz, 1H), 7.29 (d, J=8.5 Hz, 1H), 6.05 (s, 2H)

### Intermediate 25 (I-25)

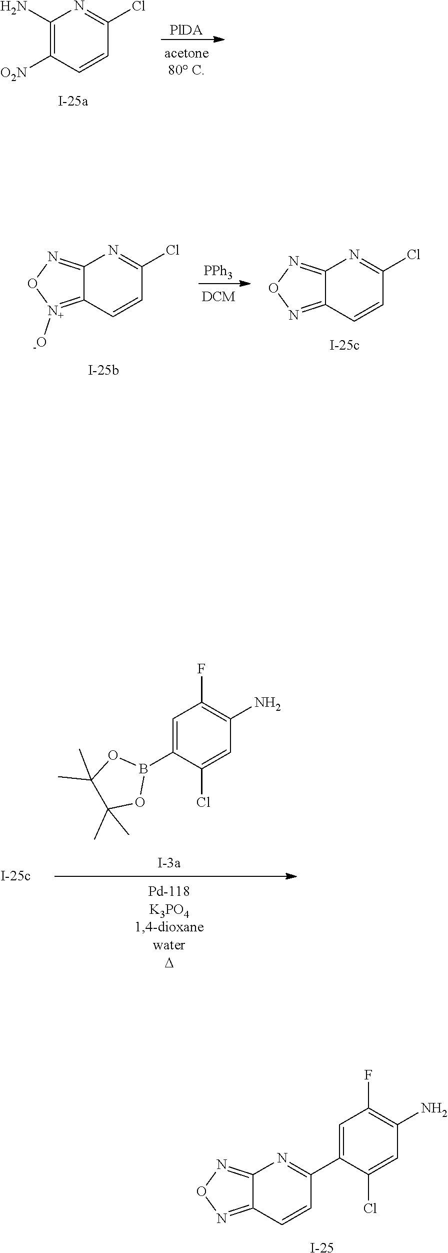

Step 1: To a solution of 6-chloro-3-nitropyridin-2-ylamine I-25a (1.00 g, 5.76 mmol) in acetone (60 ml) was added PIDA (4.64 g, 14.4 mmol). The reaction mixture was heated to 80° C. for 6 h and then concentrated in vacuo. The product was purified by chromatography on silica gel (0-100% EtOAc/DCM) to afford 5-chloro-[1,2,5]oxadiazolo[3,4-b]pyridine 1-oxide I-25b as a yellow solid. ¹H NMR (400 MHz, CDCl₃) δ 7.77 (d, J=9.1 Hz, 1H), 7.16 (d, J=9.2 Hz, 1H).
Step 2: A solution of 5-chloro-[1,2,5]oxadiazolo[3,4-b]pyridine 1-oxide I-25b (403 mg, 2.35 mmol) in DCM (60 ml) was cooled to 0° C. and triphenylphosphine (924 mg, 3.52 mmol) was added. The reaction mixture was warmed to RT and stirred for 16 h before a 1M aqueous NaOH solution (30 ml) was added and the mixture was vigorously stirred for 15 min. The product was extracted into DCM (3×50 ml) and the combined organics were dried over sodium sulphate and concentrated in vacuo. The product was purified by chromatography on silica gel (0-50% EtOAc/isohexane) and then further purified by chromatography on silica gel (0-30% EtOAc/isohexane) to afford 5-chloro-[1,2,5]oxadiazolo[3,4-b]pyridine I-25c as a yellow oil that solidified upon standing. ¹H NMR (400 MHz, CDCl₃) δ 8.22 (d, J=9.2 Hz, 1H), 7.40 (d, J=9.2 Hz, 1H)
Step 3:4-([1,2,5]Oxadiazolo[3,4-b]pyridin-5-yl)-5-chloro-2-fluoroaniline I-25 was synthesised from 5-chloro-2-fluoro-4-(4,4,5,5-tetramethyl-1,3,2-dioxaborolan-2-yl) aniline I-3a and 5-chloro-[1,2,5]oxadiazolo[3,4-b]pyridine I-25c using a procedure essentially the same as for 1-21. ¹H NMR (400 MHz, DMSO-d6) δ 8.57 (d, J=9.4 Hz, 1H), 7.99 (d, J=9.4 Hz, 1H), 7.53 (d, J=12.1 Hz, 1H), 6.95 (d, J=8.0 Hz, 1H), 6.21 (s, 2H).

### Intermediate 26 (I-26)

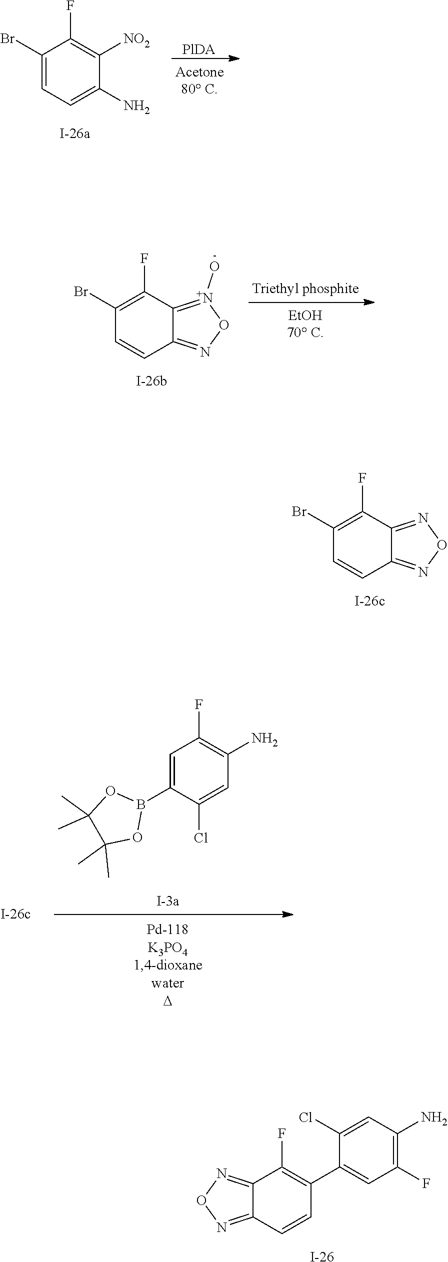

Step 1:5-Bromo-4-fluorobenzo[c][1,2,5]oxadiazole 1-oxide I-26b was synthesised from 4-bromo-3-fluoro-2-nitroaniline I-26a and PIDA using a procedure essentially the same as for I-25b. ¹H NMR (400 MHz, CDCl₃) δ 7.28-7.22 (m, 1H), 7.14 (d, J=9.5 Hz, 1H).
Step 2: To a solution of 5-bromo-4-fluorobenzo[c][1,2,5]oxadiazole 1-oxide I-26b (584 mg, 2.51 mmol) in EtOH (3 mL) was added triethyl phosphite (646 μL, 3.76 mmol) and the reaction mixture was heated to 77° C. for 2 h. The reaction was allowed to cool to RT, diluted with DCM (15 mL) and vigorously stirred for 20 min, after which a diluted 10% v/v sodium hypochlorite aqueous solution (10 mL) was added, the layers were separated and the organics were passed through a hydrophobic frit. The filtrate was concentrated in vacuo and the product was purified by chromatography on silica gel (0-40% EtOAc/iso-hexane) to afford 5-bromo-4-fluorobenzo[c][1,2,5]oxadiazole I-26c as an orange solid. ¹H NMR (400 MHz, DMSO-d6) δ7.95 (d, J=9.5 Hz, 1H), 7.82 (dd, J=9.4, 6.0 Hz, 1H).
Step 3:5-chloro-2-fluoro-4-(4-fluorobenzo[c][1,2,5]oxadiazol-5-yl) aniline I-26 was synthesised from 5-chloro-2-fluoro-4-(4,4,5,5-tetramethyl-1,3,2-dioxaborolan-2-yl) aniline I-3a and 5-bromo-4-fluorobenzo[c][1,2,5]oxadiazole I-26c using a procedure essentially the same as for 1-21. ¹H NMR (400 MHz, DMSO-d6) δ 7.97 (d, J=9.2 Hz, 1H), 7.59 (dd, J=9.2, 6.3 Hz, 1H), 7.30 (d, J=11.7 Hz, 1H), 6.98 (d, J=8.2 Hz, 1H), 5.88 (s, 2H).

### Intermediate 52 (I-52)

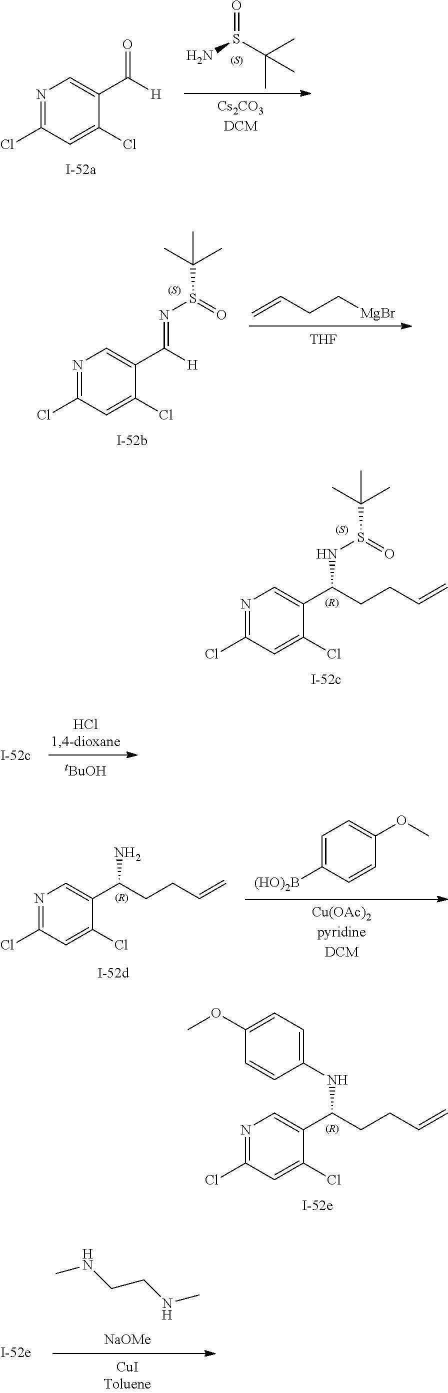

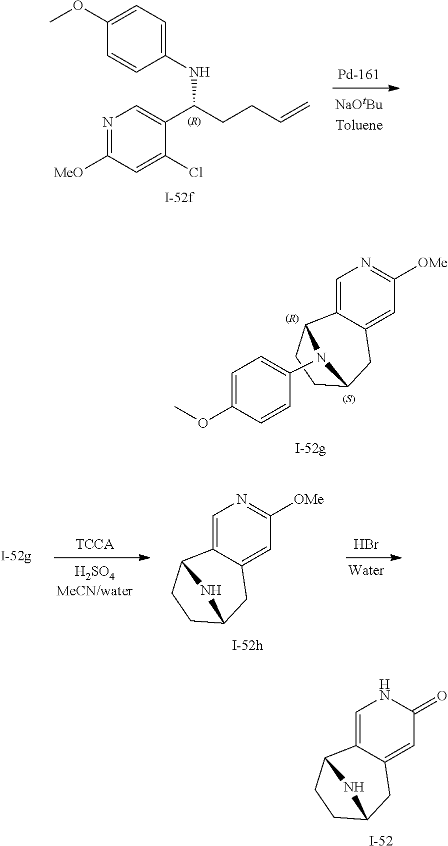

Step 1: To a solution of 4,6-dichloronicotinaldehyde I-52a (17.3 g, 88.5 mmol) and(S)-2-methylpropane-2-sulfinamide (10.7 g, 88.5 mmol) in DCM (150 ml) was added cesium carbonate (28.8 g, 88.5 mmol). The resultant mixture was stirred at RT for 16 h. The material was filtered and the solid residue was washed with DCM (150 ml). The solvent was evaporated to give(S)—N-((4,6-dichloropyridin-3-yl)methylene)-2-methylpropane-2-sulfinamide I-52b as an off white solid. ¹H NMR (400 MHz, DMSO-d6) δ 8.97 (s, 1H), 8.76 (s, 1H), 8.04 (s, 1H), 1.21 (s, 9H).
Step 2: A solution of but-3-en-1-ylmagnesium bromide (0.5 M in THF) (60.9 ml, 30.4 mmol) was slowly added to a solution of(S)—N-((4,6-dichloropyridin-3-yl)methylene)-2-methylpropane-2-sulfinamide I-52b (5.00 g, 17.9 mmol) in THF (100 mL) at −78° C. The reaction mixture was allowed to slowly warm up to RT over 16 h. The reaction was cooled to 0-10° C. and saturated aqueous NH₄Cl (100 ml) was added and stirred for 5 min. The layers were separated and the aqueous phase was extracted with EtOAc (2×250 ml). The combined organics were washed with brine (50 ml), dried with Na₂SO₄ and concentrated in vacuo. The product was purified by chromatography on silica gel (0-30% MTBE in DCM) to afford(S)—N—((R)-1-(4,6-dichloropyridin-3-yl) pent-4-en-1-yl)-2-methylpropane-2-sulfinamide I-52c as a clear yellow oil. ¹H NMR (400 MHz, DMSO-d6) δ 8.53 (s, 1H), 7.77 (s, 1H), 5.85-5.75 (m, 2H), 5.07 (dq, J=17.2, 1.6 Hz, 1H), 5.00 (ddt, J=10.3, 2.3, 1.3 Hz, 1H), 4.66-4.56 (m, 1H), 2.20-2.05 (m, 2H), 1.98 (dtd, J=13.9, 8.0, 5.7 Hz, 1H), 1.83 (ddt, J=13.3, 8.7, 6.5 Hz, 1H), 1.07 (s, 9H).
Step 3: A solution of HCl (4 M in dioxane) (41 ml, 0.16 mol) was added to a solution of(S)—N—((R)-1-(4,6-dichloropyridin-3-yl) pent-4-en-1-yl)-2-methylpropane-2-sulfinamide 1-52c (11 g, 33 mmol) in ^{t}BuOH (50 ml) and the reaction mixture was stirred at RT for 90 min. The reaction mixture was cooled in an ice bath and water (220 ml) was added and stirred for 10 min. The aqueous was extracted with MTBE (3×30 ml). The organic layer was extracted with water (2×30 ml). The aqueous was basified using saturated aqueous NaHCO₃ solution and more solid NaHCO₃ and the mixture was stirred for 15 min. The product was extracted with MTBE (3×100 ml). The combined oragnics were washed with water (500 ml), dried with Na₂SO₄ and concentrated in vacuo to to give (R)-1-(4,6-dichloropyridin-3-yl) pent-4-en-1-amine 1-52d as an orange oil. ¹H NMR (400 MHz, DMSO-d6) δ 8.60 (s, 1H), 7.69 (s, 1H), 5.80 (ddt, J=16.9, 10.1, 6.6 Hz, 1H), 5.01 (dq, J=17.2, 1.7 Hz, 1H), 4.94 (ddt, J=10.2, 2.3, 1.3 Hz, 1H), 4.15 (dd, J=8.0, 5.2 Hz, 1H), 2.17-2.01 (m, 4H), 1.74-1.54 (m, 2H).
Step 4: To a solution of (R)-1-(4,6-dichloropyridin-3-yl) pent-4-en-1-amine I-52d (7.04 g, 30.5 mmol) in DCM (70 ml) was added (4-methoxyphenyl) boronic acid (13.9 g, 91.4 mmol), copper (II) acetate (6.09 g, 33.5 mmol) and Et₃N (21.2 ml, 152 mmol). The resultant mixture was stirred at RT for 20 h. A further portion of copper (II) acetate (2.21 g, 12.2 mmol), (4-methoxyphenyl) boronic acid (5.55 g, 36.6 mmol) and Et₃N (5.09 ml, 36.6 mmol) was added and the mixture was stirred for a further 20 h. 1 M HCl (200 ml) was added and the layers were separated. Ammonium hydroxide solution (28% w/v, 100 and 200 ml) was added to the organic and aqueous layers respectively. The layers were separated and the aqueous was extracted with DCM (100 ml). The combined organics were washed with water (100 ml) and dried with MgSO₄. The product was purified by chromatography on silica gel (0-50% EtOAc/isohexane) to afford (R)—N-(1-(4,6-dichloropyridin-3-yl) pent-4-en-1-yl)-4-methoxyaniline 1-52e as a thick colourless oil. ¹H NMR (400 MHz, DMSO-d6) δ 8.42 (s, 1H), 7.75 (s, 1H), 6.70-6.59 (m, 2H), 6.46-6.34 (m, 2H), 6.01 (d, J=8.5 Hz, 1H), 5.90-5.77 (m, 1H), 5.08-4.94 (m, 2H), 4.64 (td, J=8.6, 4.9 Hz, 1H), 3.58 (s, 3H), 2.27 (ddd, J=12.7, 9.3, 6.1 Hz, 1H), 2.22-2.08 (m, 1H), 1.92-1.72 (m, 2H).
Step 5: To a solution of (R)—N-(1-(4,6-dichloropyridin-3-yl) pent-4-en-1-yl)-4-methoxyaniline 1-52e (2.73 g, 8.10 mmol) in toluene (20 ml) was added N,N-dimethylethane-1,2-diamine (86.8 μl, 810 μmol), copper (I) iodide (30.8 mg, 162 μmol) and sodium methoxide (656 mg, 243 μmol). The resultant mixture was heated at 100° C. for 96 h. The reaction mixture was filtered through a pad of celite and the filtrate was concentrated in vacuo. The product was purified by chromatography on silica gel (0-30% MTBE in isohexane) to afford (R)—N-(1-(4-chloro-6-methoxypyridin-3-yl) pent-4-en-1-yl)-4-methoxyaniline I-52f as a thick yellow oil. ¹H NMR (400 MHz, DMSO-d6) δ 8.17 (s, 1H), 6.94 (s, 1H), 6.66-6.62 (m, 2H), 6.43-6.37 (m, 2H), 5.91 (d, J=8.5 Hz, 1H), 5.89-5.78 (m, 1H), 5.07-4.93 (m, 2H), 4.57 (td, J=8.5, 5.1 Hz, 1H), 3.79 (s, 3H), 3.58 (s, 3H), 2.27 (dt, J=14.3, 7.3 Hz, 1H), 2.22-2.07 (m, 1H), 1.80 (dddd, J=20.8, 13.8, 10.1, 5.1 Hz, 2H).
Step 6: Pd-161 (763.6 mg, 1.65 mmol) and NaOtBu (2.38 g, 24.8 mmol) were placed in a 3-necked RB flask, which was purged under vacuum and backfilled with N₂ (3 times). (R)—N-(1-(4-chloro-6-methoxypyridin-3-yl) pent-4-en-1-yl)-4-methoxyaniline I-52f (5.79 g, 16.52 mmol) was purged under vacuum and backfilled with N₂ (3 times). Toluene (180 ml) was added to amine and the resultant solution was transferred to the 3-necked RB flask. The RB flask was purged under vacuum and backfilled with N₂ (3 times). The resultant mixture was heated at 95° C. for 2 h. The reaction was cooled and filtered through a pad of celite. The filter cake was washed with EtOAc. The filtrate was concentrated in vacuo. The product was purified by chromatography on silica gel (0-50% EtOAc/isohexane) to afford (6S,9R)-3-methoxy-10-(4-methoxyphenyl)-6,7,8,9-tetrahydro-5H-6,9-epiminocyclohepta[c]pyridine I-52g as a light orange solid. ¹H NMR (500 MHz, DMSO-d6) δ 8.01 (s, 1H), 6.82-6.73 (m, 2H), 6.73-6.64 (m, 2H), 6.39 (s, 1H), 4.83 (d, J=5.5 Hz, 1H), 4.43 (m, 1H), 3.74 (s, 3H), 3.61 (s, 3H), 3.04 (dd, J=18.0, 4.9 Hz, 1H), 2.41 (d, J=17.9 Hz, 1H), 2.32-2.14 (m, 2H), 1.84-1.63 (m, 2H).
Step 7: To a solution of (6S,9R)-3-methoxy-10-(4-methoxyphenyl)-6,7,8,9-tetrahydro-5H-6,9-epiminocyclohepta[c]pyridine I-52g (2.00 g, 6.61 mmol) in MeCN (75 ml) and water (75 ml) was added sulfuric acid (6.6 ml, 1 M, 6.61 mmol) followed by trichloroisocyanuric acid (769 mg, 3.31 mmol). The reaction mixture was stirred at RT for 16 h. The mixture was extracted with DCM (3×200 ml). The combined organics were extracted with water (50 ml). The aqueous layer was basified with KOH (3.6 ml, 5 M) and extracted with 10% MeOH in DCM (300 ml). More KOH (1.8 ml, 5 M) was added and the aqueous layer was extracted with 10% MeOH in DCM (100 ml). A further portion of KOH (1.8 ml, 5 M) was added and the aqueous layer was extracted with 10% MeOH in DCM (150 ml). The combined organics were dried over Na₂SO₄ and concentrated in vacuo to give

(6S,9R)-3-methoxy-6,7,8,9-tetrahydro-5H-6,9-epiminocyclohepta[c]pyridine I-52 h as a brown oil. ¹H NMR (400 MHz, DMSO-d6) δ 7.78 (s, 1H), 6.47 (s, 1H), 4.17-4.11 (m, 1H), 3.76 (s, 3H), 3.66 (td, J=5.2, 2.7 Hz, 1H), 2.95 (ddd, J=17.4, 3.9, 2.4 Hz, 1H), 2.57 (s, 1H), 2.46 (dt, J=17.3, 1.2 Hz, 1H), 1.96-1.81 (m, 2H), 1.72-1.60 (m, 1H), 1.50-1.36 (m, 1H).
Step 8: A solution of (6S,9R)-3-methoxy-6,7,8,9-tetrahydro-5H-6,9-epiminocyclohepta[c]pyridine I-52h (0.99 g, 5.2 mmol) in HBr (48% in water) (8.8 ml, 78 mmol) was heated at reflux for 16 h. The mixture was concentrated in vacuo and the concentrate was diluted with MeOH, loaded onto a SCX cartridge (80 g), the cartridge was washed with MeOH and the product was eluted with NH₃ in MeOH solution (0.7M). To give (6S,9R)-2,5,6,7,8,9-hexahydro-3H-6,9-epiminocyclohepta[c]pyridin-3-one I-52 as a brown solid. ¹H NMR (400 MHz, DMSO-d6) δ 11.11 (s, 1H), 7.03 (s, 1H), 6.01 (s, 1H), 4.04 (d, J=5.4 Hz, 1H), 3.62 (t, J=5.9 Hz, 1H), 2.83 (dd, J=17.7, 5.1 Hz, 1H), 2.40 (dt, J=17.7, 1.3 Hz, 1H), 1.92-1.78 (m, 2H), 1.68-1.56 (m, 1H), 1.45 (dt, J=9.3, 4.8 Hz, 1H), Exchangeable NH not observed

The following intermediates could be prepared using appropriate starting materials in an analogous procedure to that used in the preparation of 1a described in Experimental Scheme 1.

| | | Method | |
|---|---|---|---|
| | | LCMS Rt | |
| Int | Structure | [M + H]+ | NMR |
| 2a | 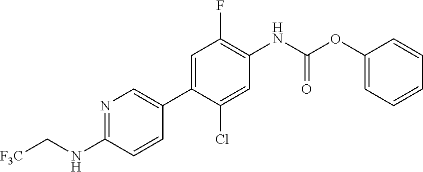 [SMILES: O=C(Nc1cc(Cl)c(-c2ccc(NCC(F)(F)F)nc2)cc1F)Oc1ccccc1] | Method 3 439.9, 441.9 at 1.80 min | ¹H NMR (400 MHz, CDCl₃) δ 8.41 (s, 1H), 8.05 (d, J = 2.3 Hz, 1H), 7.97-7.86 (m, 1H), 7.43 (t, J = 7.8 Hz, 2H), 7.30 (d, J = 7.3 Hz, 1H), 7.24-7.19 (m, 3H), 7.10 (d, J = 10.9 Hz, 1H), 6.88 (d, J = 9.3 Hz, 1H), 4.81 (s, 1H), 4.13-3.97 (m, 2H). |
| | Phenyl (5-chloro-2-fluoro-4-(6- | | |
| | ((2,2,2-trifluoroethyl)amino)- | | |
| | pyridin-3-yl)phenyl)carbamate | | |
| 3a | 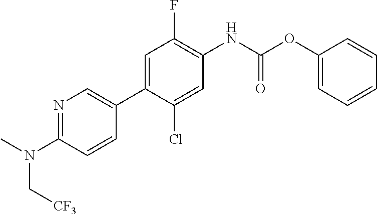 [SMILES: CN(CC(F)(F)F)c1ccc(-c2cc(F)c(NC(=O)Oc3ccccc3)cc2Cl)cn1] | Method 3 453.9, 455.9 at 2.00 min | ¹H NMR (400 MHz, CDCl₃) δ 8.32 (d, J = 7.3 Hz, 1H), 8.24 (d, J = 2.4 Hz, 1H), 7.69 (d, J = 8.7 Hz, 1H), 7.46-7.39 (m, 2H), 7.31-7.27 (m, 1H), 7.24-7.17 (m, 3H), 7.11 (d, J = 11.3 Hz, 1H), 6.68 (d, J = 8.8 Hz, 1H), 4.38 (q, J = 9.0 Hz, 2H), 3.20 (s, 3H). |
| | phenyl (5-chloro-2-fluoro-4-(6- | | |
| | (methyl(2,2,2-trifluoroethyl)- | | |
| | amino)pyridin-3- | | |
| | yl)phenyl)carbamate | | |
| 4a | 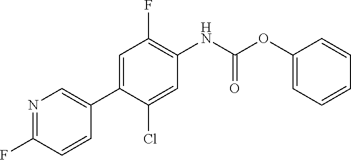 [SMILES: O=C(Nc1cc(Cl)c(-c2ccc(F)nc2)cc1F)Oc1ccccc1] | Method 3 360.7 at 1.81 min | ¹H NMR (400 MHz, CDCl₃) δ 8.39 (d, J = 7.4 Hz, 1H), 8.26 (d, J = 2.5 Hz, 1H), 7.90 (td, J = 8.0, 2.5 Hz, 1H), 7.43 (t, J = 7.8 Hz, 2H), 7.33- 7.26 (m, 2H), 7.24-7.19 (m, 2H), 7.13 (d, J = 11.0 Hz, 1H), 7.02 (dd, J = 8.5, 3.0 Hz, 1H). |
| | Phenyl (5-chloro-2-fluoro-4-(6- | | |
| | fluoropyridin-3-yl)phenyl)- | | |
| | carbamate | | |
| 5a | 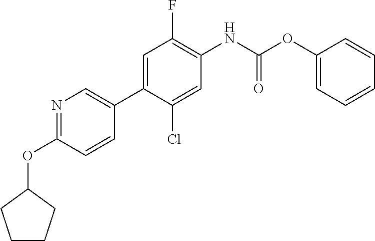 [SMILES: O=C(Nc1cc(Cl)c(-c2ccc(OC3CCCC3)nc2)cc1F)Oc1ccccc1] | Method 3 427.0, 429.0 at 2.17 min | ¹H NMR (400 MHz, CDCl₃) δ 8.36 (d, J = 7.4 Hz, 1H), 8.26 (s, 1H), 7.83 (d, J = 8.6 Hz, 1H), 7.47-7.38 (m, 2H), 7.34-7.27 (m, 1H), 7.24- 7.19 (m, 2H), 7.12 (d, J = 11.1 Hz, 1H), 6.86 (d, J = 8.7 Hz, 1H), 5.53 (s, 1H), 2.13-2.00 (m, 2H), 1.95- 1.77 (m, 5H), 1.68 (dd, J = 9.5, 5.6 Hz, 2H). |
| | Phenyl (5-chloro-4-(6- | | |
| | (cyclopentyloxy)pyridin-3-yl)-2- | | |
| | fluorophenyl)carbamate | | |
| 6a | 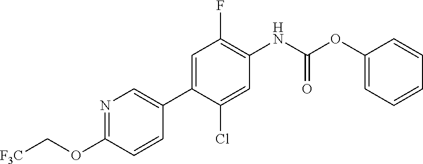 [SMILES: O=C(Nc1cc(Cl)c(-c2ccc(OCC(F)(F)F)nc2)cc1F)Oc1ccccc1] | Method 3 458.9, 460.9 at 2.07 min | ¹H NMR (400 MHz, CDCl₃) δ 8.38 (s, 1H), 7.96 (s, 1H), 7.56 (d, J = 10.4 Hz, 1H), 7.43 (t, J = 7.7 Hz, 2H), 7.35-7.18 (m, 4H), 7.12 (d, J = 10.9 Hz, 1H), 4.88 (q, J = 8.5 Hz, 2H) |
| | Phenyl (5-chloro-2-fluoro-4-(5- | | |
| | fluoro-6-(2,2,2-trifluoroethoxy)- | | |
| | pyridin-3-yl)phenyl)carbamate | | |
| 7a | 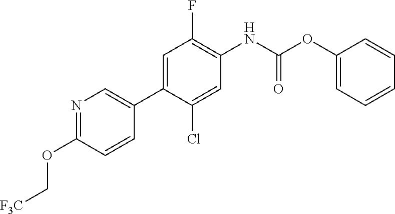 [SMILES: O=C(Nc1cc(Cl)c(-c2ccc(OCC(F)(F)F)nc2)cc1F)Oc1ccccc1] | Method 3 441.9, 443.9 at 1.86 min | ¹H NMR (400 MHz, CDCl₃) δ 8.41 (d, J = 7.2 Hz, 1H), 7.96 (d, J = 9.2 Hz, 1H), 7.60 (d, J = 11.3 Hz, 1H), 7.43 (tt, J = 7.7, 2.2 Hz, 2H), 7.32- 7.26 (m, 2H), 7.24-7.19 (m, 3H), 5.01 (q, J = 8.3 Hz, 2H). |
| | Phenyl (5-chloro-2-fluoro-4-(6- | | |
| | (2,2,2-trifluoroethoxy)pyridazin- | | |
| | 3-yl)phenyl)carbamate | | |
| 8a | 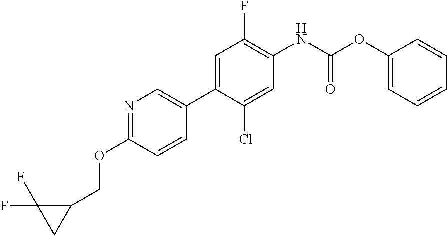 [SMILES: O=C(Nc1cc(Cl)c(-c2ccc(OCC3CC3(F)F)nc2)cc1F)Oc1ccccc1] | Method 3 448.9, 450.9 at 2.03 min | ¹H NMR (400 MHz, CDCl₃) δ 8.46- 8.27 (m, 1H), 8.18 (s, 1H), 7.71 (d, J = 8.5 Hz, 1H), 7.43 (t, J = 7.7 Hz, 2H), 7.29 (d, J = 7.5 Hz, 1H), 7.21 (s, 3H), 7.11 (d, J = 11.1 Hz, 1H), 6.84 (d, J = 8.6 Hz, 1H), 4.67-4.26 (m, 2H), 2.17 (s, 1H), 1.30 (d, J = 12.1 Hz, 1H). (1 exchangeable proton not observed) |
| | Phenyl (5-chloro-4-(6-((2,2- | | |
| | difluorocyclopropyl)methoxy)- | | |
| | pyridin-3-yl)-2-fluorophenyl)- | | |
| | carbamate | | |
| 10a | 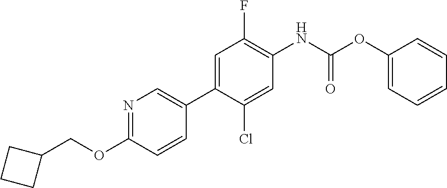 [SMILES: O=C(Nc1cc(Cl)c(-c2ccc(OCC3CCC3)nc2)cc1F)Oc1ccccc1] | Method 3 426.7 at 2.21 min | ¹H NMR (400 MHz, CDCl₃) δ 8.34 (d, J = 7.4 Hz, 1H), 8.20 (d, J = 2.5 Hz, 1H), 7.71 (dd, J = 8.7, 2.5 Hz, 1H), 7.43 (t, J = 7.8 Hz, 2H), 7.29 (d, J = 7.3 Hz, 1H), 7.24-7.19 (m, 3H), 7.11 (d, J = 11.2 Hz, 1H), 6.83 (d, J = 8.6 Hz, 1H), 4.34 (d, J = 6.8 Hz, 2H), 2.81 (p, J = 7.5 Hz, 1H), 2.16 (tt, J = 9.5, 5.2 Hz, 2H), 1.93 (ddt, J = 22.8, 17.6, 8.5 Hz, 4H). |
| | Phenyl (5-chloro-4-(6- | | |
| | (cyclobutylmethoxy)pyridin-3-yl)- | | |
| | 2-fluorophenyl)carbamate | | |
| 12a | 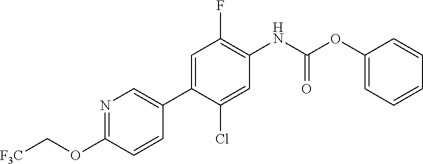 [SMILES: O=C(Nc1cc(Cl)c(-c2ccc(OCC(F)(F)F)nc2)cc1F)Oc1ccccc1] | Method 2 441.2, 443.1 at 2.38 min | ¹H NMR (400 MHz, DMSO-d6) δ 10.33 (s, 1H), 8.30 (dd, J = 2.5, 0.8 Hz, 1H), 8.02-7.91 (m, 2H), 7.51 (d, J = 11.1 Hz, 1H), 7.48-7.40 (m, 2H), 7.32-7.22 (m, 3H), 7.10 (dd, J = 8.5, 0.7 Hz, 1H), 5.06 (q, J = 9.1 Hz, 2H). |
| | Phenyl (5-chloro-2-fluoro-4-(6- | | |
| | (2,2,2-trifluoroethoxy)pyridin-3- | | |
| | yl)phenyl) carbamate | | |
| 13a | 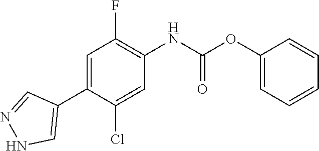 [SMILES: O=C(Nc1cc(Cl)c(-c2cn[nH]c2)cc1F)Oc1ccccc1] | Method 3 331.9, 333.9 at 1.65 min | |
| | Phenyl (5-chloro-2-fluoro-4-(1H- | | |
| | pyrazol-4-yl)phenyl)carbamate | | |
| 14a | 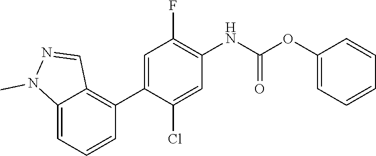 [SMILES: Cn1ncc2c(-c3cc(F)c(NC(=O)Oc4ccccc4)cc3Cl)cccc21] | Method 3 395.9, 397.9 at 1.85 min | ¹H NMR (400 MHz, CDCl₃) δ 8.42 (d, J = 6.9 Hz, 1H), 7.91 (s, 1H), 7.55 (t, J = 7.7 Hz, 1H), 7.46 (d, J = 5.8 Hz, 1H), 7.43 (d, J = 7.8 Hz, 2H), 7.30 (d, J = 7.5 Hz, 2H), 7.25- 7.19 (m, 4H), 4.21 (s, 3H). |
| | Phenyl (5-chloro-2-fluoro-4-(1- | | |
| | methyl-1H-indazol-4- | | |
| | yl)phenyl) carbamate | | |
| 15a | 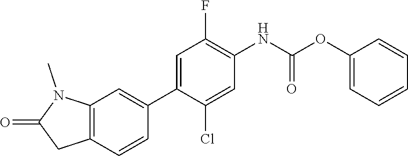 [SMILES: CN1C(=O)Cc2ccc(-c3cc(F)c(NC(=O)Oc4ccccc4)cc3Cl)cc21] | Method 3 410.7 at 1.78 min | ¹H NMR (400 MHz, CDCl₃) δ 8.34 (d, J = 7.3 Hz, 1H), 7.43 (t, J = 7.8 Hz, 2H), 7.30 (dd, J = 7.8, 4.1 Hz, 2H), 7.25-7.19 (m, 3H), 7.15 (d, J = 11.2 Hz, 1H), 7.07 (dt, J = 7.5, 1.1 Hz, 1H), 6.87 (s, 1H), 3.58 (s, 2H), 3.24 (d, J = 0.8 Hz, 3H). |
| | Phenyl (5-chloro-2-fluoro-4-(1- | | |
| | methyl-2-oxoindolin-6-yl)phenyl)- | | |
| | carbamate | | |
| 16a | 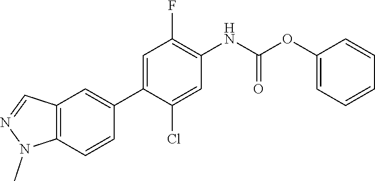 [SMILES: Cn1ncc2cc(-c3cc(F)c(NC(=O)Oc4ccccc4)cc3Cl)ccc21] | Method 3 395.7 at 1.89 min | ¹H NMR (400 MHz, CDCl₃) δ 8.34 (d, J = 7.3 Hz, 1H), 8.04 (s, 1H), 7.76 (d, J = 1.3 Hz, 1H), 7.51-7.39 (m, 4H), 7.32-7.27 (m, 1H), 7.25- 7.20 (m, 3H), 7.18 (d, J = 11.3 Hz, 1H), 4.12 (s, 3H). |
| | Phenyl (5-chloro-2-fluoro-4-(1- | | |
| | methyl-1H-indazol-5- | | |
| | yl)phenyl) carbamate | | |
| 17a | 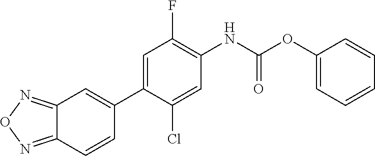 [SMILES: O=C(Nc1cc(Cl)c(-c2ccc3nonc3c2)cc1F)Oc1ccccc1] | Method 3 384.9, 386.9 at 1.81 min | ¹H NMR (400 MHz, CDCl₃) δ 9.03 (s, 1H), 8.50 (d, J = 7.2 Hz, 1H), 8.28-8.14 (m, 1H), 7.44 (t, J = 7.8 Hz, 2H), 7.39-7.25 (m, 3H), 7.22 (s, 1H). 1 exchangeable proton not observed. |
| | Phenyl (4-([1,2,5]oxadiazolo[3,4- | | |
| | b]pyridin-6-yl)-5-chloro-2- | | |
| | fluorophenyl) carbamate | | |
| 18a | 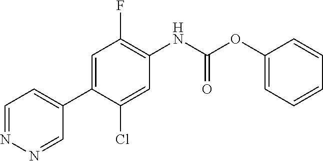 [SMILES: O=C(Nc1cc(Cl)c(-c2ccnnc2)cc1F)Oc1ccccc1] | Method 3 343.8 at 1.51 min | ¹H NMR (400 MHz, CDCl₃) δ 9.37 (s, 1H), 9.34 (d, J = 5.4 Hz, 1H), 8.49 (d, J = 7.2 Hz, 1H), 7.75 (s, 1H), 7.44 (t, J = 7.8 Hz, 2H), 7.34 (s, 1H), 7.30 (t, J = 7.4 Hz, 1H), 7.25-7.19 (m, 3H). |
| | Phenyl (5-chloro-2-fluoro-4- | | |
| | (pyridazin-4-yl)phenyl)carbamate | | |
| 19a | 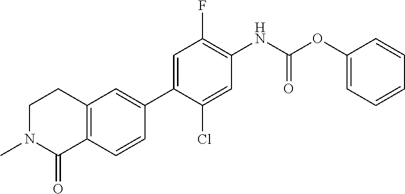 [SMILES: CN1CCc2cc(-c3cc(F)c(NC(=O)Oc4ccccc4)cc3Cl)ccc2C1=O] | Method 3 424.7 at 1.78 min | ¹H NMR (400 MHz, CDCl₃) δ 8.34 (d, J = 7.3 Hz, 1H), 8.14 (d, J = 8.0 Hz, 1H), 7.47-7.34 (m, 3H), 7.31- 7.27 (m, 1H), 7.25-7.19 (m, 4H), 7.13 (d, J = 11.2 Hz, 1H), 3.61 (t, J = 6.6 Hz, 2H), 3.18 (s, 3H), 3.06 (t, J = 6.6 Hz, 2H). |
| | Phenyl (5-chloro-2-fluoro-4-(2- | | |
| | methyl-1-oxo-1,2,3,4- | | |
| | tetrahydroisoquinolin-6- | | |
| | yl)phenyl)carbamate | | |
| 20a | 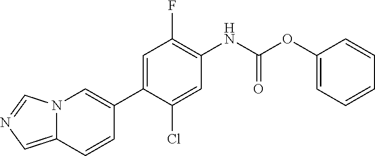 [SMILES: O=C(Nc1cc(Cl)c(-c2ccc3cncn3c2)cc1F)Oc1ccccc1] | Method 3 381.9, 383.9 at 1.38 min | ¹H NMR (400 MHz, CDCl₃) δ 8.38 (d, J = 7.4 Hz, 1H), 8.24 (s, 1H), 8.01 (d, J = 1.5 Hz, 1H), 7.50 (d, J = 8.8 Hz, 2H), 7.47-7.38 (m, 3H), 7.31-7.26 (m, 1H), 7.24-7.20 (m, 2H), 7.18 (d, J = 11.0 Hz, 1H), 6.82 (dd, J = 9.6, 1.4 Hz, 1H). |
| | Phenyl (5-chloro-2-fluoro-4- | | |
| | (imidazo[1,5-a]pyridin-6- | | |
| | yl)phenyl)carbamate | | |
| 21a | 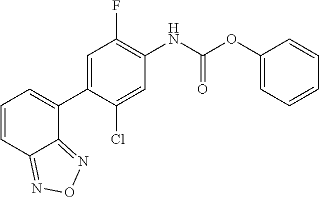 [SMILES: O=C(Nc1cc(Cl)c(-c2cccc3nonc23)cc1F)Oc1ccccc1] | Method 3 383.9, 385.9 at 1.90 min | ¹H NMR (400 MHz, CDCl₃) δ 8.45 (d, J = 7.4 Hz, 1H), 7.94-7.85 (m, 1H), 7.56-7.50 (m, 2H), 7.47- 7.37 (m, 3H), 7.32-7.28 (m, 2H), 7.25-7.21 (m, 2H). |
| | Phenyl (4-(benzo[c][1,2,5]- | | |
| | oxadiazol-4-yl)-5-chloro-2- | | |
| | fluorophenyl)carbamate | | |
| 22a | 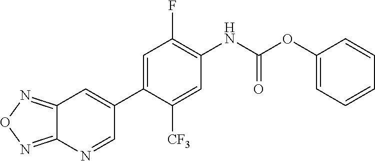 [SMILES: O=C(Nc1cc(C(F)(F)F)c(-c2cnc3nonc3c2)cc1F)Oc1ccccc1] | Method 3 418.9, 419.9 at 1.84 min | ¹H NMR (400 MHz, CDCl₃) δ 8.88 (s, 1H), 8.81 (d, J = 7.4 Hz, 1H), 8.17 (s, 1H), 7.45 (t, J = 7.5 Hz, 3H), 7.31 (t, J = 7.5 Hz, 1H), 7.27- 7.18 (m, 2H). (1 exchangeable proton not observed) |
| | Phenyl (4-([1,2,5]oxadiazolo[3,4- | | |
| | b]pyridin-6-yl)-2-fluoro-5- | | |
| | (trifluoromethyl)phenyl)carbamate | | |
| 23a | 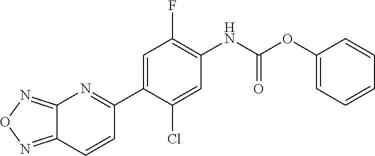 [SMILES: O=C(Nc1cc(Cl)c(-c2ccc3nonc3n2)cc1F)Oc1ccccc1] | Method 3 384.7 at 1.83 min | ¹H NMR (400 MHz, CDCl₃) δ 8.46 (d, J = 7.2 Hz, 1H), 8.27 (d, J = 9.3 Hz, 1H), 7.91 (d, J = 9.3 Hz, 1H), 7.66 (d, J = 11.1 Hz, 1H), 7.44 (t, J = 7.8 Hz, 2H), 7.37 (s, 1H), 7.30 (t, J = 7.4 Hz, 1H), 7.25-7.20 (m, 2H). |
| | Phenyl (4-([1,2,5]oxadiazolo[3,4- | | |
| | b]pyridin-5-yl)-5-chloro-2- | | |
| | fluorophenyl)carbamate | | |
| 24a | 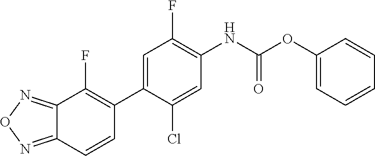 [SMILES: O=C(Nc1cc(Cl)c(-c2ccc3nonc3c2F)cc1F)Oc1ccccc1] | Method 3 401.7 at 1.97 min | ¹H NMR (400 MHz, CDCl₃) δ 8.46 (d, J = 7.3 Hz, 1H), 7.72 (d, J = 9.2 Hz, 1H), 7.44 (t, J = 7.8 Hz, 2H), 7.39 (dd, J = 9.2, 5.9 Hz, 1H), 7.33- 7.27 (m, 2H), 7.25-7.20 (m, 3H). |
| | Phenyl (5-chloro-2-fluoro-4-(4- | | |
| | fluorobenzo[c][1,2,5]oxadiazol-5- | | |
| | yl)phenyl)carbamate | | |

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Bohn, T. et al. (2018). Tumor immunoevasion via acidosis-dependent induction of regulatory tumor-associated macrophages. Nature Immunology, 1319-1326.
Damaghi, M. et al. (2013). pH Sensing and Regulation in Cancer. Frontiers in Physiology.
Gaublomme, J. et al. (2015). Single-Cell Genomics Unveils Critical Regulators of Th17 Cell Pathogenicity. Cell, 1400-1412.
Hernandez, J. (2018). GPR65, a critical regulator of Th17 cell pathogenicity, is regulated by the CRTC2/CREB pathway. The Journal of Immunology, 200 (Supplement).
Korn, T. et al. (2009). IL-17 and Th17 Cells. Annual Reviews in Immunology, 485-517.
Wang, J. et al. (2004). TDAG8 is a proton-sensing and psychosine-sensitive G-protein-coupled receptor. Journal of Biological Chemistry, 45626-45633.
Yoshida, N. et al. (2016). ICER is requisite for Th17 differentiation. Nature Communications, 12993.

1. A process for preparing a compound of formula (I), or a pharmaceutically acceptable salt or solvate thereof, 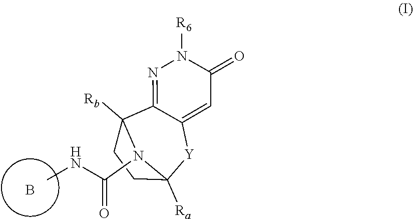
wherein:
ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
Y is CR₁₀R₁₀′, wherein R₁₀ and R₁₀′ are each independently selected from H, F, alkyl, and haloalkyl;
Rₐ and R_{b} are each independently selected from H and alkyl;
R₆ is selected from H, alkyl, cycloalkyl and hydroxyalkyl;
R₁₃, R₁₃′, R₁₄, R₁₄′, R₁₅, R₁₅′, and R₁₆ are each independently selected from H, alkyl, haloalkyl and alkoxyalkyl;
said process comprising the steps of:
(i) treating a compound of formula (IV), where B is defined as above, with a compound of formula (V), where R₂₁ is phenyl optionally substituted with 1 to 5 fluorine atoms, to form a compound of formula (III); and
(ii) treating said compound of formula (III) with a compound of formula (II), or a pharmaceutically acceptable salt thereof, where Y, Rₐ, R_{b} and R₆ are as described above, to form a compound of formula (I);

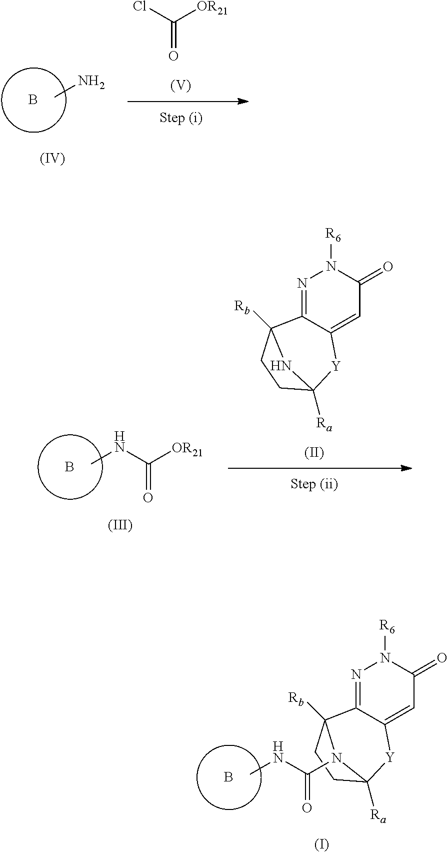

2. A process according to claim 1 wherein Rₐ and R_{b} are both H

3. A process according to any preceding claim, wherein Y is CH₂.

4. A process according to any preceding claim wherein R₆ is selected from H, methyl and hydroxymethyl, and is more preferably H.

5. A process according to any preceding claim, wherein Ring B is: 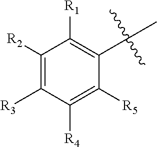
wherein:
R₁, R₄, and R₅ are each independently selected from H, CN, alkyl, alkoxy, haloalkyl, OH, and halo;
R₂ and R₃ are each independently selected from H, OH, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

6. A process according to claim 5, wherein R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl, alkoxy and haloalkoxyl.

7. A process according to claim 5 wherein R₃ is an aryl or heteroaryl group, each of which is optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

8. A process according to claim 5 wherein R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzooxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3(4H)-onyl, more preferably a pyridinyl group; each of which is optionally further substituted by one or more groups independently selected from halo, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, heteroaryl, and O—(CH₂)ₚ-cycloalkyl, where in the latter group, said cycloalkyl group is optionally further substituted by one or more halo, haloalkyl, alkyl or alkoxy groups.

9. A process according to claim 5 wherein R₃ is selected from: 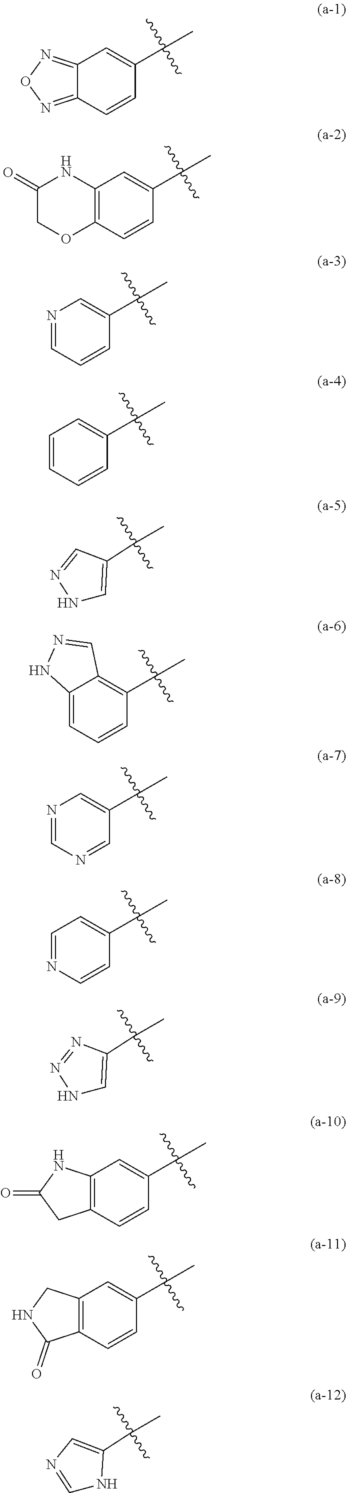 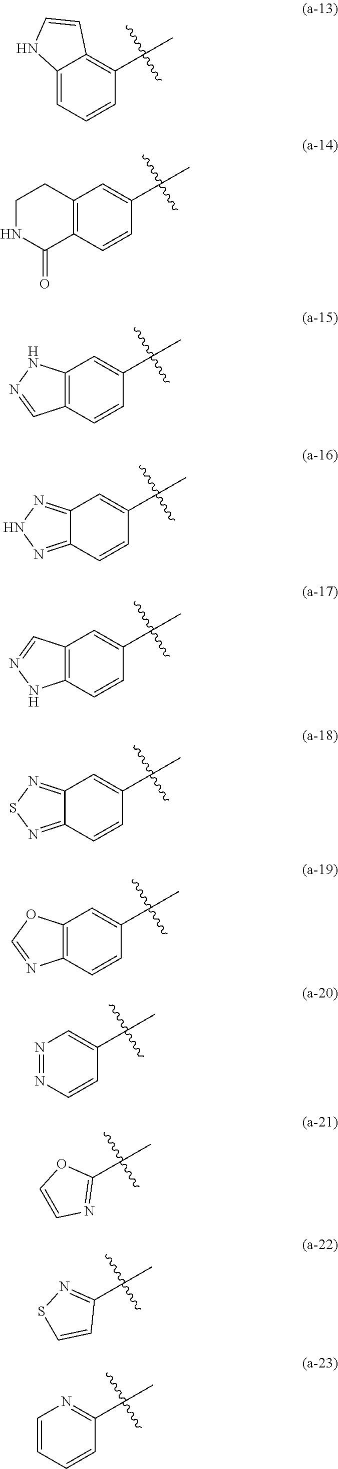 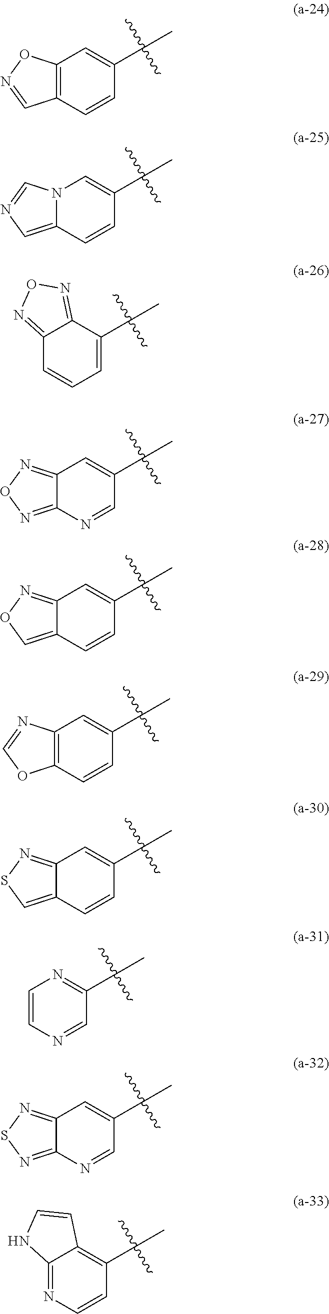 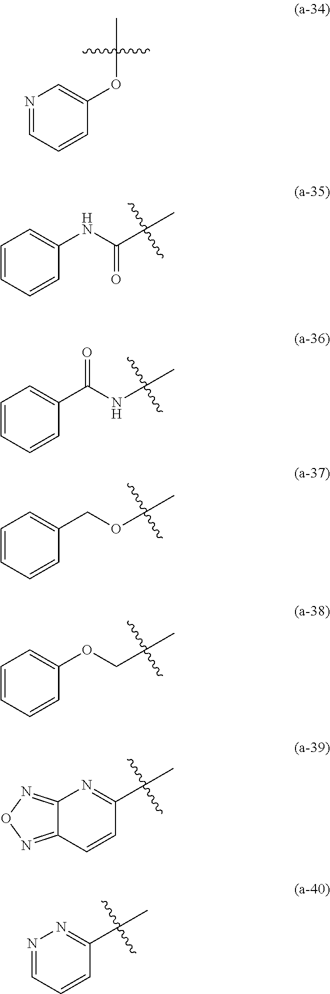
each of which is optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, —(CH₂)_{q}—O-heteroaryl, and O—(CH₂)ₚ-cycloalkyl, where in the latter group, said cycloalkyl group is optionally further substituted by one or more halo, haloalkyl, alkyl or alkoxy groups.

10. A process according to claim 5 wherein R₃ is: 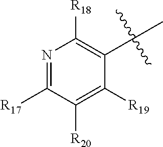
wherein:
R₁₇ is selected from H, alkyl, CN, haloalkyl, NHCO-alkyl, NR₁₃R₁₃′, alkoxy, SO₂-alkyl, halo, O—(CH₂)_{q}-heterocycloalkyl, alkoxy-alkoxy, alkoxy-alkyl, haloalkoxy, alkylamino-alkoxy, dialkylamino-alkoxy, and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆.

11. A process according to claim 10 wherein:
R₁₇ is selected from H, OMe, OEt, OⁱPr, F, SO₂Me, halo, O-cyclobutyl, O-oxetanyl, O—CH₂CF₃, CH₂CF₃, O—CH₂-cyclobutyl, O-cyclopentyl, NHCH₂CF₃, N(Me)CH₂CF₃, O—CH₂-(difluorocyclopropyl), and O—CH₂-cyclopropyl;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

12. A process according to any one of claims 5 to 11, wherein R₂ and R₅ are both H.

13. A process according to any one of claims 5 to 12, wherein R₁ is selected from H, F, Me, MeO, Cl, OH and CN, and is preferably H or F.

14. A process according to any one of claims 5 to 13, wherein R₄ is selected from CI, Br, and CF₃, more preferably Cl.

15. A process according to any one of claims 5 to 14, wherein R₁ is F, R₂ is H, R₄ is Cl and R₅ is H.

16. A process according to any preceding claim wherein the compound of formula (V) is selected from phenyl chloroformate and pentafluorophenyl choroformate.

17. A process according to any preceding claim wherein the compound of formula (V) is phenyl chloroformate.

18. A process according to any preceding claim, wherein step (i) is carried out in the absence of a base.

19. A process according to any preceding claim, wherein step (i) is carried out in an organic solvent, preferably selected from tetrahydrofuran, dichloromethane, dimethylsulfoxide (DMSO), and 2-methyltetrahydrofuran, and mixtures thereof.

20. A process according to any preceding claim, wherein the ratio of compound (V) to compound (IV) in step (i) is about 3:1 to about 1:1, more preferably about 2:1 to about 1:1.

21. A process according to any preceding claim, wherein step (i) is carried out at a temperature of from about 0° C. to about 45° C., preferably from about 10° C. to about 40° C., more preferably from about 20 to about 40° C., more preferably from about 25 to about 35° C.

22. A process according to any preceding claim, wherein step (ii) is carried out in the presence of a base, more preferably a tertiary aliphatic amine base.

23. A process according to claim 22 wherein the base is selected from N,N-diisopropylethylamine (DIEA), triethylamine, tri-ⁿpropylamine, triisopropylamine and tri-ⁿbutylamine, N-methyl morpholine and N-methyl piperidine, more preferably triethylamine.

24. A process according to any preceding claim, wherein step (ii) is carried out in an organic solvent, preferably selected from tetrahydrofuran, dichloromethane, dimethylsulfoxide (DMSO), and 2-methyltetrahydrofuran, and mixtures thereof.

25. A process according to any preceding claim, wherein step (ii) is carried out at a temperature of from about 35° C. to about 65° C., more preferably about 40° C. to about 60° C., more preferably about 40° C. to about 50° C.

26. A process according to any preceding claim, wherein steps (i) and (ii) are carried out without isolating or purifying the compound of formula (III).

27. A process according to any of claims 1-25, which comprises isolating the compound of formula (III) reaction mixture obtained in step (i) prior to carrying out step (ii).

28. A process according to claim 27, wherein the compound of formula (III) is isolated from the reaction mixture and purified prior to carrying out step (ii).

29. A process according to any preceding claim, wherein the compound of formula (II) is in the form of a pharmaceutically acceptable salt, more preferably, a hydrochloride salt.

30. A process according to any preceding claim, wherein the ratio of compound (II), or pharmaceutically acceptable salt thereof, to compound (III) in step (ii) is about 1:1.

31. A process according to any preceding claim, wherein the compound of formula (I) is selected from the following: 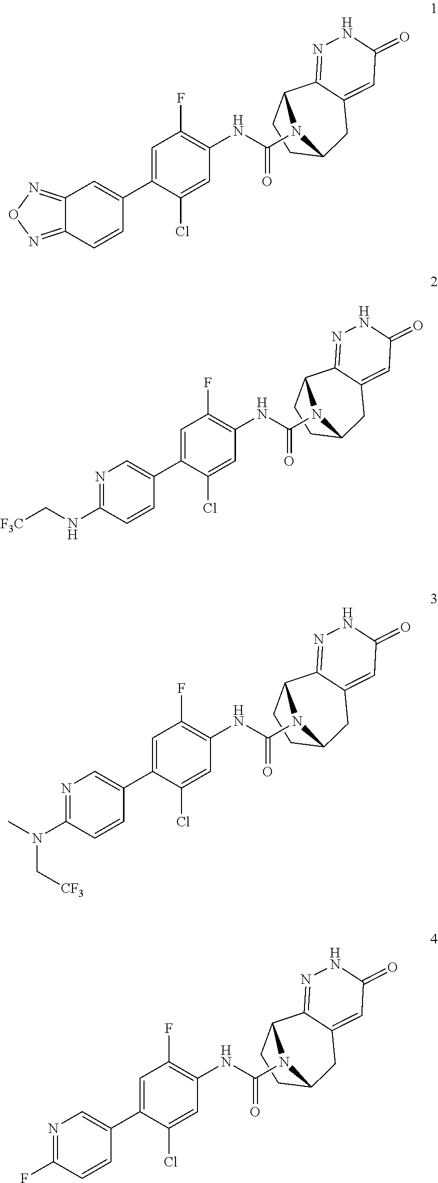 [SMILES: O=C(Nc1cc(Cl)c(-c2ccc3nonc3c2)cc1F)N1[C@H]2CC[C@@H]1c1n[nH]c(=O)cc1C2 ; O=C(Nc1cc(Cl)c(-c2ccc(NCC(F)(F)F)nc2)cc1F)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc(F)nc2)cc1F)N1[C@H]2CC[C@@H]1c1n[nH]c(=O)cc1C2] 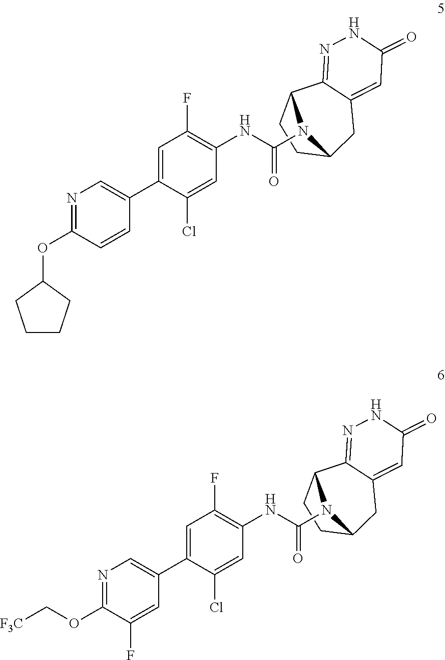 [SMILES: O=C(Nc1cc(Cl)c(-c2ccc(OC3CCCC3)nc2)cc1F)N1[C@H]2CC[C@@H]1c1n[nH]c(=O)cc1C2 ; O=C(Nc1cc(Cl)c(-c2cnc(OCC(F)(F)F)c(F)c2)cc1F)N1[C@H]2CC[C@@H]1c1n[nH]c(=O)cc1C2] 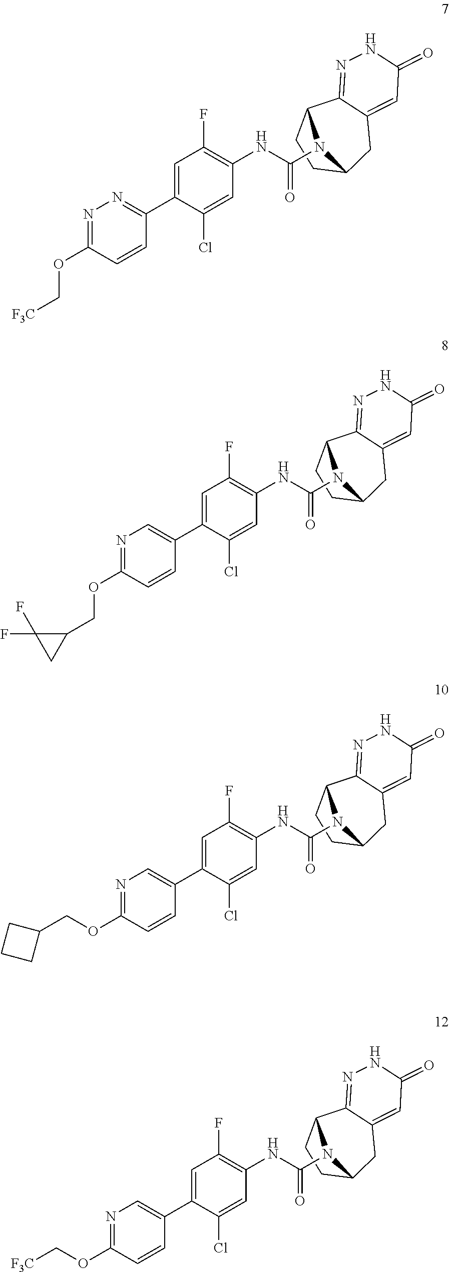 [SMILES: O=C(Nc1cc(Cl)c(-c2ccc(OCC(F)(F)F)nn2)cc1F)N1[C@H]2CC[C@@H]1c1n[nH]c(=O)cc1C2 ; O=C(Nc1cc(Cl)c(-c2ccc(OCC3CC3(F)F)nc2)cc1F)N1[C@H]2CC[C@@H]1c1n[nH]c(=O)cc1C2 ; O=C(Nc1cc(Cl)c(-c2ccc(OCC3CCC3)nc2)cc1F)N1[C@H]2CC[C@@H]1c1n[nH]c(=O)cc1C2 ; O=C(Nc1cc(Cl)c(-c2ccc(OCC(F)(F)F)nc2)cc1F)N1[C@H]2CC[C@@H]1c1n[nH]c(=O)cc1C2] 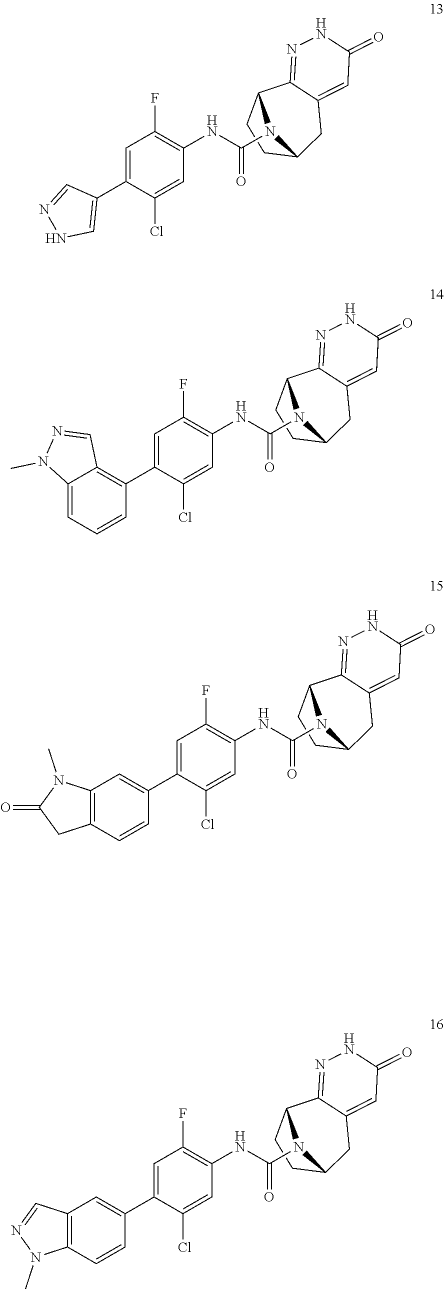 [SMILES: O=C(Nc1cc(Cl)c(-c2cn[nH]c2)cc1F)N1[C@H]2CC[C@@H]1c1n[nH]c(=O)cc1C2 ; Cn1ncc2c(-c3cc(F)c(NC(=O)N4[C@H]5CC[C@@H]4c4n[nH]c(=O)cc4C5)cc3Cl)cccc21 ; CN1C(=O)Cc2ccc(-c3cc(F)c(NC(=O)N4[C@H]5CC[C@@H]4c4n[nH]c(=O)cc4C5)cc3Cl)cc21 ; Cn1ncc2cc(-c3cc(F)c(NC(=O)N4[C@H]5CC[C@@H]4c4n[nH]c(=O)cc4C5)cc3Cl)ccc21] 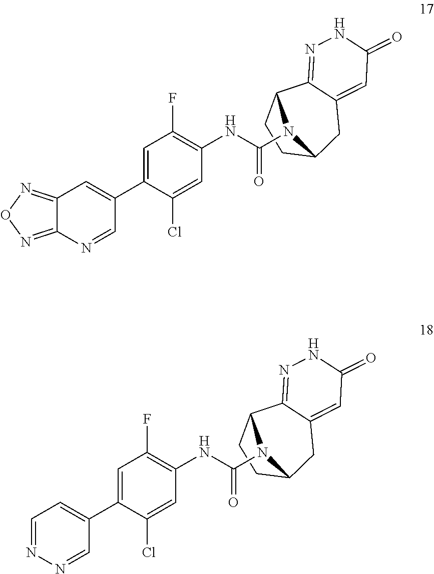 [SMILES: O=C(Nc1cc(Cl)c(-c2cnc3nonc3c2)cc1F)N1[C@H]2CC[C@@H]1c1n[nH]c(=O)cc1C2 ; O=C(Nc1cc(Cl)c(-c2ccnnc2)cc1F)N1[C@H]2CC[C@@H]1c1n[nH]c(=O)cc1C2] 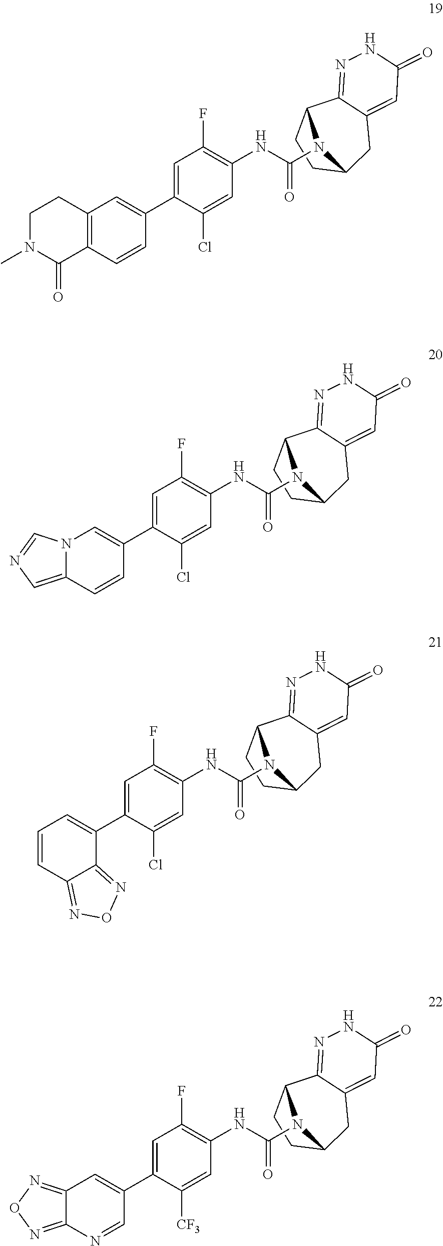 [SMILES: CN1CCc2cc(-c3cc(F)c(NC(=O)N4[C@H]5CC[C@@H]4c4n[nH]c(=O)cc4C5)cc3Cl)ccc2C1=O ; O=C(Nc1cc(Cl)c(-c2ccc3cncn3c2)cc1F)N1[C@H]2CC[C@@H]1c1n[nH]c(=O)cc1C2 ; O=C(Nc1cc(Cl)c(-c2cccc3nonc23)cc1F)N1[C@H]2CC[C@@H]1c1n[nH]c(=O)cc1C2 ; O=C(Nc1cc(C(F)(F)F)c(-c2cnc3nonc3c2)cc1F)N1[C@H]2CC[C@@H]1c1n[nH]c(=O)cc1C2] 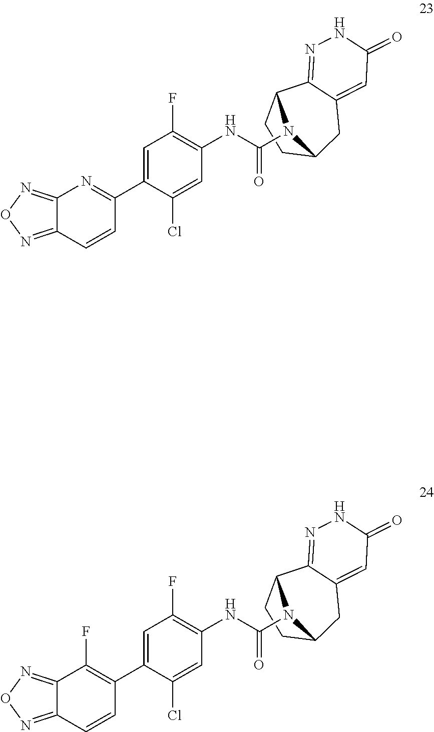 [SMILES: O=C(Nc1cc(Cl)c(-c2ccc3nonc3n2)cc1F)N1[C@H]2CC[C@@H]1c1n[nH]c(=O)cc1C2 ; O=C(Nc1cc(Cl)c(-c2ccc3nonc3c2F)cc1F)N1[C@H]2CC[C@@H]1c1n[nH]c(=O)cc1C2]
and enantiomers thereof, and mixtures of enantiomers thereof, including racemic mixtures, and pharmaceutically acceptable salts and solvates thereof.

32. A compound of formula (III), or a pharmaceutically acceptable salt or solvate thereof: 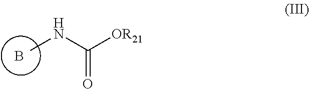
wherein:
ring B is a monocyclic aromatic group which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
R₁₃, R₁₃′, R₁₄, R₁₄′, R₁₅, R₁₅′, and R₁₆ are each independently selected from H, alkyl, haloalkyl and alkoxyalkyl; and
R₂₁ is phenyl optionally substituted with 1 to 5 fluorine atoms.

33. A compound according to claim 32, wherein Ring B is: 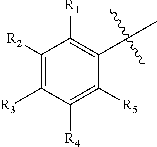
wherein:
R₁, R₄, and R₅ are each independently selected from H, CN, alkyl, alkoxy, haloalkyl, OH, and halo;
R₂ and R₃ are each independently selected from H, OH, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

34. A compound according to claim 33, wherein R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl, alkoxy and haloalkoxyl.

35. A compound according to claim 33, wherein R₃ is an aryl or heteroaryl group, each of which is optionally further substituted by one or more groups independently selected from halo, haloalkyl, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

36. A compound according to claim 33, wherein R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzooxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3 (4H)-onyl, more preferably a pyridinyl group; each of which is optionally further substituted by one or more groups independently selected from halo, alkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, heteroaryl, and O—(CH₂)ₚ-cycloalkyl, where in the latter group, said cycloalkyl group is optionally further substituted by one or more halo, haloalkyl, alkyl or alkoxy groups.

37. A compound according to claim 33 wherein R₃ is selected from: 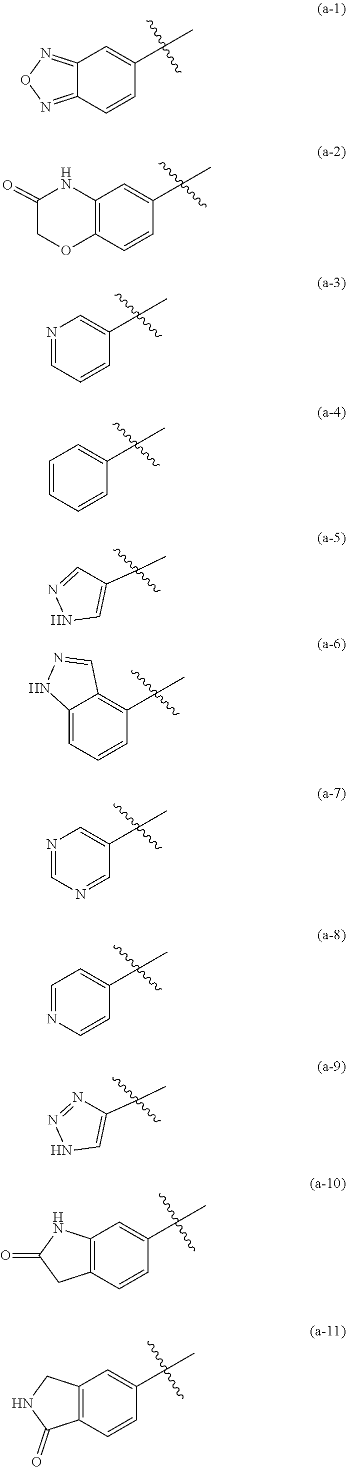 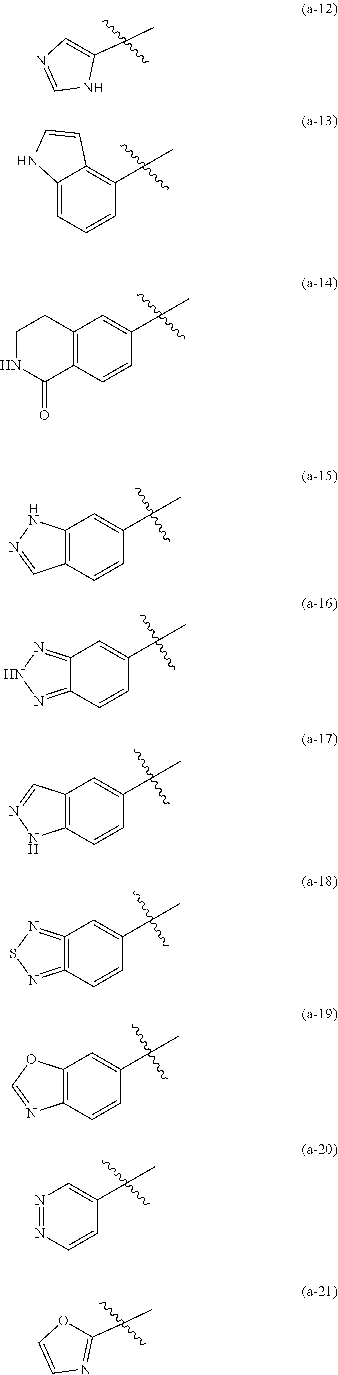 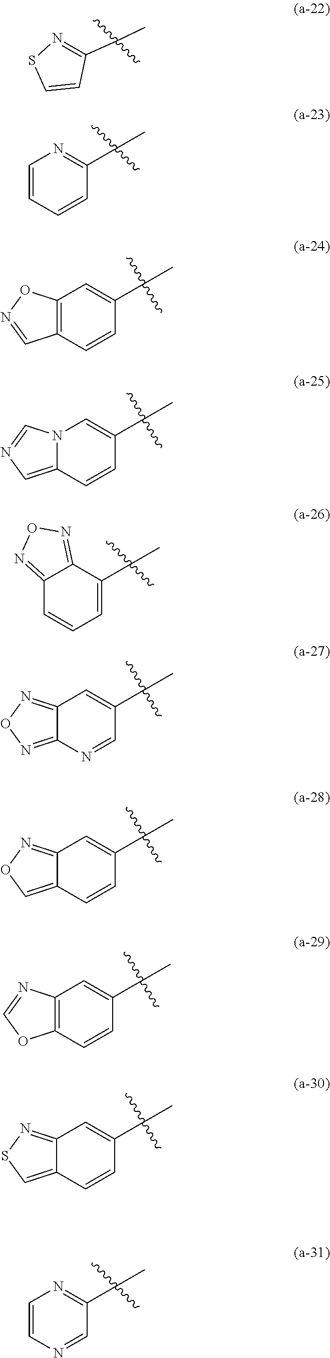 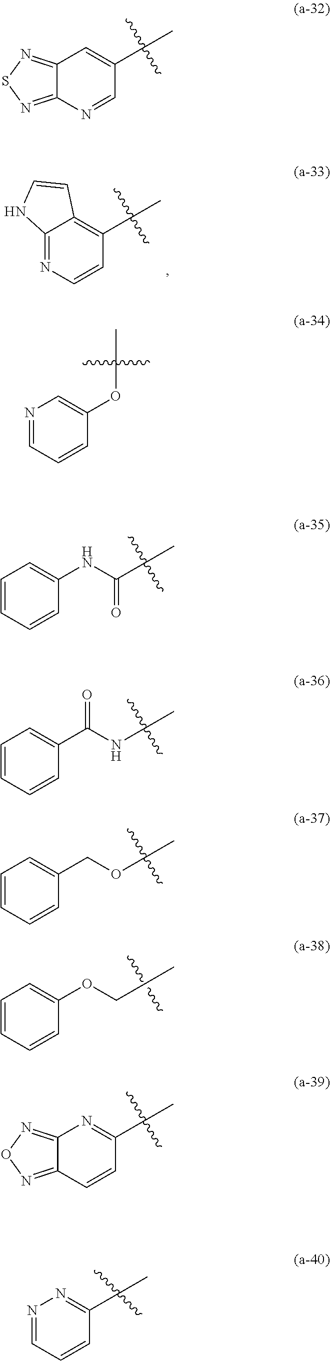
each of which is optionally further substituted by one or more groups independently selected from halo, alkyl, haloalkyl, alkoxy, NHCO-alkyl, NR₁₃R₁₃′, SO₂-alkyl, CN, hydroxyalkyl, CONR₁₄R₁₄′, alkyl-NR₁₅R₁₅′, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, —(CH₂)_{q}—O-heteroaryl, and O—(CH₂)ₚ-cycloalkyl, where in the latter group, said cycloalkyl group is optionally further substituted by one or more halo, haloalkyl, alkyl or alkoxy groups.

38. A compound according to claim 33 wherein R₃ is: 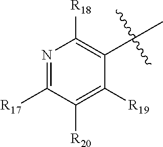
wherein:
R₁₇ is selected from H, alkyl, CN, haloalkyl, NHCO-alkyl, NR₁₃R₁₃′, alkoxy, SO₂-alkyl, halo, O—(CH₂)_{q}-heterocycloalkyl, alkoxy-alkoxy, alkoxy-alkyl, haloalkoxy, alkylamino-alkoxy, dialkylamino-alkoxy, and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆.

39. A compound according to claim 38 wherein:
R₁₇ is selected from H, OMe, OEt, OⁱPr, F, SO₂Me, halo, O-cyclobutyl, O-oxetanyl, O—CH₂CF₃, CH₂CF₃, O—CH₂-cyclobutyl, O-cyclopentyl, NHCH₂CF₃, N(Me)CH₂CF₃, O—CH₂-(difluorocyclopropyl), and O—CH₂-cyclopropyl;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

40. A compound according to any one of claims 33 to 39, wherein R₂ and R₅ are both H.

41. A compound according to any one of claims 33 to 40, wherein R₁ is selected from H, F, Me, MeO, Cl, OH and CN, and is preferably H or F.

42. A compound according to any one of claims 33 to 41, wherein R₄ is selected from Cl, Br, and CF₃, more preferably Cl.

43. A compound according to any one of claims 33 to 42, wherein R₁ is F, R₂ is H, R₄ is Cl and R₅ is H.

44. A compound according to any one of claims 33 to 43, wherein R₂₁ is phenyl or pentafluorophenyl, more preferably, phenyl.

45. A compound according to any one of claims 32 to 44, which is selected from the following: 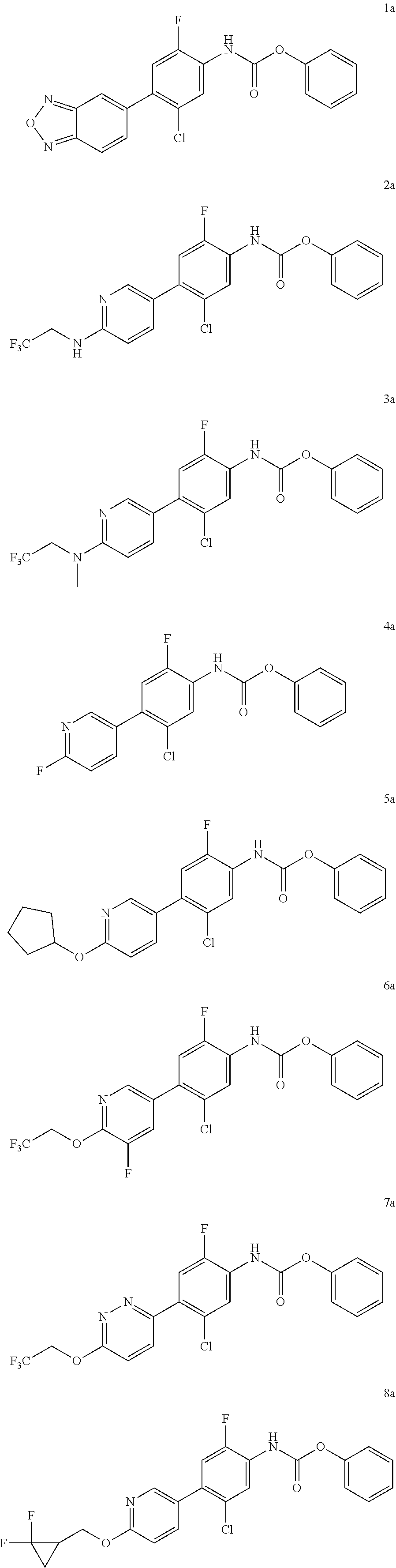 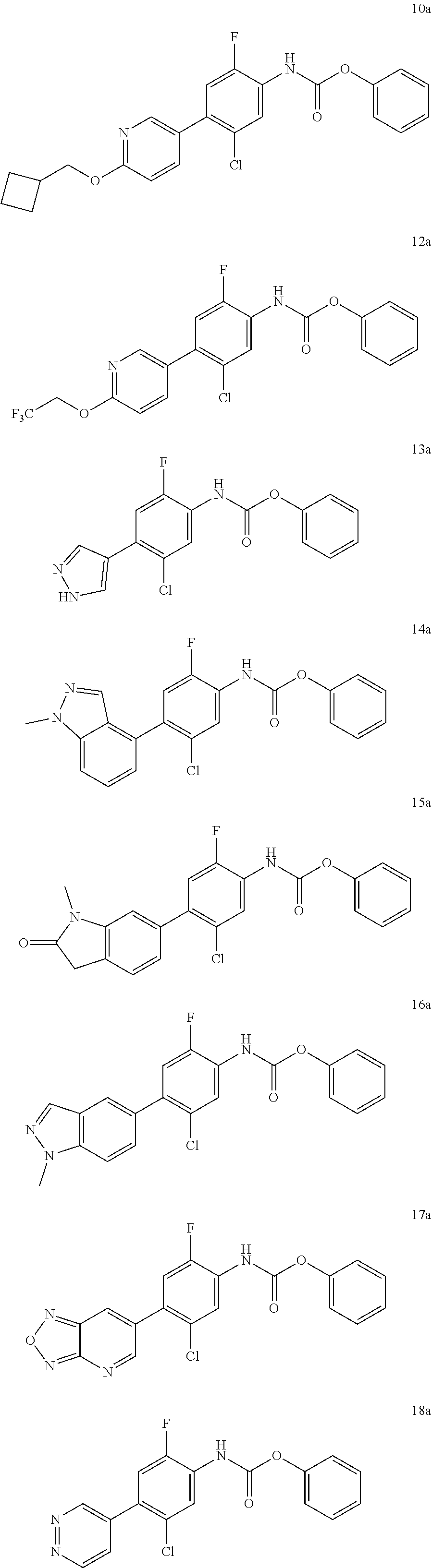 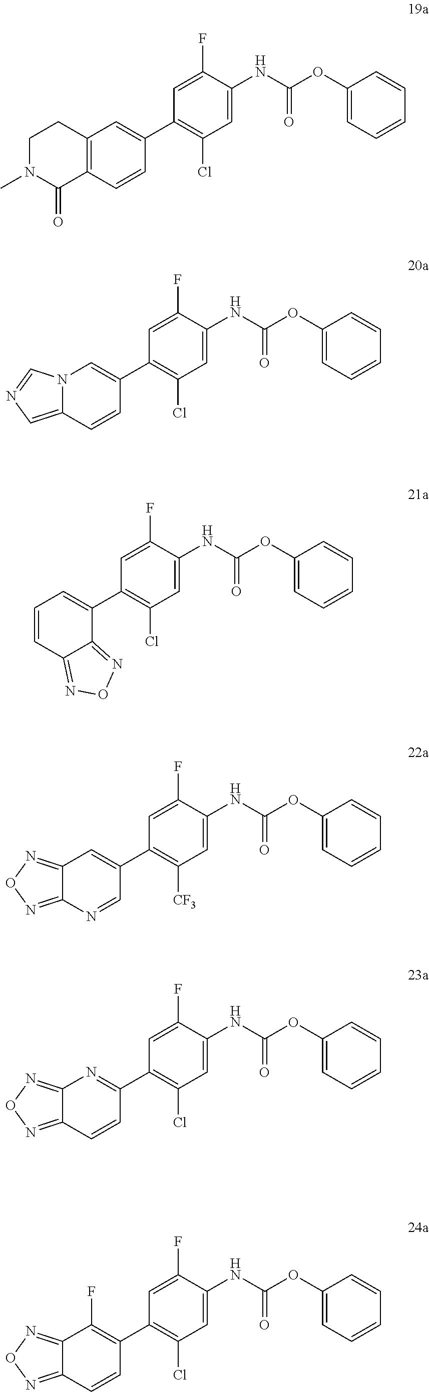 [SMILES: CN1CCc2cc(-c3cc(F)c(NC(=O)Oc4ccccc4)cc3Cl)ccc2C1=O ; O=C(Nc1cc(Cl)c(-c2ccc3cncn3c2)cc1F)Oc1ccccc1 ; O=C(Nc1cc(Cl)c(-c2cccc3nonc23)cc1F)Oc1ccccc1 ; O=C(Nc1cc(C(F)(F)F)c(-c2cnc3nonc3c2)cc1F)Oc1ccccc1 ; O=C(Nc1cc(Cl)c(-c2ccc3nonc3n2)cc1F)Oc1ccccc1 ; O=C(Nc1cc(Cl)c(-c2ccc3nonc3c2F)cc1F)Oc1ccccc1]
and enantiomers thereof, and mixtures of enantiomers thereof, including racemic mixtures, and pharmaceutically acceptable salts and solvates thereof.

46. Use of a compound according to any one of claims 32 to 45 in the preparation of a compound of formula (I) as defined in claim 1.

47. A compound selected from the following: 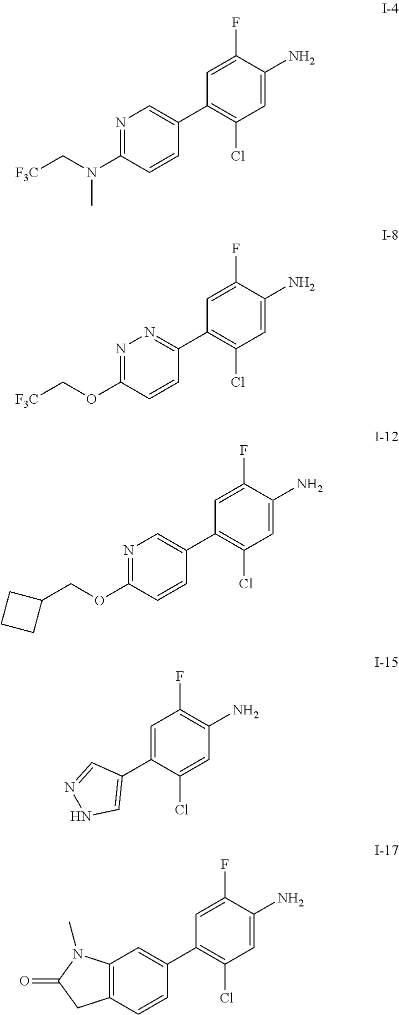 [SMILES: CN(CC(F)(F)F)c1ccc(-c2cc(F)c(N)cc2Cl)cn1 ; Nc1cc(Cl)c(-c2ccc(OCC(F)(F)F)nn2)cc1F ; Nc1cc(Cl)c(-c2ccc(OCC3CCC3)nc2)cc1F ; Nc1cc(Cl)c(-c2cn[nH]c2)cc1F ; CN1C(=O)Cc2ccc(-c3cc(F)c(N)cc3Cl)cc21]
and pharmaceutically acceptable salts and solvates thereof.

48. Use of a compound according to claim 47 in the preparation of a compound of formula (I) as defined in claim 1.
